(19) United States
(12) Patent Application Publication
(10) Pub. No.: US 2009/0042949 A1
(43) Pub. Date: Feb. 12, 2009

(54) Indoles Useful in the Treatment of Inflammation

(76) Inventors: Benjamin Pelcman, Solna (SE); Kristofer Olofsson, Solna (SE); Martins Katkevics, Riga (LV); Vita Ozola, Riga (LV); Edgars Suna, Riga (LV); Ivars Kalvins, Riga (LV); Wesley Schaal, Solna (SE)

Correspondence Address: MORGAN LEWIS & BOCKIUS LLP, 1111 PENNSYLVANIA AVENUE NW, WASHINGTON, DC, 20004, US

(21) Appl. No.: 11/795,624

(22) PCT Filed: Dec. 22, 2005
(86) PCT No.: PCT/GB05/04980

§ 371 Date: Feb. 27, 2008

Related U.S. Application Data
(60) Provisional application No. 60/644,519, filed on Jan. 19, 2005.

(51) Int. Cl. A61K 31/4439; C07D 209/42; C07D 401/14; A61P 29/00; A61K 31/404

(52) U.S. Cl. 514/339; 548/492; 514/419; 546/268.4

(57) ABSTRACT

There is provided compounds of formula I,

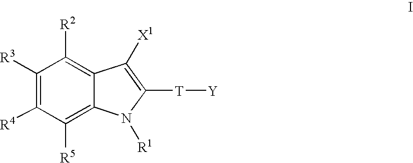

wherein X¹, T, Y, R¹, R², R³, R⁴ and R⁵ have meanings given in the description, and pharmaceutically-acceptable salts thereof, which compounds are useful in the treatment of diseases in which inhibition of the activity of a member of the MAPEG family is desired and/or required, and particularly in the treatment of inflammation.

### FIELD OF THE INVENTION

This invention relates to novel pharmaceutically-useful compounds, which compounds are useful as inhibitors of enzymes belonging to the membrane-associated proteins in the eicosanoid and glutathione metabolism (MAPEG) family. Members of the MAPEG family include the microsomal prostaglandin E synthase-1 (mPGES-1), 5-lipoxygenase-activating protein (FLAP), leukotriene C₄ synthase and microsomal glutathione S-transferases (MGST1, MGST2 and MGST3). The compounds are of potential utility in the treatment of inflammatory diseases including respiratory diseases. The invention also relates to the use of such compounds as medicaments, to pharmaceutical compositions containing them, and to synthetic routes for their production.

1. Background of the Invention

There are many diseases/disorders that are inflammatory in their nature. One of the major problems associated with existing treatments of inflammatory conditions is a lack of efficacy and/or the prevalence of side effects (real or perceived).

Inflammatory diseases that affect the population include asthma, inflammatory bowel disease, rheumatoid arthritis, osteoarthritis, rhinitis, conjunctivitis and dermatitis.

Inflammation is also a common cause of pain. Inflammatory pain may arise for numerous reasons, such as infection, surgery or other trauma. Moreover, several diseases including malignancies and cardioavascular diseases are known to have inflammatory components adding to the symptomatology of the patients.

Asthma is a disease of the airways that contains elements of both inflammation and bronchoconstriction. Treatment regimens for asthma are based on the severity of the condition. Mild cases are either untreated or are only treated with inhaled β-agonists which affect the bronchoconstriction element, whereas patients with more severe asthma typically are treated regularly with inhaled corticosteroids which to a large extent are anti-inflammatory in their nature.

Another common disease of the airways with inflammatory and bronchoconstrictive components is chronic obstructive pulmonary disease (COPD). The disease is potentially lethal, and the morbidity and mortality from the condition is considerable. At present, there is no known pharmacological treatment capable of changing the course of the disease.

The cyclooxygenase (COX) enzyme exists in two forms, one that is constitutively expressed in many cells and tissues (COX-1), and one that is induced by pro-inflammatory stimuli, such as cytokines, during an inflammatory response (COX-2).

COXs metabolise arachidonic acid to the unstable intermediate prostaglandin H₂ (PGH₂). PGH₂ is further metabolized to other prostaglandins including PGE₂, PGF_{2α}, PGD₂, prostacyclin and thromboxane A₂. These arachidonic acid metabolites are known to have pronounced physiological and pathophysiological activity including pro-inflammatory effects.

PGE₂ in particular is known to be a strong pro-inflammatory mediator, and is also known to induce fever and pain. Consequently, numerous drugs have been developed with a view to inhibiting the formation of PGEB₂, including “NSAIDs” (non-steroidal antiinflammatory drugs) and “coxibs” (selective COX-2 inhibitors). These drugs act predominantly by inhibition of COX-1 and/or COX-2, thereby reducing the formation of PGE₂.

However, the inhibition of COXs has the disadvantage that it results in the reduction of the formation of all metabolites of arachidonic acid, some of which are known to have beneficial properties. In view of this, drugs which act by inhibition of COXs are therefore known/suspected to cause adverse biological effects. For example, the non-selective inhibition of COXs by NSAIDs may give rise to gastrointestinal side-effects and affect platelet and renal function. Even the selective inhibition of COX-2 by coxibs, whilst reducing such gastrointestinal side-effects, is believed to give rise to cardiovascular problems.

An alternative treatment of inflammatory diseases that does not give rise to the above-mentioned side effects would thus be of real benefit in the clinic. In particular, a drug that inhibits (preferably selectively) the transformation of PGH₂ to the pro-inflammatory mediator PGE₂ might be expected to reduce the inflammatory response in the absence of a corresponding reduction of the formation of other, beneficial arachidonic acid metabolites. Such inhibition would accordingly be expected to alleviate the undesirable side-effects mentioned above.

PGH₂ may be transformed to PGE₂ by prostaglandin E synthases (PGES). Two microsomal prostaglandin E synthases (mPGES-1 and mPGES-2), and one cytosolic prostaglandin E synthase (cPGES) have been described.

The leukotrienes (LTs) are formed from arachidonic acid by a set of enzymes distinct from those in the COX/PGES pathway. Leukotriene B4 is known to be a strong proinflammatory mediator, while the cysteinyl-containing leukotrienes C₄, D₄ and E₄ (CysLTs) are mainly very potent bronchoconstrictors and have thus been implicated in the pathobiology of asthma. The biological activities of the CysLTs are mediated through two receptors designated CysLT₁ and CysLT₂. As an alternative to steroids, leukotriene receptor antagonists (LTRas) have been developed in the treatment of asthma. These drugs may be given orally, but do not control inflammation satisfactorily. The presently used LTRas are highly selective for CysLT₁. It may be hypothesised that better control of asthma, and possibly also COPD, may be attained if the activity of both of the CysLT receptors could be reduced. This may be achieved by developing unselective LTRas, but also by inhibiting the activity of proteins, e.g. enzymes, involved in the synthesis of the CysLTs. Among these proteins, 5-lipoxygenase, 5-lipoxygenase-activating protein (FLAP), and leukotriene C₄ synthase may be mentioned. A FLAP inhibitor would also decrease the formation of the proinflammatory LTB₄.

mPGES-1, FLAP and leukotriene C₄ synthase belong to the membrane-associated proteins in the eicosanoid and glutathione metabolism (MAPEG) family. Other members of this family include the microsomal glutathione S-transferases (MGST1, MGST2 and MGST3). For a review, c.f. P.-J. Jacobsson et al in Am. J. Respir. Crit. Care Med. 161, S20 (2000). It is well known that compounds prepared as antagonists to one of the MAPEGs may also exhibit inhibitory activity towards other family members, c.f. J. H Hutchinson et al in J. Med. Chem. 38, 4538 (1995) and D. Claveau et al in J. Immunol. 170, 4738 (2003). The former paper also describes that such compounds may also display notable cross-reactivity with proteins in the arachidonic acid cascade that do not belong to the MAPEG family, e.g. 5-lipoxygenase.

Thus, agents that are capable of inhibiting the action of mPGES-1, and thus reducing the formation of the specific arachidonic acid metabolite PGE₂, are likely to be of benefit in the treatment of inflammation. Further, agents that are capable of inhibiting the action of the proteins involved in the synthesis of the leukotrienes are also likely to be of benefit in the treatment of asthma and COPD.

2. Prior Art

Indole-based compounds have been disclosed in international patent applications WO 96/03377, WO 01/00197, WO 03/044014 and WO 03/057670, U.S. Pat. Nos. 5,189,054, 5,294,722 and 4,960,786 and European patent applications EP 429 257, EP 483 881, EP 547 556, EP 639 573 and EP 1 314 733. In particular European patent application EP 488 532 and U.S. Pat. Nos. 5,236,916 and 5,374,615 disclose 1(N)-phenylindole-2-carboxylates as antihypertensive agents and as chemical intermediates. In particular, indole-2-carboxylic amides have been disclosed as fungicides in international patent application WO 93/25524. However, none of these documents disclose or suggest the use of such compounds in the treatment of inflammation.

Indoles have also been disclosed for potential use in the treatment of inflammation in international patent applications WO 99/43672, WO 98/08818, WO 99/43654, WO 99/43651, WO 99/05104 and WO 03/029212, European patent application EP 986 666 and U.S. Pat. Nos. 6,500,853 and 6,630,496. However, there is no specific disclosure in any of these documents of indole-2-carboxylates, or derivatives thereof, in which an aromatic group is directly attached via the indole nitrogen.

International patent application WO 01/30343, and European patent application EP 186 367, also mention indoles for potential use as PPAR-E binding agents, and in the treatment of inflammation, respectively. However, these documents do not mention or suggest compounds in which the benzenoid moiety of the indole is substituted with an aromatic ring.

Various 1(N)-benzylindole-2-carboxylates and derivatives thereof are known from international patent applications WO 99/33800 as Factor Xa inhibitors; WO 99/07678, WO 99/07351, WO 00/46198, WO 00/46197, WO 00/46195 and WO 00/46199 as inhibitors of MCP-1; international patent application WO 96/18393 as inhibitors of IL-8; international patent applications WO 93/25546 and WO 94/13662, European patent application EP 535 924 A1 and U.S. Pat. No. 5,081,138 as inhibitors of leukotriene biosynthesis; international patent application WO 02/30895 as PPAR-8 binding agents; and European patent application EP 166 591 as prostaglandin antagonists. Further, international patent application WO 2005/005415 discloses such compounds for use as inhibitors of mPGES and this in the treatment of inflammation. However, there is no specific disclosure in any of these documents of indole-2-carboxylates in which an aromatic group is directly attached via the indole nitrogen.

Further, unpublished international patent applications PCT/GB2005/002404, PCT/GB2005/002391 and PCT/GB2005/002396 disclose indoles for use as inhibitors of mPGES and thus in the treatment of inflammation. However, there is no suggestion of indoles that are substituted at the 2-indolic position with a carboxylic acid amide or a derivative thereof.

Finally, international patent application WO 94/14434 discloses structurally similar indoles as endothelin receptor antagonists. There is no specific disclosure in this document of compounds with indole-2-carboxylates in which an aromatic group is directly attached via the indole nitrogen, nor of compounds in which aromatic and heteroaromatic moieties are attached to the benzenoid part of the indole via a linking group.

### DISCLOSURE OF THE INVENTION

According to the invention there is provided a compound of formula I,

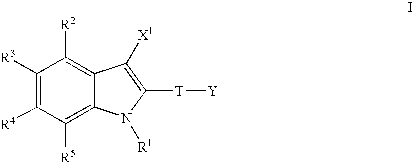

wherein
one of the groups R², R³, R⁴ and R⁵ represents -D-E and:
a) the other groups are independently selected from hydrogen, G¹, an aryl group, a heteroaryl group (which latter two groups are optionally substituted by one or more substituents selected from A), C₁₋₈ alkyl and a heterocycloalkyl group (which latter two groups are optionally substituted by one or more substituents selected from G¹ and/or Z¹); and/or
b) any two other groups which are adjacent to each other are optionally linked to form, along with two atoms of the essential benzene ring in the compound of formula I, a 3- to 8-membered ring, optionally containing 1 to 3 heteroatoms, which ring is itself optionally substituted by one or more substituents selected from halo, —R⁶, —OR⁶ and ═O;
D represents a single bond, —O—, —C(R⁷)(R⁸)—, C₂₋₄ alkylene, —C(O)— or —S(O)ₘ—;
R¹ and E independently represent an aryl group or a heteroaryl group, both of which groups are optionally substituted by one or more substituents selected from A;
R⁷ and R⁸ independently represent H, halo or C₁₋₆ alkyl, which latter group is optionally substituted by halo, or R⁷ and R⁸ may together form, along with the carbon atom to which they are attached, a 3- to 6-membered ring, which ring optionally contains a heteroatom and is optionally substituted by one or more substituents selected from halo and C₁₋₃ alkyl, which latter group is optionally substituted by one or more halo substituents;
X¹ represents H, halo, —N(R^{9k})-J-R^{10k}, —C(O)OR^{9a}, —C(O)N(R^{10b})R^{9b}, —S(O)₂N(R^{10b})R^{9b}, —C(O)N(H)C(═NR^{9c})N(R^{1d})R^{9d}, —C(O)N(H)CN, —S(O)₃R^{9c}, —P(O)(OR^{9f})₂, —P(O)(OR^{9g})N(R^{10h})R^{9h}, —P(O)(N(R¹⁰ⁱ)R⁹ⁱ)₂, —B(OR^{9j})₂, —C(O)N(H)S(O)₂R¹¹ or -Q-X²;
J represents a single bond, —C(O)— or —S(O)ₘ—;
Q represents a single bond, —O—, —C(O)—, —S(O)ₘ— or a C₁₋₈ alkylene or C₂₋₈ heteroalkylene chain, both of which latter two groups optionally contain one or more unsaturations (for example double or triple bonds) and are optionally substituted by one or more substituents selected from G¹, Z¹ and/or X³;
X² represents:
(a) an aryl group or a heteroaryl group, both of which are optionally substituted by one or more substituents selected from A and/or X³; or
(b) C₁₋₈ alkyl, C₂₋₈ heteroalkyl or a heterocycloalkyl group, all of which are optionally substituted by one or more substituents selected from G¹, Z¹ and/or X³;
X³ represents —C(O)OR^{9a}, —C(O)N(R^{10b})R^{9b}, —C(O)N(H)C(═NR^{9c})N(R^{10d})R^{9d}, —C(O)N(H)CN, —S(O)₃R^{9c}, —P(O)(OR^{9f})₂, —P(O)(OR^{9g})N(R^{10h})R^{9h}, —P(O)(N(R¹⁰ⁱ)R⁹ⁱ)₂, —B(OR^{9j})₂ or —C(O)N(H)S(O)₂R¹¹;
T represents:
(a) a single bond;
(b) a C₁₋₈ alkylene or a C₂₋₈ heteroalkylene chain, both of which latter two groups:
(i) optionally contain one or more unsaturations (for example double or triple bonds);
(ii) are optionally substituted by one or more substituents selected from G¹ and/or Z¹; and/or
(iii) may comprise an additional 3- to 8-membered ring formed between any one or more (e.g. one or two) members of the C₁₋₈ alkylene or C₂₋₈ heteroalkylene chain, which ring optionally contains 1 to 3 heteroatoms and/or 1 to 3 unsaturations (for example double or triple bonds) and which ring is itself optionally substituted by one or more substituents selected from G¹ and/or Z¹;
   (c) an arylene group or a heteroarylene group, both of which groups are optionally substituted by one or more substituents selected from A; or

### (d) -T¹-W¹-T²-;

one of T¹ and T² represents a C₁₋₈ alkylene or a C₂₋₈ heteroalkylene chain, both of which latter two groups:
(i) optionally contain one or more unsaturations (for example double or triple bonds);
(ii) are optionally substituted by one or more substituents selected from G¹ and/or Z¹; and/or
(iii) may comprise an additional 3- to 8-membered ring formed between any one or more (e.g. one or two) members of the C₁₋₈ alkylene or C₂₋₈ heteroalkylene chain, which ring optionally contains 1 to 3 heteroatoms and/or 1 to 3 unsaturations (for example double or triple bonds) and which ring is itself optionally substituted by one or more substituents selected from G¹ and/or Z¹;
   and the other represents an arylene group or a heteroarylene group, both of which groups are optionally substituted by one or more substituents selected from A;
   W¹ represents —O— or —S(O)ₘ—;
   m represents, on each occasion when mentioned above, 0, 1 or 2;
   Y represents —C(O)N(R^{10b})R^{9b}, —C(O)N(H)C(═NR^{9c})N(R^{10d})R^{9d}, —C(O)N(H)CN or —C(O)N(H)S(O)₂R¹¹;

R⁶, R^{9a} to R^{9k}, R^{10b}, R^{10d}, R^{10h}, R¹⁰ⁱ and R^{10k} independently represent, on each occasion when mentioned above:

I) hydrogen;
II) an aryl group or a heteroaryl group, both of which are optionally substituted by one or more substituents selected from B; or
III) C₁₋₈ alkyl or a heterocycloalkyl group, both of which are optionally substituted by one or more substituents selected from G¹ and/or Z¹; or
any pair of R^{9a} to R^{9k}, and R^{10b}, R^{10d}, R^{10h}, R¹⁰ⁱ or R^{10k}, may be linked together to form, along with the atom(s) and/or group(s) to which they are attached, a 3- to 8-membered ring, optionally containing 1 to 3 heteroatoms and/or 1 to 3 double bonds, which ring is optionally substituted by one or more substituents selected from G¹ and/or Z¹;
R¹¹ represents, on each occasion when mentioned above:
I) an aryl group or a heteroaryl group, both of which are optionally substituted by one or more substituents selected from B; or
II) C₁₋₈ alkyl or a heterocycloalkyl group, both of which are optionally substituted by one or more substituents selected from G¹ and/or Z¹;

A represents, on each occasion when mentioned above:

I) an aryl group or a heteroaryl group, both of which are optionally substituted by one or more substituents selected from B;
II) C₁₋₈ alkyl or a heterocycloalkyl group, both of which are optionally substituted by one or more substituents selected from G¹ and/or Z¹; or
III) a G¹ group;
G¹ represents, on each occasion when mentioned above, halo, cyano, —N₃, —NO₂, —ONO₂ or -A¹-R^{2a};
wherein A¹ represents a single bond or a spacer group selected from —C(O)A²-, —S(O)₂A³-, —N(R^{13a})A⁴ or —OA⁵-, in which:
A² represents a single bond, —O—, —N(R^{13b})— or —C(O)—;
A³ represents a single bond, —O— or —N(R^{13c})—;
A⁴ and A⁵ independently represent a single bond, —C(O)—, —C(O)N(R^{13d})—, —C(O)O—, —S(O)₂— or —S(O)₂N(R^{13e})—;
Z¹ represents, on each occasion when mentioned above, ═O, ═S, ═NOR^{12b}, ═NS(O)₂N(R^{13f})R^{12c}, ═NCN or ═C(H)NO₂;
B represents, on each occasion when mentioned above:
I) an aryl group or a heteroaryl group, both of which are optionally substituted by one or more substituents selected from G²;
II) C₁₋₈ alkyl or a heterocycloalkyl group, both of which are optionally substituted by one or more substituents selected from G² and/or Z²; or
III) a G² group;
G² represents, on each occasion when mentioned above, halo, cyano, —N₃, —NO₂, —ONO₂ or -A⁶-R^{14a};
wherein A⁶ represents a single bond or a spacer group selected from —C(O)A⁷-, —S(O)₂A⁸-, —N(R^{15a})A⁹- or —OA¹⁰-, in which:
A⁷ represents a single bond, —O—, —N(R^{15b})— or —C(O)—;
A⁸ represents a single bond, —O— or —N(R^{15c})—;
A⁹ and Alo independently represent a single bond, —C(O)—, —C(O)N(R^{15d})—; —C(O)O—, —S(O)₂— or —S(O)₂N(R^{15e})—;
Z² represents, on each occasion when mentioned above, ═O, ═S, ═NOR^{14b}, ═NS(O)₂NR^{5f})R^{14c}, ═NCN or ═C(H)NO₂;
R^{12a}, R^{12b}, R^{12c}, R^{13a}, R^{13b}, R^{13c}, R^{13d}, R^{14b}, R^{14c}, R^{15a}, R^{15b}, R^{15c},
R^{15d}, R^{15e} and R^{15f} are independently selected from:
   i) hydrogen;
   ii) an aryl group or a heteroaryl group, both of which are optionally substituted by one or more substituents selected from G³;
   iii) C₁₋₈ alkyl or a heterocycloalkyl group, both of which are optionally substituted by G³ and/or Z³; or
   any pair of R^{12a} to R^{12c} and R^{13a} to R^{13f}, and/or R^{14a} to R^{4c} and R^{15a} to R^{15f}, may, for example when present on the same or on adjacent atoms, be linked together to form with those, or other relevant, atoms a further 3- to 8-membered ring, optionally containing 1 to 3 heteroatoms and/or 1 to 3 double bonds, which ring is optionally substituted by one or more substituents selected from G³ and/or Z³;
   G³ represents, on each occasion when mentioned above, halo, cyano, —N₃, —NO₂, —ONO₂ or -A¹¹-R^{16a};
   wherein A¹¹ represents a single bond or a spacer group selected from —C(O)A¹², —S(O)₂A¹³-, —N(R^{17a})A¹⁴- or —OA¹⁵-, in which:
   A¹² represents a single bond, —O—, —N(R^{17b})— or —C(O)—;
   A¹³ represents a single bond, —O— or —N(R^{17c})—;
   A¹⁴ and A¹⁵ independently represent a single bond, —C(O)—, —C(O)N(R^{17d})—, —C(O)O—, —S(O)₂— or —S(O)₂N(R^{17e})—;
   Z³ represents, on each occasion when mentioned above, ═O, ═S, ═NOR^{16b}, ═NS(O)₂N(R^{17f})R^{16c}, ═NCN or ═C(H)NO₂;
   R^{16a}, R^{16b}, R^{16c}, R^{17a}, R^{17b}, R^{17c}; R^{17d}R^{17e} and R^{17f} are independently selected from:
   i) hydrogen;
   ii) C₁₋₆ alkyl or a heterocycloalkyl group, both of which groups are optionally substituted by one or more substituents selected from halo, C₁₋₄ alkyl, —N(R^{18a})R^{19a}, —OR^{18b} and ═O; and
   iii) an aryl or heteroaryl group, both of which are optionally substituted by one or more substituents selected from halo, C₁₋₄ alkyl, —N(R^{18c})R^{19b} and —OR^{18d}; or any pair of R¹⁶ to R^{6c} and R^{17a} to R^{17f} may, for example when present on the same or on adjacent atoms, be linked together to form with those, or other relevant, atoms a further 3- to 8-membered ring, optionally containing 1 to 3 heteroatoms and/or 1 to 3 double bonds, which ring is optionally substituted by one or more substituents selected from halo, C₁₋₄ alkyl, —N(R^{18e})R^{19c}, —OR^{18f} and ═O;

R^{18a}, R^{18b}, R^{18c}, R^{8d}, R^{8c}, R^{8d}, R^{8f}, R^{9a}, R^{9b} and R^{19c} are independently selected from hydrogen and C₁₋₄ alkyl, which latter group is optionally substituted by one or more halo groups;

or a pharmaceutically-acceptable salt thereof,
provided that, when R¹ represents 3,4-dimethoxyphenyl, T both represent single bonds, X¹, R², R⁴ and R⁵ all represent H, R³ represents -D-E, in which D represents a single bond and E represents phenyl, or D represents —O— and E represents 4-chlorophenyl, and Y represents —C(O)N(R^{10b})R^{9b}, then R^{9b} and R^{10b} are not linked together to form, along with the N atom to which they are attached, a 4-morpholin-1-yl ring, which compounds and salts are referred to hereinafter as “the compounds of the invention”.

Pharmaceutically-acceptable salts include acid addition salts and base addition salts. Such salts may be formed by conventional means, for example by reaction of a free acid or a free base form of a compound of formula I with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound of the invention in the form of a salt with another counter-ion, for example using a suitable ion exchange resin.

Compounds of the invention may contain double bonds and may thus exist as E (entgegen) and Z (zusammen) geometric isomers about each individual double bond. All such isomers and mixtures thereof are included within the scope of the invention.

Compounds of the invention may also exhibit tautomerism. All tautomeric forms and mixtures thereof are included within the scope of the invention.

Compounds of the invention may also contain one or more asymmetric carbon atoms and may therefore exhibit optical and/or diastereoisomerism. Diastereoisomers may be separated using conventional techniques, e.g. chromatography or fractional crystallisation. The various stereoisomers may be isolated by separation of a racemic or other mixture of the compounds using conventional, e.g. fractional crystallisation or HPLC, techniques. Alternatively the desired optical isomers may be made by reaction of the appropriate optically active starting materials under conditions which will not cause racemisation or epimerisation (i.e. a ‘chiral pool’ method), by reaction of the appropriate starting material with a ‘chiral auxiliary’ which can subsequently be removed at a suitable stage, by derivatisation (i.e. a resolution, including a dynamic resolution), for example with a homochiral acid followed by separation of the diastereomeric derivatives by conventional means such as chromatography, or by reaction with an appropriate chiral reagent or chiral catalyst all under conditions known to the skilled person. All stereoisomers and mixtures thereof are included within the scope of the invention.

Unless otherwise specified, C_{1-q} alkyl, and C_{1-q} alkylene, groups (where q is the upper limit of the range) defined herein may be straight-chain or, when there is a sufficient number (i.e. a minimum of two or three, as appropriate) of carbon atoms, be branched-chain, and/or cyclic (so forming, in the case of alkyl, a C_{3-q}-cycloalkyl group or, in the case of alkylene, a C_{3-q} cycloalkylene group. Further, when there is a sufficient number (i.e. a minimum of four) of carbon atoms, such groups may also be part cyclic. Such alkyl and alkylene groups may also be saturated or, when there is a sufficient number (i.e. a minimum of two) of carbon atoms, be unsaturated (forming, for example, in the case of alkyl, a C_{2-q} alkenyl or a C_{2-q} alkynyl group or, in the case of alkylene, a C_{2-q} alkenylene or a C_{2-q} alkynylene group).

C_{3-q} cycloalkyl groups (where q is the upper limit of the range) that may be mentioned may be monocyclic or bicyclic alkyl groups, which cycloalkyl groups may further be bridged (so forming, for example, fused ring systems such as three fused cycloalkyl groups). Such cycloalkyl groups may be saturated or unsaturated containing one or more double or triple bonds (forming for example a C_{3-q} cycloalkenyl or a C8-q cycloalkynyl group). Substituents may be attached at any point on the cycloalkyl group. Further in the case where the substituent is another cyclic compound, then the cyclic substituent may be attached through a single atom on the cycloalkyl group, forming a so-called “spiro”-compound.

C₂₋₈ heteroalkyl groups and C₂₋₈ heteroalkylene chains include C₂₋₈ alkyl groups, and C₂₋₈ alkylene chains, respectively, that are interrupted by one or more heteroatom groups selected from —O—, —S— or —N(R²⁰)—, in which R²⁰ represents C₁₋₄ alkyl, optionally substituted by one or more halo (e.g. fluoro) groups.

The term “halo”, when used herein, includes fluoro, chloro, bromo and iodo.

Heterocycloalkyl groups that may be mentioned include non-aromatic monocyclic and bicyclic groups (which groups may further be bridged) in which at least one (e.g. one to four) of the atoms in the ring system is other than carbon (i.e. a heteroatom), and in which the total number of atoms in the ring system is between three and twelve (e.g. between five and ten). Further, such heterocycloalkyl groups may be saturated or unsaturated containing one or more double and/or triple bonds, forming for example a C_{2-q} heterocycloalkenyl (where q is the upper limit of the range) or a C_{3-q} heterocycloalkynyl group. C_{2-q} heterocycloalkyl groups that may be mentioned include 7-azabicyclo[2.2.1]heptanyl, 6-azabicyclo[3.1.1]heptanyl, 6-azabicyclo[3.2.1]octanyl, 8-azabicyclo[3.2.1]octanyl, aziridinyl, azetidinyl, dihydropyranyl, dihydropyridyl, dihydropyrrolyl (including 2,5-dihydropyrrolyl), dioxolanyl (including 1,3-dioxolanyl), dioxanyl (including 1,3-dioxanyl and 1,4-dioxanyl), dithianyl (including 1,4-dithianyl), dithiolanyl (including 1,3-dithiolanyl), imidazolidinyl, imidazolinyl, morpholinyl, 7-oxabicyclo[2.2.1]heptanyl, 6-oxabicyclo[3.2.1]octanyl, oxetanyl, oxiranyl, piperazinyl, piperidinyl, pyranyl, pyrazolidinyl, pyrrolidinonyl, pyrrolidinyl, pyrrolinyl, quinuclidinyl, sulfolanyl, 3-sulfolenyl, tetrahydropyranyl, tetrahydrofuranyl, tetrahydropyridyl (such as 1,2,3,4-tetrahydropyridyl and 1,2,3,6-tetrahydropyridyl), thietanyl, thiiranyl, thiolanyl, thiomorpholinyl, trithianyl (including 1,3,5-trithianyl), tropanyl and the like. Substituents on heterocycloalkyl groups may, where appropriate, be located on any atom in the ring system including a heteroatom. Further, in the case where the other substituent is another cyclic compound, then the cyclic compound may be attached through a single atom on the heterocycloalkyl group, forming a so-called “spiro”-compound. The point of attachment of heterocycloalkyl groups may be via any atom in the ring system including (where appropriate) a heteroatom (such as a nitrogen atom), or an atom on any fused carbocyclic ring that may be present as part of the ring system. Heterocycloalkyl groups may also be in the N- or S-oxidised form.

For the avoidance of doubt, the term “bicyclic”, when employed in the context of cycloalkyl and heterocycloalkyl groups refers to such groups in which the second ring is formed between two adjacent atoms of the first ring. The term “bridged”, when employed in the context of cycloalkyl or heterocycloalkyl groups refers to monocyclic or bicyclic groups in which two non-adjacent atoms are linked by either an alkylene or heteroalkylene chain (as appropriate).

Aryl groups that may be mentioned include C₆₋₁₄ (such as C₆₋₁₃ (e.g. C₆₋₁₀)) aryl groups. Such groups may be monocyclic, bicyclic or tricyclic and have between 6 and 14 ring carbon atoms, in which at least one ring is aromatic. C₆₋₁₄ aryl groups include phenyl, naphthyl and the like, such as 1,2,3,4-tetrahydronaphthyl, indanyl, indenyl and fluorenyl. The point of attachment of aryl groups may be via any atom of the ring system. However, when aryl groups are bicyclic or tricyclic, they are linked to the rest of the molecule via an aromatic ring.

Heteroaryl groups that may be mentioned include those which have between 5 and 14 (e.g. 10) members. Such groups may be monocyclic, bicyclic or tricyclic,

provided that at least one of the rings is aromatic and wherein at least one (e.g. one to four) of the atoms in the ring system is other than carbon (i.e. a heteroatom). Heterocyclic groups that may be mentioned include benzothiadiazolyl (including 2,1,3-benzothiadiazolyl), isothiochromanyl and, more preferably, acridinyl, benzimidazolyl, benzodioxanyl, benzodioxepinyl, benzodioxolyl (including 1,3-benzodioxolyl), benzofuranyl, benzofurazanyl, benzothiazolyl, benzoxadiazolyl (including 2,1,3-benzoxadiazolyl), benzoxazinyl (including 3,4-dihydro-2H-1,4-benzoxazinyl), benzoxazolyl, benzomorpholinyl, benzoselenadiazolyl (including 2,1,3-benzoselenadiazolyl), benzothienyl, carbazolyl, chromanyl, cinnolinyl, furanyl, imidazolyl, imidazo[1,2-a]pyridyl, indazolyl, indolinyl, indolyl, isobenzofuranyl, isochromanyl, isoindolinyl, isoindolyl, isoquinolinyl, isothiaziolyl, isoxazolyl, naphthyridinyl (including 1,6-naphthyridinyl or, preferably, 1,5-naphthyridinyl and 1,8-naphthyridinyl), oxadiazolyl (including 1,2,3-oxadiazolyl, 1,2,4-oxadiazolyl and 1,3,4-oxadiazolyl), oxazolyl, phenazinyl, phenothiazinyl, phthalazinyl, pteridinyl, purinyl, pyrazinyl, pyrazolyl, pyridazinyl, pyridyl, pyrimidinyl, pyrrolyl, quinazolinyl, quinolinyl, quinolizinyl, quinoxalinyl, tetrahydroisoquinolinyl (including 1,2,3,4-tetrahydroisoquinolinyl and 5,6,7,8-tetrahydroisoquinolinyl), tetrahydroquinolinyl (including 1,2,3,4-tetrahydroquinolinyl and 5,6,7,8-tetrahydroquinolinyl), tetrazolyl, thiadiazolyl (including 1,2,3-thiadiazolyl, 1,2,4-thiadiazolyl and 1,3,4-thiadiazolyl), thiazolyl, thiochromanyl, thienyl, triazolyl (including 1,2,3-triazolyl, 1,2,4-triazolyl and 1,3,4-triazolyl) and the like. Substituents on heteroaryl groups may, where appropriate, be located on any atom in the ring system including a heteroatom. The point of attachment of heteroaryl groups may be via any atom in the ring system including (where appropriate) a heteroatom (such as a nitrogen atom), or an atom on any fused carbocyclic ring that may be present as part of the ring system. Heteroaryl groups may also be in the N— or S— oxidised form.

Heteroatoms that may be mentioned include phosphorus, silicon, boron, tellurium, selenium and, preferably, oxygen, nitrogen and sulphur.

For the avoidance of doubt, “heterocycloalkylene”, “arylene”, “heteroarylene” and “cycloalkylene” groups as defined herein comprise “linking” groups in which a heterocycloalkyl, an aryl, a heteroaryl, or a cycloalkyl, group (each of which are as defined hereinbefore), serves the purpose of linking two different parts of a compound of the invention together, in exactly the same way as an alkylene group can be said to constitute a “linking” (i.e. a divalent) alkyl group. Thus, for example, a phenyl group that serves the purpose of linking two substituents within, or parts of, a compound of the invention together would be classified in the context of the present invention as a “phenylene” group.

For the avoidance of doubt, in cases in which the identity of two or more substituents in a compound of the invention may be the same, the actual identities of the respective substituents are not in any way interdependent. For example, in the situation in which R¹ and E are both aryl groups substituted by one or more C₁₋₈ alkyl groups, the alkyl groups in question may be the same or different.

Similarly, when groups are substituted by more than one substituent as defined herein, the identities of those individual substituents are not to be regarded as being interdependent. For example, when E and/or R¹ represents e.g. an aryl group substituted by G¹ in addition to, for example, C₁₋₈ alkyl, which latter group is substituted by G¹, the identities of the two G¹ groups are not to be regarded as being interdependent.

For the avoidance of doubt, when a term such as “R^{9a} to R^{9k}” is employed herein, this will be understood by the skilled person to mean R^{9a}, R^{9b}, R^{9c}, R^{9d}, R^{9e}, R^{9f}, R^{9g}, R^{9h}, R⁹ⁱ, R^{9j} and R^{9k} inclusively.

Any pair of R^{9a} to R^{9k}, and R^{10b}, R^{10d}, R^{10h}, R¹⁰ⁱ and R^{10k}, may be linked together to form a ring as hereinbefore defined. Thus R^{9a} to R^{9k}, R^{10b}, R^{10d}, R^{10h}, R¹⁰ⁱ and R^{10k} groups may be attached to (a) a single nitrogen atom (e.g. R^{9b} and R^{9b}), or (b) a nitrogen atom and a J group (i.e. R^{9k} and R^{10k}), which also form part of the ring, or two R^{9a} to R^{9k} groups may be attached to different oxygen atoms (for example in a 1,3-relationship) all of which may form part of the ring.

When X¹ represents -Q-X², Q represents C₁₋₈ alkylene or C₂₋₈ heteroalkylene and X² represents C₁₋₈ alkyl or C₂₋₈ heteroalkyl, it is preferred that the total number of carbon atoms in the group -Q-X² does not exceed 12, such as 10 (e.g. 8).

Compounds of the invention that may be mentioned include those in which X¹ represents H, halo, 1 N(R^{9k})-J-R^{10k}, —C(O)OR^{9a}, —C(O)N(R^{10b})R^{9b}, —C(O)N(H)C(═NR^{9c})N(R^{10d})R^{9d}, —C(O)N(H)CN, —S(O)₃R^{9e}, —P(O)(OR^{9f})₂, —P(O)(OR^{9g})N(R^{h})R^{9h}, —P(O)(N(R¹⁰ⁱ)R⁹ⁱ)₂, —B(OR^{9j})₂, —C(O)N(H)S(O)₂R¹¹ or -Q-X²

Further compounds of the invention that may be mentioned include those in which when X¹ represents —N(R^{9k})-J-R^{10k}, then R^{9k} and R^{10k} are not linked together with the nitrogen atom to which they are attached to form a morpholinyl (e.g. a 4-morpholin-1-yl) group.

Further compounds of the invention that may be mentioned include those in which, when Y represents —C(O)N(R^{10b})R^{9b}, then it is not:

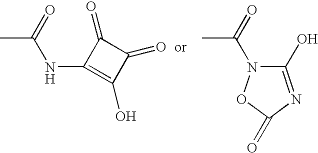

Preferred compounds of the invention include those in which:

T represents a single bond or linear or branched C₁₋₃ alkylene, which latter group is optionally substituted by one or more Z¹ substituent;
Y represents —C(O)N(R^{10b})R^{9b}, —C(O)N(H)C(═NR^{9c})N(R^{10d})R^{9d} or —C(O)N(H)S(O)₂R¹¹;
R^{9a} to R^{9k} independently represent H or C₁₋₆ (e.g. C₁₋₃) alkyl;
R^{10b}, R^{10d}, R^{10h}, R¹⁰ⁱ and R^{10k} independently represent H or C₁₋₆ (e.g. C₁₋₄) alkyl optionally substituted by one or more G¹ groups;
or any pair of R^{9a} to R^{9k}, and R^{10b}, R^{10d}, R^{10k}, R^{10h}, R¹⁰ⁱ or R^{10k} are linked to form a 4- to 7-membered (e.g. 5- or 6-membered) ring, which ring may, for example preferably, contain (in addition to the nitrogen atom to which any one of R^{9a} to R^{9k} is attached) a further heteroatom (e.g. nitrogen or oxygen) and which ring is optionally substituted by one or more Z¹ groups;
R¹¹ represents C₁₋₃ alkyl;
X¹ represents H, —C(O)OR^{9a}, —P(O)(OR^{9f})₂ or -Q-X²;
Q represents a single bond, C₁₋₈ alkylene or nitrogen-containing C₂₋₆ heteroalkylene, which latter two groups are optionally substituted with one or more X³ and/or G¹ groups;
X² represents an aryl group, a heteroaryl group, C₁₋₆ (e.g. C₁₋₄) alkyl or a heterocycloalkyl group all of which are optionally substituted with one or more G¹, Z¹ and/or, preferably, X³ groups;
X³ represents —C(O)OR^{9a} or —P(O)(OR^{9f})₂;
A represents G¹ or C₁₋₇ alkyl optionally substituted by one or more G¹ groups;
G¹ represents cyano, —NO₂ or, more preferably, halo or -A¹-R^{12a};
A¹ represents a single bond or, preferably, a spacer group as hereinbefore defined;
A⁴ and A⁵ independently represent —C(O)—, —C(O)N(R^{13d})— or, preferably, —C(O)O— or a single bond;
R^{12a} to R^{12c} independently represent a heterocycloalkyl group (such as C₄₋₈ heterocycloalkyl, which group contains one nitrogen atom and, optionally, a further nitrogen or oxygen atom), a heteroaryl group (which latter two groups are optionally substituted by one or more groups selected from G³ and/or, in the case of heterocycloalkyl, Z³) or, more preferably, H or C₁₋₆ alkyl optionally substituted by one or more G³ and/or Z³ groups;
R^{13a} to R^{13f} independently represent H or C₁₋₆ alkyl optionally substituted by one or more G³ and/or Z³ groups;
Z¹ represents ═NOR^{12b}, ═NCN or, preferably, ═O;
G² represents cyano, —N₃ or, more preferably, halo, —NO₂ or -A⁶-R^{14a};
A⁶ represents —N(R^{15a})A⁹- or —OA¹⁰-;
A⁹ represents —C(O)N(R^{15d})—, —C(O)O— or, more preferably, a single bond or —C(O)—;
A¹⁰ represents a single bond;
Z² represents ═NOR^{14b} or ═NCN or, more preferably, ═O;
G³ represents halo, —NO₂ or -A¹¹-R^{16a};
A¹¹ represents —N(R^{17a})—, —O— or, more preferably, a single bond;
R^{16a} to R^{16c} independently represent an optionally substituted aryl group;
Z³ represents ═O;
J represents a single bond, —C(O)— or —S(O)₂;
when any of R^{16a}, R^{16b}, R^{16c}, R^{17a}, R^{17b}, R^{17c}, R^{17d}, R^{17e} and R^{17f} represents optionally substituted C₁₋₆ alkyl, the optional substituent is one or more halo groups;
when any one of R^{18a}, R^{18b}, R^{18c}, R^{18d}, R^{18e}, R^{18f}, R^{19a}, R^{9b} and R^{19c} represents optionally substituted C₁₋₄ alkyl, the optional substituent is one or more fluoro groups.

Preferred aryl and heteroaryl groups that R¹, X² (when X² represents an aryl or heteroaryl group) and E may represent include optionally substituted phenyl, naphthyl, pyrrolyl, furanyl, thienyl, pyrazolyl, imidazolyl (e.g 1-imidazolyl, 2-imidazolyl or 4-imidazolyl), oxazolyl, isoxazolyl, thiazolyl, pyridyl (e.g. 2-pyridyl, 3-pyridyl or 4-pyridyl), indazolyl, indolyl, indolinyl, isoindolinyl, quinolinyl, 1,2,3,4-tetrahydroquinolinyl, isoquinolinyl, 1,2,3,4-tetrahydroisoquinolinyl, quinolizinyl, benzofuranyl, isobenzofuranyl, chromanyl, benzothienyl, pyridazinyl, pyrimidinyl, pyrazinyl, indazolyl, benzimidazolyl, quinazolinyl, quinoxalinyl, 1,3-benzodioxolyl, tetrazolyl, benzothiazolyl, and/or benzodioxanyl groups.

Preferred values of R¹ and E include optionally substituted pyridyl (e.g. 2-pyridyl), phenyl and imidazolyl.

Optional substituents on R¹, R², R³, R⁴, R⁵, X¹ and E groups are preferably selected from:

halo (e.g. fluoro, chloro or bromo);
cyano;

### —NO₂;

C₁₋₆ alkyl, which alkyl group may be linear or branched (e.g. C₁₋₄ alkyl (including ethyl, n-propyl, isopropyl, n-butyl or, preferably, methyl or t-butyl), n-pentyl, isopentyl, n-hexyl or isohexyl), cyclic (e.g. cyclopropyl, cyclobutyl, cyclopentyl or cyclohexyl), part-cyclic (e.g. cyclopropylmethyl), unsaturated (e.g. 1-propenyl, 2-propenyl, 1-butenyl, 2-butenyl, 3-butenyl, 1-pentenyl, 2-pentenyl, 4-pentenyl or 5-hexenyl) and/or optionally substituted with one or more halo (e.g. fluoro) group (so forming, for example, fluoromethyl, difluoromethyl or, preferably, trifluoromethyl);
heterocycloalkyl, such as a C₄₋₅ heterocycloalkyl group, preferably containing a nitrogen atom and, optionally, a further nitrogen or oxygen atom, so forming for example morpholinyl (e.g. 4-morpholinyl), piperazinyl (e.g. 4-piperazinyl) or piperidinyl (e.g. 1-piperidinyl and 4-piperidinyl) or pyrrolidinyl (e.g. 1-pyrrolidinyl), which heterocycloalkyl group is optionally substituted by one or more (e.g. one or two) substituents selected from C₁₋₃ alkyl (e.g. methyl) and ═O;

### —OR²¹; and

### —N(R²¹)R²²;

wherein R²¹ and R²² independently represent, on each occasion when mentioned above, H or C₁₋₆ alkyl, such as methyl, ethyl, n-propyl, n-butyl, t-butyl, cyclopropyl, cyclobutyl, cyclohexyl or, preferably, isopropyl or cyclopentyl (which alkyl groups are optionally substituted by one or more halo (e.g. fluoro) groups (to form e.g. a trifluoromethyl group)).

Preferred values of R^{9a} to R^{9k} include C₁₋₄ alkyl and, particularly, H. Preferred values of R^{10b}, R^{10d}, R^{10h}, R¹⁰ⁱ and R^{10k} include heteroaryl optionally substituted by B, or, preferably, C₁₋₃ alkyl and H.

More preferred compounds include those in which:

one or R⁴ and, more preferably, R³ represents -D-E and the other (more preferably) represents H;
D represents a single bond or —O—;
R² and/or R⁵ represent H;
T represents C₁₋₃ alkylene (e.g. methylene), phenylene or, more preferably, a single bond;
Y represents —C(O)N(R^{10b})R^{9b}, —C(O)N(H)C(═NH)NH₂ or —C(O)N(H)S(O)₂R¹¹;
R^{9a} to R^{9k} independently represent H or C₁₋₂ alkyl (e.g. methyl);
R^{10b}, R^{10d}, R^{10h}, R¹⁰ⁱ, and R^{10k} independently represent heteroaryl (such as isoxazolyl (e.g. 3-isoxazolyl), tetrazolyl (e.g. 5-tetrazolyl), thiadiazolyl (e.g. 1,3,4-thiadiazol-2-yl) or triazolyl (e.g. 1,2,4-triazol-4-yl)) optionally substituted by one or more (e.g. one) B groups, or, more preferably, H or C₁₋₄ (e.g. C₁₋₃) alkyl (e.g. isobutyl, cyclopropylmethyl, preferably, n-propyl, cyclopropyl, isopropyl or, more preferably, methyl or ethyl) optionally substituted by one or more G¹ groups;
R¹¹ represents C₁₋₂ alkyl (e.g. methyl);
X¹ represents —C(O)OR^{9a}, preferably, halo (e.g. chloro or fluoro), Q-X² or, more preferably, H;
X² represents C₁₋₃ alkyl (e.g. methyl) or heterocycloalkyl, both of which are optionally substituted by one or more G¹ and/or X³ groups;
A represents G¹ or C₁₋₆ alkyl (e.g. methyl or 1-butyl) optionally substituted by one or more G¹ groups;
G¹ represents fluoro, chloro or -A¹-R^{12a};
A² and A³ independently represent —O—;
A⁴ represents a single bond, preferably, —C(O)— or, more preferably, —C(O)O—;
A⁵ represents a single bond;
B represents C₁₋₃ alkyl (e.g. methyl);
R^{12a} to R^{12c} independently represent a heteroaryl group (such as imidazolyl (e.g. 4- or 2-imidazolyl), pyridyl (e.g. 3-pyridyl or 4-pyridyl) or tetrazolyl (e.g. 5-tetrazolyl) or a C₄₋₅ heterocycloalkyl group (e.g. pyrrolidinyl, piperidinyl, piperazinyl and morpholinyl) or, more preferably, H or C₁₋₅ alkyl (e.g. methyl, isopropyl or cyclopentyl), which alkyl group is optionally substituted with one or more G³ groups;
R^{13a} to R^{13f} independently represent H or C₁₋₂ alkyl (e.g. methyl);
G³ represents halo (e.g. fluoro) or -A¹¹-R^{16a};
A¹¹ represents a single bond;
R¹⁶ to R^{16c} independently represent phenyl.

Particularly preferred values of X² include C₁₋₃ alkyl (e.g. methyl), which group is unsubstituted or, preferably, substituted by one or more halo (e.g. fluoro or chloro) groups so forming, for example, a trifluoromethyl group.

Particularly preferred values of R^{10b} (e.g. when Y represents —C(O)N(R^{11b})R^{9b}) include —C(═NH)NH₂, —CH₂C(O)OH, —CHFC(O)OH, —CF₂C(O)OH, —C₂H₄C(O)OH (e.g. —CH₂CH₂COOH and —CH(CH₃)C(O)OH), —CH₂CH(N(H)C(O)OCH₂-phenyl)C(O)OH, —C₂H₄S(O)₂OH, 3-isoxazolyl (e.g. 5-methyl isoxazol-3-yl), 5-tetrazolyl, 1,3,4-thiadiazol-2-yl, triazol-4-yl, —C₂H₄NH₂, cyclopropyl-C(O)OH (e.g. —C(CH₂—CH₂)C(O)OH, i.e. a cyclopropyl group substituted by —C(O)OH α to the point of attachment of the R^{10b} group to the essential nitrogen atom of the —C(O)N(R^{10b})R^{9b} group), —C(CH₃)₂C(O)OH, —CH₂CH(CH₃)C(O)OH, —CH₂C(CH₃)₂C(O)OH and —CH₂-cyclopropyl-C(O)OH (in which the —C(O)OH group may be attached to any of the carbon atoms of the cyclopropyl group).

Particularly preferred values of R¹ include 4-cyclopentoxyphenyl, 4-isopropoxyphenyl and 4-cyc lopropoxyphenyl.

Preferred values of E include 4-tert-butylphenyl, 3-chlorophenyl, 5-trifluoromethylpyrid-2-yl, 4-trifluoromethylphenyl and 4-trifluoromethoxyphenyl.

Particularly preferred compounds of the invention include those of the examples described hereinafter.

Compounds of the invention may be made in accordance with techniques that are well known to those skilled in the art, for example as described hereinafter.

According to a further aspect of the invention there is provided a process for the preparation of a compound of formula I, which process comprises:

(i) reaction of a compound of formula II,

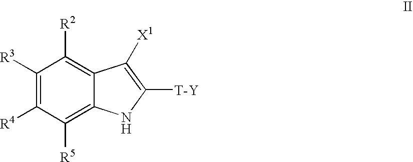

wherein X¹, R², R³, R⁴, R⁵, T and Y are as hereinbefore defined, with a compound of formula III,

R¹L¹ III

wherein L¹ represents a suitable leaving group such as chloro, bromo, iodo, a sulfonate group (e.g. —OS(O)₂CF₃, —OS(O)₂CH₃, —OS(O)₂PhMe or a nonaflate) or —B(OH)₂ and R¹ is as hereinbefore defined, for example optionally in the presence of an appropriate metal catalyst (or a salt or complex thereof) such as Cu, Cu(OAc)₂, CuI (or CuI/diamine complex), Pd(OAc)₂, Pd₂(dba)₃ or NiCl₂ and an optional additive such as Ph₃P, 2,2′-bis(diphenylphosphino)-1,1′-binaphthyl, xantphos, NaI or an appropriate crown ether such as 18-crown-6-benzene, in the presence of an appropriate base such as NaH, Et₃N, pyridine, N,N′-dimethylethylenediamine, Na₂CO₃, K₂CO₃, K₃PO₄, Cs₂CO₃, t-BuONa or t-BuOK (or a mixture thereof), in a suitable solvent (e.g. dichloromethane, dioxane, toluene, ethanol, isopropanol, dimethylformamide, ethylene glycol, ethylene glycol dimethyl ether, water, dimethylsulfoxide, acetonitrile, dimethylacetamide, N-methylpyrrolidinone, tetrahydrofuran or a mixture thereof) or in the absence of an additional solvent when the reagent may itself act as a solvent (e.g. when R¹ represents phenyl and L¹ represents bromo, i.e. bromobenzene). This reaction may be carried out at room temperature or above (e.g. at a high temperature, such as the reflux temperature of the solvent system that is employed) or using microwave irradiation;
(ii) for compounds of formula I in which X¹ represents —C(O)N(H)C(═NR^{9c})N(R^{10d})R^{9d}, —C(O)N(H)CN, —C(O)N(H)S(O)₂R¹¹ or, preferably, —C(O)OR^{9a}, —C(O)N(R^{10b})R^{9b}, —S(O)₂N(R^{10b})R^{9b}, —S(O)₃R^{9e}, —P(O)(OR^{9f})₂, —P(O)(OR^{9g})N(R^{10h})R^{9h}, —P(O)(N(R¹⁰ⁱ)R⁹ⁱ)₂, —B(OR^{9j})₂ or -Q-X², in which Q is a single bond, —C(O)—, C₁₋₈ alkylene or C₂₋₈ heteroalkylene, reaction of a compound of formula IV,

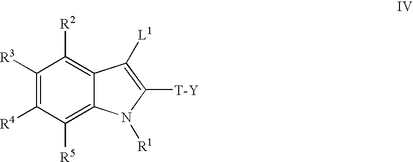

wherein L¹, R¹, R², R³, R⁴, R⁵, T and Y are as hereinbefore defined, with a compound of formula V,

X^{1a}-L² V

wherein X^{1a} represents —C(O)N(H)C(═NR^{9c})N(R^{10d})R^{9d}, —C(O)N(H)CN, —C(O)N(H)S(O)₂R¹¹ or, preferably, —C(O)OR^{9a}, —C(O)N(R^{10b})R^{9b}, —S(O)₂N(R^{10b})R^{9b}, S(O)₃R^{9e}, —P(O)(OR^{9f})₂, —P(O)(OR^{9g})N(R^{10h})R^{9h}, —P(O)(N(R¹⁰ⁱ)R⁹ⁱ)₂, —B(OR^{9j})₂ or -Q-X², in which latter case Q is a single bond, —C(O)—, C₁₋₈ alkylene or C₂₋₈ heteroalkylene, L² represents a suitable leaving group such as chloro, bromo, iodo, —B(OH)₂ or a protected derivative thereof, for example a 4,4,5,5-tetramethyl-1,3,2-dioxaborolan-2-yl group, 9-borabicyclo[3.3.1]-nonane (9-BBN), —Sn(alkyl)₃ (e.g. —SnMe₃ or —SnBu₃), or a similar group known to the skilled person, and R^{9a} to R^{9k}, R^{10b}, R^{10d}, R^{10h}, R¹⁰ⁱ, R^{10k} and R¹¹ are as hereinbefore defined. The skilled person will appreciate that L¹ and L² will be mutually compatible. In this respect, preferred leaving groups for compounds of formula V in which X^{1a} is -Q-X² and Q is —C(O)— include chloro or bromo groups, and preferred leaving groups for compounds of formula V in which X^{1a} is -Q-X² and Q is a single bond include chloro or bromo groups, —B(OH)₂, 4,4,5,5-tetramethyl-1,3,2-dioxaborolan-2-yl, 9-borabicyclo-[3.3.1]nonane (9-BBN) or —Sn(alkyl)₃. This reaction may be performed, for example in the presence of a suitable catalyst system, e.g. a metal (or a salt or complex thereof) such as CuI, Pd/C, PdCl₂, Pd(OAc)₂, Pd(Ph₃P)₂Cl₂, Pd(Ph₃P)₄, Pd₂(dba)₃ or NiCl₂ and a ligand such as i-Bu₃P, (C₆H₁₁)₃P, Ph₃P, AsPh₃, P(o-Tol)₃, 1,2-bis(diphenylphosphino)ethane, 2,2′-bis(di-tert-butylphosphino)-1,1′-biphenyl, 2,2′-bis(diphenylphosphino)-1,1′-bi-naphthyl, 1,1′-bis(diphenylphosphino-ferrocene), 1,3-bis(diphenyl-phosphino)propane, xantphos, or a mixture thereof, together with a suitable base such as, Na₂CO₃, K₃PO₄, Cs₂CO₃, NaOH, KOH, K₂CO₃, CsF, Et₃N, (i-Pr)₂NEt, t-BuONa or i-BuOK (or mixtures thereof) in a suitable solvent such as dioxane, toluene, ethanol, dimethylformamide, ethylene glycol dimethyl ether, water, dimethylsulfoxide, acetonitrile, dimethylacetamide, N-methylpyrrolidinone, tetrahydrofuran or mixtures thereof. The reaction may also be carried out for example at room temperature or above (e.g. at a high temperature such as the reflux temperature of the solvent system) or using microwave irradiation. The skilled person will appreciate that certain compounds of formula IV (in particular those in which L¹ represents chloro, bromo or iodo) are also compounds of formula I and therefore compounds of the invention. In the case where X^{1a} represents Q-X² and:
(I) Q represents C₂₋₈ alkenylene or C₂₋₈ heteroalkenylene and X² is as hereinbefore defined; or
(II) Q represents a single bond and X² represents C₂₋₈ alkenyl, C₂₋₈ heteroalkenyl or heterocycloalkenyl,
   and, in each case, the double bond is between the atoms that are α and β to L², the skilled person will appreciate that the double bond may migrate on formation of the compound of formula I to form a double bond that is between the atoms that are β and γ to the indole ring;
   (iia) for compounds of formula I in which X¹ represents —C(O)N(H)C(═NR^{9c})N(R^{10d})R^{9d}, —C(O)N(H)CN or —C(O)N(H)S(O)₂R¹¹, reaction of either a compound of formula I in which X¹ represents H, or a compound of formula IV in which the L¹ group is activated (for example as described hereinafter in respect of process (x) and so forming, for example, an —Mg-halide or a -L¹ group), with a compound of formula VA,

R^{za}—N═C═O VA

wherein R¹ represents —C(═NR^{9c})N(R^{10d})R^{9d}, —CN or —S(O)₂R¹¹, followed by quenching with a suitable proton source (e.g. water or aqueous, saturated NH₄Cl solution). This reaction may be performed in the presence of a suitable solvent, such as a polar aprotic solvent (e.g. tetrahydrofuran or diethyl ether), at sub-ambient temperatures (e.g. 0° C. to −78° C.) under an inert atmosphere;
(iii) for compounds of formula I in which X¹ represents -Q-X² and Q represents —C(O)—, reaction of a compound of formula I in which X¹ represents H with a compound of formula V in which X^{1a} represents -Q-X², Q represents —C(O)— and L² represents a suitable leaving group such as chloro or bromo, —N(C₁₋₆ alkyl)₂ (e.g. —N(CH₃)₂) or a carboxylate group such as —O—C(O)—X^{2y} in which X^{2y} represents X² or H. In the latter case, X^{2y} and X² are preferably the same, or X^{2y} represents e.g. H, CH₃ or CF₃. This reaction may be performed under suitable conditions known to those skilled in the art, for example in the presence of a suitable Lewis acid (e.g. AlCl₃ or FeCl₃). Reaction of a compound of formula V in which L² represents —N(C₁₋₆ alkyl)₂, X^{1a} represents -Q-X² and X² represents optionally substituted aryl (e.g. phenyl) or heteroaryl may be performed in the presence of a reagent such as POCl₃, for example under reaction conditions described in Bioorg. Med. Chem. Lett., 14, 4741-4745 (2004). The skilled person will appreciate that in the latter instance, POCl₃ may convert the compound of formula V into one in which L² represents chloro and/or X^{1a} represents -Q-X² in which Q represents a derivative of —C(O)— (e.g. an iminium derivative), which group may be transformed back to a —C(O)— group before or after reaction with the compound of formula I in which X¹ represents H;
(iv) for compounds of formula I in which X¹ represents —N(R^{9k})-J-R^{10k} or -Q-X² in which Q represents —O—, —S—, C₂₋₈ alkynylene or C₂₋₈ heteroalkylene in which latter two groups the triple bond is adjacent to the indole ring of formula I, reaction of a compound of formula IV as hereinbefore defined with a compound of formula VI,

X^{1b}H VI

in which X^{1b} represents —N(R^{9k})-J-R^{10k} or -Q-X² in which Q represents —O—, —S—, C₂₋₈ alkynylene or C₂₋₈ heteroalkynylene, and R^{9k}, J, R^{10k} and X² are as hereinbefore defined, for example under reaction conditions as hereinbefore described in respect of either process (i) or (ii) above;
(v) for compounds of formula I in which X¹ represents -Q-X² and Q represents —S—, reaction of a compound of formula I in which X¹ represents H, with a compound of formula VI in which X^{1b} represents -Q-X², Q represents —S— and X² is as hereinbefore defined, for example in the presence of N-chlorosuccinimide and a suitable solvent (e.g. dichloromethane), e.g. as described in inter alia Org. Lett., 819-821 (2004). Alternatively, reaction with a compound of formula VI in which X^{1b} represents -Q-X², Q represents —S— and X² represents an optionally substituted aryl(phenyl) or heteroaryl (e.g. 2-pyridyl) group, may be performed in the presence of PIFA (PhI(OC(O)CF₃)₂) in a suitable solvent such as (CF₃)₂CHOH. Introduction of such an —S—X² group is described in inter alia Bioorg. Med. Chem. Lett., 14, 4741-4745 (2004);
(vi) for compounds of formula I in which X¹ represents -Q-X² and Q represents —S(O)— or —S(O)₂—, oxidation of a corresponding compound of formula I in which Q represents —S— under appropriate oxidation conditions, which will be known to those skilled in the art;
(vii) for compounds of formula I in which X¹ represents -Q-X², X² represents C₁₋₈ alkyl substituted by G¹, G¹ represents -A¹-R^{12a}, A¹ represents —N(R^{13a})A⁴- and A⁴ is a single bond (provided that Q represents a single bond when X² represents substituted C₁ alkyl), reaction of a compound of formula VIII,

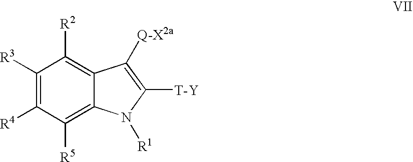

wherein X^{2a} represents a C₁₋₈ alkyl group substituted by a -Z¹ group in which Z¹ represents ═O, Q is as hereinbefore defined, provided that it represents a single bond when X^{2a} represents C₁ alkyl substituted by ═O (i.e.—CHO), and R¹, R², R³, R⁴, R⁵, T and Y are as hereinbefore defined under reductive amination conditions in the presence of a compound of formula VIII,

R^{12a}(R^{13a})NH VIII

wherein R^{12a} and R^{13a} are as hereinbefore defined, under conditions well known to those skilled in the art;
(viia) for compounds of formula I in which X¹ represents -Q-X², Q represents a single bond, X² represents methyl substituted by G¹, G¹ represents -A¹-R^{12a}, A¹ represents —N(R^{13a})A⁴-, A⁴ is a single bond and R^{12a} and R^{13a} are preferably methyl, reaction of a corresponding compound of formula I in which X¹ represents H, with a mixture of formaldehyde (or equivalent reagent) and a compound of formula VIII as hereinbefore defined (e.g. in which R^{12a} and R^{13a} represent methyl), for example in the presence of solvent such as a mixture of acetic acid and water, under e.g. standard Mannich reaction conditions known to those skilled in the art;
(viii) for compounds of formula I in which X¹ represents -Q-X², Q represents a single bond and X² represents optionally substituted C₂₋₈ alkenyl (in which a point of unsaturation is between the carbon atoms that are É and é to the indole ring), reaction of a corresponding compound of formula I in which X¹ represents halo (e.g. iodo) with a compound of formula IXA,

H₂C═C(H)X^{2b} IXA

or, depending upon the geometry of the double bond, reaction of a compound of formula VII in which Q represents a single bond and X^{2a} represents —CHO with either a compound of formula IXB,

(EtO)₂P(O)CH₂X^{2b} IXB

or the like, or a compound of formula IXC,

(Ph)₃P═C(H)X^{2b} IXC

or the like, wherein, in each case, X^{2b} represents H, X³, G¹ or C₁₋₆ alkyl optionally substituted with one of more substituents selected from X³, G¹ and/or Z¹ and X³, G¹ and Z¹ are as hereinbefore defined, for example, in the case of a reaction of a compound of formula IV with compound of formula IXA, in the presence of an appropriate catalyst (such as PdCl₂(PPh₃)₂), a suitable base (e.g. NaOAc and/or triethylamine) and an organic solvent (e.g. DMF) and, in the case of reaction of a compound of formula VII with either a compound of formula IXB, or IXC, under standard Horner-Wadsworth-Emmons, or Wittig, reaction conditions, respectively;
(ix) for compounds of formula I in which X¹ represents -Q-X² and X² represents optionally substituted, saturated C₂₋₈ alkyl, saturated cycloalkyl, saturated C₂₋₈ heterocycloalkyl, saturated heterocycloalkyl, C₂₋₈ alkenyl, cycloalkenyl, C₂₋₈ heterocycloalkenyl or heterocycloalkenyl, reduction (e.g. hydrogenation) of a corresponding compound of formula I in which X² represents optionally substituted C₂₋₈ alkenyl, cycloalkenyl, C₂₋₈ heterocycloalkenyl, heterocycloalkenyl, C₂₋₈ alkynyl, cycloalkynyl, C₂₋₈ heterocycloalkynyl or heterocycloalkynyl (as appropriate) under conditions that are known to those skilled in the art. For example, in the case where an alkynyl group is converted to a alkenyl group, in the presence of an appropriate poisoned catalyst (e.g. Lindlar's catalyst);
(x) for compounds of formula I in which D represents a single bond, —C(O)—, —C(R⁷)(R⁸)—, C₂₋₄ alkylene or —S(O)₂—, reaction of a compound of formula X,

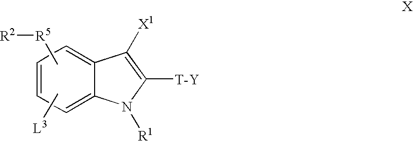

wherein L³ represents L¹ or L² as hereinbefore defined, which group is attached to one or more of the carbon atoms of the benzenoid ring of the indole, R²—R⁵ represents whichever of the three other substituents on the benzenoid ring, i.e. R², R³, R⁴ and R⁵, are already present in that ring, and X¹, R¹, R², R³, R⁴, R⁵, T and Y are as hereinbefore defined, with a compound of formula XI,

E-D^{a}-L⁴ XI

wherein D^{a} represents a single bond, —C(O)—, —C(R⁷)(R⁸)—, C₂₋₄ alkylene or —S(O)₂—, L⁴ represents L¹ (when L³ is L²) or L² (when L³ is L¹), and L¹, L², E, R⁷ and R⁸ are as hereinbefore defined. For example, when D^{a} represents a single bond, —C(O)— or C₂₋₄ alkylene, the reaction may be performed for example under similar conditions to those described hereinbefore in respect of process step (ii) above. Further, when D^{a} represents —C(O)—, —C(R⁷)(R⁸)—, C₂₋₄ alkylene or —S(O)₂—, the reaction may be performed by first activating the compound of formula X. The skilled person will appreciate that compounds of formula X may be activated when L³ represents halo, by:
(I) forming the corresponding Grignard reagent under standard conditions known to those skilled in the art (e.g. employing magnesium or a suitable reagent such as a mixture of C₁₋₆ alkyl-Mg-halide and ZnCl₂ or LiCl), followed by reaction with a compound of formula XI, optionally in the presence of a catalyst (e.g. FeCl₃) under conditions known to those skilled in the art; or
(II) forming the corresponding lithiated compound under halogen-lithium exchange reaction conditions known to those skilled in the art (e.g. employing n-BuLi or t-BuLi in the presence of a suitable solvent (e.g. a polar aprotic solvent such as THF)), followed by reaction with a compound of formula XI.

The skilled person will also appreciate that the magnesium of the Grignard reagent or the lithium of the lithiated species may be exchanged to a different metal (i.e. a transmetallation reaction may be performed), for example to zinc (e.g. using ZnCl₂) and the intermediate so formed may then be subjected to reaction with a compound of formula XI under conditions known to those skilled in the art, for example such as those described hereinbefore in respect of process (ii) above;

(xi) for compounds of formula I in which D represents —S—, —O— or C₂₋₄ alkynylene in which the triple bond is adjacent to E, reaction of a compound of formula X as hereinbefore defined in which L³ represents L² as hereinbefore defined (for example —B(OH)₂) with a compound of formula XII,

E-D^{b}-H XII

wherein D^{b} represents —S—, —O— or C₂₋₄ alkynylene in which the triple bond is adjacent to E and E is as hereinbefore defined. Such reactions may be performed under similar conditions to those described hereinbefore in respect of process step (ii) above, for example in the presence of a suitable catalyst system, such as Cu(OAc)₂, a suitable base, such as triethylamine or pyridine, and an appropriate organic solvent, such as DMF or dichloromethane;
(xii) for compounds of formula I in which D represents —S(O)— or —S(O)₂—, oxidation of a corresponding compound of formula I in which D represents —S— under appropriate oxidation conditions, which will be well known to those skilled in the art;
(xiii) for compounds of formula I in which D represents —O— or —S—, reaction of a compound of formula XIII,

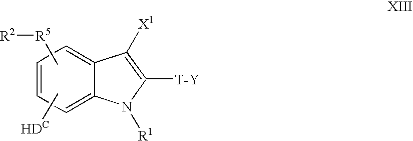

wherein the -D^{c}-H group is attached to one or more of the carbon atoms of the benzenoid ring of the indole, D^{c} represents —O— or —S— and X¹, R¹, R²—R⁵, T and Y are as hereinbefore defined, with a compound of formula XIV,

E-L² XIV

wherein L² is as hereinbefore defined (for example —B(OH)₂, chloro, bromo or iodo) and E is as hereinbefore defined, for example under conditions such as those described hereinbefore in respect of process step (ii) above;
(xiv) for compounds of formula I in which X¹ represents —N(R^{9k})-J-R^{10k}, reaction of a compound of formula XV,

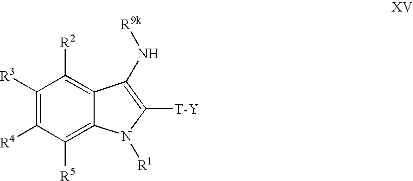

wherein R¹, R², R³, R⁴, R⁵, T, Y and R^{9k} are as hereinbefore defined, with a compound of formula XVI,

R^{10k}-J-L¹ XVI

wherein J, R^{10k} and L¹ are as hereinbefore defined, for example at around room temperature or above (e.g. up to 60-70° C.) in the presence of a suitable base (e.g. pyrrolidinopyridine, pyridine, triethylamine, tributylamine, trimethylamine, dimethylaminopyridine, diisopropylamine, 1,8-diazabicyclo[5.4.0]undec-7-ene, sodium hydroxide, or mixtures thereof) and an appropriate solvent (e.g. pyridine, dichloromethane, chloroform, tetrahydrofuran, dimethylformamide, dimethylsulfoxide, water, triethylamine or mixtures thereof) and, in the case of biphasic reaction conditions, optionally in the presence of a phase transfer catalyst;
(xv) for compounds of formula I in which X¹ represents —N(R^{9k})-J-R^{10k}, J represents a single bond and R^{10k} represents a C₁₋₈ alkyl group, reduction of a corresponding compound of formula I, in which J represents —C(O)— and R^{10k} represents H or a C₁₋₇ alkyl group, in the presence of a suitable reducing agent. A suitable reducing agent may be an appropriate reagent that reduces the amide group to the amine group in the presence of other functional groups (for example an ester or a carboxylic acid). Suitable reducing agents include borane and other reagents known to the skilled person;
(xvi) for compounds of formula I in which X¹ represents halo, reaction of a compound of formula I wherein X¹ represents H, with a reagent or mixture of reagents known to be a source of halide atoms. For example, for bromide atoms, N-bromosuccinimide, bromine or 1,2-dibromotetrachloroethane may be employed, for iodide atoms, iodine, diiodoethane, diiodotetrachloroethane or a mixture of NaI or KI and N-chlorosuccinimide may be employed, for chloride atoms, N-chlorosuccinimide may be employed and for fluoride atoms, 1-(chloromethyl)-4-fluoro-1,4-diazoniabicyclo[2.2.2]octane bis(tetrafluoroborate), 1-fluoropyridinium triflate, xenon difluoride, CF₃OF or perchloryl fluoride may be employed. This reaction may be carried out in a suitable solvent (e.g. acetone, benzene or dioxane) under conditions known to the skilled person;
(xvii) for compounds of formula I in which T represents optionally substituted, saturated C₂₋₈ alkylene, saturated cycloalkylene, saturated C₂₋₈ heteroalkylene, saturated heterocycloalkylene, C₂₋₈ alkenylene, cycloalkenylene, C₂₋₈ heteroalkenylene or heterocycloalkenylene, reduction (e.g. hydrogenation) of a corresponding compound of formula I in which T represents optionally substituted C₂₋₈ alkenylene, cycloalkenylene, C₂₋₈ heteroalkenylene, heterocycloalkenylene, C₂₋₈ alkynylene, cycloalkynylene, C₂₋₈ heteroalkynylene or heterocycloalkynylene (as appropriate) under conditions that are known to those skilled in the art;
(xviii) for compounds of formula I in which X¹ represents -Q-X² and Q represents —O—, reaction of a compound of formula XVII,

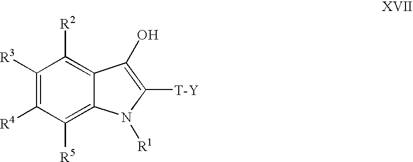

wherein R¹, R², R³, R⁴, R⁵, T and Y are as hereinbefore defined, with a compound of formula XVIII,

X²L⁷ XVIII

wherein L⁷ represents a suitable leaving group such as a halo or sulfonate group and X² is as hereinbefore defined, for example in the presence of a base or under reaction conditions such as those described hereinbefore in respect of process (xiii) above;

(xix) reaction of a compound of formula XIX,

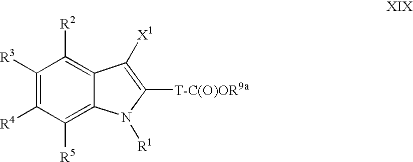

wherein R¹, R², R³, R⁴, R⁵, T, X¹ and R^{9a} are as hereinbefore defined, with a compound of formula XX,

R²⁵(R²⁶)NH XX

wherein R²⁵ and R²⁶ represent, in the case of a compound of formula I in which Y represents:
(1) —C(O)N(R^{10b})R^{9b}, R^{9b} and R^{10b};
(2) —C(O)N(H)C(═NR^{9c})N(R^{10d})R^{9d}, —C(═NR^{9c})N(R^{10d})R^{9d} and H;
(3) —C(O)N(H)CN, —CN and H; or
(4) —C(O)N(H)S(O)₂R¹¹, —S(O)₂R¹¹ and H,
   respectively, and R^{9a} to R^{9d}, R^{10b}, R^{10d} and R¹¹ are as hereinbefore defined under standard conditions. For example, the reaction may be performed in the presence of a suitable coupling reagent ((e.g. 1,1′-carbonyldiimidazole, N,N′-dicyclohexylcarbodiimide, 1-(3-dimethylaminopropyl)-3-ethylcarbodiimide (or hydrochloride thereof), N,N′-disuccinimidyl carbonate, benzotriazol-1-yloxytris(dimethylamino)phosphonium hexafluorophosphate, 2-(1H-benzotriazol-1-yl)-1,1,3,3-tetramethyluronium hexafluorophosphate, benzotriazol-1-yloxytrispyrrolidinophosphonium hexafluorophosphate, bromo-tris-pyrrolidinophosphonium hexafluorophosphate, 2-(1H-benzotriazol-1-yl)-1,1,3,3-tetramethyluronium tetrafluorocarbonate, 1-cyclohexylcarbodiimide-3-propyloxymethyl polystyrene, O-(7-azabenzotriazol-1-yl)-N,N,N,N′-tetramethyluronium hexafluorophosphate or O-benzotriazol-1-yl-N,N,N′,N′-tetramethyluronium tetrafluoroborate), and/or a suitable base (e.g. sodium hydride, sodium bicarbonate, potassium carbonate, pyrrolidinopyridine, pyridine, triethylamine, tributylamine, trimethylamine, dimethylaminopyridine, diisopropylamine, diisopropylethylamine, 1,8-diazabicyclo[5.4.0]undec-7-ene, sodium hydroxide, N-ethyldiisopropylamine, N-(methylpolystyrene)-4-(methylamino)pyridine, potassium bis(trimethylsilyl)amide, sodium bis(trimethylsilyl)amide, potassium teri-butoxide, lithium diisopropylamide, lithium 2,2,6,6-tetramethylpiperidine, butyllithium (e.g. n-, s- or t-butyllithium) or mixtures thereof) and an appropriate solvent (e.g. tetrahydrofuran, pyridine, toluene, dichloromethane, chloroform, acetonitrile, dimethylformamide, dimethylsulfoxide, water, triethylamine or mixtures thereof). Alternatively an azodicarboxylate may be employed under Mitsunobo conditions known to those skilled in the art. The skilled person will also appreciate that it may be convenient or necessary to first convert the acid or ester compound of formula I to a corresponding acid halide prior to reaction with the compound of formula XX. Such conversions may be performed in the presence of a suitable reagent (e.g. oxalyl chloride, thionyl chloride, etc) optionally in the presence of an appropriate solvent (e.g. dichloromethane, THF, toluene or benzene) and a suitable catalyst (e.g. DMF), resulting in the formation of the respective acyl chloride. The skilled person will appreciate that when compounds of formula XX are liquid in nature, they may serve as both solvent and reactant in this reaction. An alternative way of performing this step for compounds of formula I in which Y represents —C(O)N(R^{10b})R^{9b}, includes the reaction of a compound of formula XIX in which R^{9a} is other than H (e.g. ethyl) with a compound of formula XX, in the presence of, e.g. trimethylaluminium, for example in an inert atmosphere and in the presence of a suitable solvent (e.g. dichloromethane);
   (xx) for compounds of formula I in which X¹ is as hereinbefore defined, and is preferably other than —C(O)N(H)C(═NR^{9c})N(R^{10d})R^{9d}, provided that, when X¹ represents —C(O)OR^{9a}, —C(O)N(R^{10b})R^{9b}, —S(O)₂N(R^{10b})R^{9b}, —C(O)N(H)C(═NR^{9c})N(R^{10d})R^{9d}—S(O)₃R^{9e}, —P(O)(OR⁹ⁱ)₂, —P(O)(OR^{9g})N(R¹)R^{9b}, —P(O)(N(R¹⁰ⁱ)R⁹ⁱ)₂, or —B(OR^{9j})₂, R^{9a} to R^{9g}, R⁹ⁱ, R^{9j}, R^{10b}, R^{10d}, R^{10h} and R¹⁰ⁱ are other than H, reaction of a compound of formula XXI,

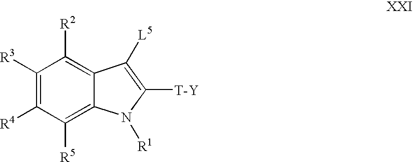

wherein L⁵ represents an appropriate alkali metal group (e.g. sodium, potassium or, especially, lithium), a —Mg-halide, a zinc-based group or a suitable leaving group such as halo or —B(OH)₂, or a protected derivative thereof (the skilled person will appreciate that the compound of formula XXI in which L⁵ represents an alkali metal (e.g. lithium), a Mg-halide or a zinc-based group may be prepared from a corresponding compound of formula XXI in which L⁵ represents halo, for example under conditions such as those hereinbefore described in respect of preparation of compounds of formula I (process step (x) above)), and T, Y, R¹, R², R³, R⁴ and R⁵ are as hereinbefore defined, with a compound of formula XXI1,

L⁶-X^{1b} XXII

wherein X^{1b} represents X¹ (and X¹ is preferably other than —C(O)N(H)C(═NR^{9c})N(R^{10d})R^{9d}), provided that when X¹ represents —C(O)OR^{9a}, —C(O)N(R^{10b})R^{9b}, —S(O)₂N(R^{10b})R^{9b}, —C(O)N(H)C(═NR^{9c})N(R^{10d})R^{9d}, —S(O)₃R^{9e}, —P(O)(OR⁹)₂, —P(O)(OR^{9g})N(R^{10h})R^{9h}, —P(O)(N(R¹⁰ⁱ)R⁹ⁱ)₂, or —B(OR^{9j})₂, R^{9a} to R^{9g}, R⁹ⁱ, R^{9j}, R^{10b}, R^{10d}, R^{10h} and R¹⁰ⁱ are other than H, or a protected derivative thereof, and L⁶ represents a suitable leaving group known to those skilled in the art, such as halo (especially chloro or bromo), for example when X^{1b} represents —C(O)OR^{9a}, or —S(O)₃R^{9e}, or C₁₋₃ alkoxy, for example when X^{1b} represents —B(OR^{9j})₂. The reaction may be performed under similar reaction conditions to those described hereinbefore in respect of process (x) above, followed by (if necessary) deprotection under standard conditions. The skilled person will also appreciate that L⁵ and L⁶ (when they both represent leaving groups) will be mutually compatible in a similar manner to the L¹ and L² groups described hereinbefore in process step (ii) above;

(xxi) for compounds of formula I in which X¹ represents —C(O)OR^{9a} and R^{9a} represents H, reaction of a compound of formula XXI in which L⁵ represents either:
(I) an alkali metal (for example as defined in respect of process step (x) above); or
(II) —Mg-halide,
   with carbon dioxide, followed by acidification under standard conditions known to those skilled in the art, for example, in the presence of aqueous hydrochloric acid;
   (xxii) for compounds of formula I in which X¹ represents —C(O)OR^{9a} or —C(O)N(R^{10b})R^{9b}, reaction of a corresponding compound of formula XXI in which L⁵ is a suitable leaving group known to those skilled in the art (such as a sulfonate group (e.g. a triflate) or, preferably, a halo (e.g. bromo or iodo) group) with CO (or a reagent that is a suitable source of CO (e.g. Mo(CO)₆ or CO₂(CO)₈)), in the presence of a compound corresponding to a compound of formula XXIIA,

R^{9a}OH XXIIA

wherein R^{9a} is as hereinbefore defined, or a compound of formula XX as hereinbefore defined in which R²⁵ and R²⁶ represent R^{9b} and R^{10b} respectively, and an appropriate catalyst system (e.g. a palladium catalyst such as one described hereinbefore in respect of process step (ii)) under conditions known to those skilled in the art;
(xxiii) for compounds of formula I in which X¹ represents —B(OR^{9j})₂ and R^{9j} represents H, reaction of a compound of formula XXI as hereinbefore defined with boronic acid or a protected derivative thereof (e.g. bis(pinacolato)diboron or triethyl borate) and an appropriate catalyst system (e.g. a palladium catalyst such as one described hereinbefore in respect of process step (ii)) under conditions known to those skilled in the art, followed by (if necessary) deprotection under standard conditions;
(xxiv) for compounds of formula I in which X¹ represents —S(O)₃R^{9e} or —S(O)₂N(R^{10b})R^{9b}; reaction of a compound of formula XXI as hereinbefore defined with:
(A) for compounds of formula I in which X¹ represents —S(O)₃R^{9e}, and R^{9e} represents H, either SO₃ (or a suitable source of SO₃ such as a SO₃*pyridine or SO₃*Et₃N complex) or with SO₂ followed by treatment with N-chlorosuccinimide and then hydrolysis. Alternatively, a compound of formula XXI may be reacted with a protected sulfide, followed by deprotection and oxidation, or a compound of formula XXI may be reacted with chlorosulfonic acid (CIS(O)₂OH) followed by hydrolysis; (B) for compounds of formula I in which X¹ represents —S(O)₃R^{9e}, and R^{9e} is other than H, chlorosulfonic acid followed by reaction with a compound of formula XXIII as defined hereinafter in which R^{9za} represents R^{9e};
(C) for compounds of formula I in which X¹ represents —S(O)₂N(R^{10b})R^{9b}, chlorosulfonic acid followed by reaction with a compound of formula XX as defined hereinbefore, all under standard conditions;
   (xxv) for compounds of formula I in which Q represents optionally substituted C₂₋₈ alkenylene or C₂₋₈ heteroalkenylene (in which a point of unsaturation is between the carbon atoms that are É and é to the indole ring), reaction of a compound of formula VII in which Q represents a single bond and X^{2a} represents —CHO with a compound of formula XXIIB,

(Ph)₃P═C(H)-Q^{c}-X¹ XXIIB

or the like (e.g. the corresponding Horner-Wadsworth-Emmons reagent), wherein Q^{c} represents a single bond or optionally substituted C₁₋₆ alkylene or C₂₋₆ heteroalkylene, X¹ is as hereinbefore defined, for example under standard Wittig reaction conditions, e.g. in the presence of a suitable organic solvent (e.g. DMF);
(xxvi) for compounds of formula I in which Q represents optionally substituted, saturated C₂₋₈ alkylene, saturated cycloalkylene, saturated C₂₋₈ heteroalkylene, saturated heterocycloalkylene, C₂₋₈ alkenylene, cycloalkenylene, C₂₋₈ heteroalkenylene or heterocycloalkenylene, reduction (e.g. hydrogenation) of a corresponding compound of formula I in which Q represents optionally substituted C₂₋₈ alkenylene, cycloalkenylene, C₂₋₈ heteroalkenylene, heterocycloalkenylene, C₂₋₈ alkynylene, cycloalkynylene, C₂₋₈ heteroalkynylene or heterocycloalkynylene as appropriate) under conditions that are known to those skilled in the art;
(xxvii) for compounds of formula I in which X¹ represents —C(O)OR^{9a}, —S(O)₃R^{9e}, —P(O)(OR^{9f})₂ or —B(OR^{9j})₂, in which R^{9a}, R^{9e}, R^{9f} and R⁹ⁱ represent H, hydrolysis of a corresponding compound of formula I in which R^{9a}, R^{9e}, R^{9f} and R⁹ⁱ do not represent H, or, for compounds of formula I in which X¹ represents —C(O)OR^{9a} or —P(O)(OR^{9f})₂, in which R^{9a} and R^{9f} represent H, a corresponding compound of formula I in which X¹ represents —C(O)N(H)S(O)₂R¹¹, —P(O)(OR^{9g})N(R^{10h})R^{9h} or —P(O)(N(R¹⁰ⁱ)R⁹ⁱ)₂ (as appropriate), all under standard conditions;
(xxviii) for compounds of formula I in which X¹ represents —C(O)OR^{9a}, —S(O)₃R^{9e}, —P(O)(OR^{9f})₂, —P(O)(OR^{9g})N(R^{10h})R^{9h} or —B(OR^{9j})₂ and R^{9a}, R^{9e}, R^{f}, and R^{9j} (i.e. those R⁹ groups attached to an oxygen atom) do not represent H:
   (A) esterification of a corresponding compound of formula I in which R^{9a},
   R^{9e}, R^{9f}, R^{9g} and R^{9j} represent H; or
   (B) trans-esterification of a corresponding compound of formula I in which R^{9a}, R^{9e}, R^{9r}, R^{9g} and R^{9j} do not represent H (and do not represent the same value of the corresponding R^{9a}, R^{9e}, R^{9f}, R^{9g} and R^{9j} group in the compound of formula I to be prepared),
      under standard conditions in the presence of the appropriate alcohol of formula XXIII,

R^{9za}OH XXIII

in which R^{9za} represents R^{9a}, R^{9e}, R^{9f}, R^{9g} or R^{9j} provided that none of those R⁹ groups represent H;

(xxix) for compounds of formula I in which Q represents a C₁ alkylene group substituted with G¹, in which G¹ represents -A¹-R^{12a}, A¹ represents —C(O)A²-, A² represents a single bond and R^{12a} represents H, and X² represents —C(O)OR^{9a}, in which R^{9a} is other than H, reaction of a corresponding compound of formula I in which the C₁ alkylene group that Q represents is unsubstituted with C₁₋₆ alkyl (e.g. ethyl) formate in the presence of a suitable base (sodium ethoxide), for example under similar conditions to those described in Bioorg. Med. Chem. Lett., 13, 2709 (2003);
(xxx) for compounds of formula I in which X¹ represents —C(O)N(R^{10b})R^{9b}, —C(O)N(H)C(═NR^{9c})N(R^{10d})R^{9d}, —C(O)N(H)CN or —C(O)N(H)S(O)₂R¹¹ reaction of a corresponding compound of formula I in which X¹ represents —C(O)OR^{9a} with a compound of formula XX as hereinbefore defined under standard conditions, for example such as those described hereinbefore in respect of process (xix) above;
(xxxi) for compounds of formula I in which X¹ represents -Q-X², Q represents a single bond and X² represents C₁₋₈ alkyl or heterocycloalkyl substituted (x to the indole ring by a G¹ substituent in which G¹ represents -A¹-R^{12a}, A¹ represents —OA⁵-, A⁵ represents a single bond and R¹² represents H, reaction of a corresponding compound of formula I in which X¹ represents H with a compound corresponding to a compound of formula VI, but in which X^{1b} represents -Q-X², Q represents a single bond and X² represents C₁₋₈ alkyl or heterocycloalkyl, both of which groups are substituted by a Z¹ group in which Z¹ represents ═O, under conditions known to those skilled in the art, for example optionally in the presence of an acid, such as a protic acid or an appropriate Lewis acid. Such substitutions are described in inter alia Bioorg. Med. Chem. Lett., 14, 4741-4745 (2004) and Tetrahedron Leti. 34, 1529 (1993);
(xxxii) for compounds of formula I in which X¹ represents -Q-X², Q represents a single bond and X² represents C₂₋₈ alkyl substituted (e.g. cc to the indole ring) by a G¹ substituent in which G¹ represents -A¹-R^{12a}, A¹ represents —OA⁵-, A⁵ represents a single bond and R^{12a} represents H, reaction of a corresponding compound of formula I in which X² represents C₁₋₇ alkyl substituted (e.g. aX to the indole ring) by a Z¹ group in which Z¹ represents ═O, with the corresponding Grignard reagent derivative of a compound of formula V in which L² represents chloro, bromo or iodo, X^{1a} represents -Q-X², Q is a single bond and X² represents C₁₋₇ alkyl, under conditions known to those skilled in the art;
(xxxiii) for compounds of formula I in which X¹ represents -Q-X², Q represents a single bond, and X² represents C₁₋₈ alkyl or heterocycloalkyl, both of which are unsubstituted in the position (x to the indole ring, reduction of a corresponding compound of formula I in which X² represents C₁₋₈ alkyl substituted α to the indole ring by a G¹ substituent in which G¹ represents -A¹-R^{12a}, A¹ represents —OA⁵-, A⁵ represents a single bond and R^{12a} represents H, in the presence of a suitable reducing agent such as a mixture of triethyl silane and a protic acid (e.g. CF₃COOH) or a Lewis acid (e.g. (CH₃)₃SiOS(O)₂CF₃) for example under conditions described in inter alia Bioorg. Med. Chem. Lett., 14, 4741-4745 (2004);
(xxxiv) for compounds of formula I in which X¹ represents -Q-X², Q represents a single bond and X² represents C₁₋₈ alkyl or heterocycloalkyl, neither of which are substituted by Z¹ in which Z¹ represents ═O, reduction of a corresponding compound of formula I in which X² represents C₁₋₈ alkyl or heterocycloalkyl, which groups are substituted by one or more Z¹ groups in which Z¹ represents ═O under conditions known to those skilled in the art, for example employing NaBH₄ in the presence of an acid (e.g. CH₃COOH or CF₃COOH), Wolff-Kishner reduction conditions (i.e. by conversion of the carbonyl group to a hydrazone, followed by base induced elimination) or by conversion of the carbonyl to the thioacetal analogue (e.g. by reaction with a dithiane) followed by reduction with e.g. Raney nickel, all under reaction conditions known to those skilled in the art; or
(xxxv) for compounds of formula I in which X¹ represents —N(R^{9k})-J-R^{10k}, reaction of a compound of formula XVII as hereinbefore defined, with a compound of formula VI in which X^{1b} represents —N(R^{9k})-J-R^{10k} and R^{9k}, R^{10k} and J are as hereinbefore defined, for example under reaction conditions known to those skilled in the art (such as those described in Journal of Medicinal Chemistry 1996, Vol. 39, 4044 (e.g. in the presence of MgCl₂)).

Compounds of formula II may be prepared by:
(a) reaction of a compound of formula XXIV,

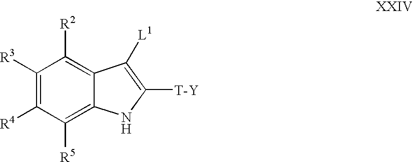

wherein L¹, R², R³, R⁴, R⁵, T and Y are as hereinbefore defined, with, for compounds of formula II in which X¹ represents:
(1) X¹ represents —C(O)OR^{9a}, —C(O)N(R^{10b})R^{9b}, —S(O)₂N(R^{10b})R^{9b}, —C(O)N(H)C(═NR^{9c})N(R^{10d})R^{9d}; —C(O)N(H)CN, —S(O)₃R^{9e}, —P(O)(OR^{9f})₂, —P(O)(OR^{9g})N(R^{10h})R^{9h}, —P(O)(N(R¹⁰ⁱ)R⁹ⁱ)₂, —B(OR^{9j})₂, —C(O)N(H)S(O)₂R¹¹ or -Q-X², in which Q is a single bond, —C(O)—, C₁₋₈ alkylene or C₂₋₈ alkylene, a compound of formula V as hereinbefore defined; or
(2) —N(R)-J-R^{10k} or -Q-X², in which Q represents —O—, —S—, C₂₋₈ alkynylene or C₂₋₈ heteroalkynylene in which latter two groups the triple bond is adjacent to the indole ring of formula II, a compound of formula VI as hereinbefore defined;
for example under reaction conditions similar to those described hereinbefore in respect of preparation of compounds of formula I (processes (ii) and (iv), respectively) above;
(bi) for compounds of formula II in which X¹ represents —C(O)N(H)C(═NR^{9c})N(R^{10d})R^{9d}, —C(O)N(H)CN or —C(O)N(H)S(O)₂R¹¹, reaction of either a compound of formula II (or a protected derivative thereof) in which X¹ represents H, or a compound of formula XXIV (or a protected derivative thereof) in which the L¹ group is activated (for example as described hereinbefore), with a compound of formula VA, as hereinbefore defined, for example under conditions such as those described hereinbefore in respect of preparation of compounds of formula I (process (iia) above);
(bii) for compounds of formula II in which X¹ represents -Q-X² and Q represents —C(O)—, reaction of a corresponding compound of formula II in which X¹ represents H with a compound of formula V in which X^{1a} represents -Q-X², Q represents —C(O)— and L² represents a suitable leaving group, for example under conditions such as those described in respect of preparation of compounds of formula I (process (iii)) above;
(c) for compounds of formula II in which X¹ represents -Q-X² and Q represents —S—, reaction of a corresponding compound of formula II in which X¹ represents H with a compound of formula VI in which X^{1b} represents -Q-X² and Q represents —S—, for example under conditions such as those described hereinbefore in respect of preparation of compounds of formula I (process (v)) above;
(d) for compounds of formula II in which X¹ represent -Q-X² and Q represents —S(O)— or —S(O)₂—, oxidation a corresponding compound of formula II in which Q represent —S—;
(e) for compounds of formula II in which X¹ represents -Q-X², X² represents C₁₋₈ alkyl substituted by G¹, G¹ represents -A¹-R^{12a}, A¹ represents —N(R^{13a})A⁴- and A⁴ is a single bond (provided that Q represents a single bond when X² represents substituted C₁ alkyl), reaction of a compound of formula XXV,

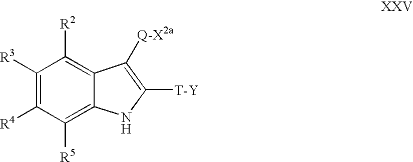

wherein Q, X^{2a}, R², R³, R⁴, R⁵, T and Y are as hereinbefore defined by reductive amination in the presence of a compound of formula VIII as hereinbefore defined;
(ea) for compounds of formula II in which X¹ represents -Q-X², Q represents a single bond, X² represents methyl substituted by G¹, G¹ represents -A1-R^{12a}, A¹ represents —N(R^{13a})A⁴-, A⁴ is a single bond and R^{12a} and R^{3a} are preferably methyl, reaction of a corresponding compound of formula II in which X¹ represents H, with a mixture of formaldehyde (or equivalent reagent) and a compound of formula VIII as hereinbefore defined, for example under reaction conditions similar to those described hereinbefore in respect of preparation of compounds of formula I (process (viia)) above;
(f) for compounds of formula II in which X¹ represents -Q-X², Q represents a single bond and X² represents optionally substituted C₂₋₈ alkenyl (in which a point of unsaturation is between the carbon atoms that are É and é to the indole ring), reaction of a corresponding compound of formula II in which X¹ represents halo (e.g. iodo) with a compound of formula IXA, or a compound of formula XXV in which Q represents a single bond and X^{2a} represents —CHO with a compound of formula IXB or a compound of formula IXC as hereinbefore defined, for example under reaction conditions similar to those described hereinbefore in respect of preparation of compounds of formula I (process (viii)) above;
(g) for compounds of formula II in which X¹ represents -Q-X² and X² represents optionally substituted, saturated C₂₋₈ alkyl, saturated cycloalkyl, saturated C₂₋₈ heterocycloalkyl, saturated heterocycloalkyl, C₂₋₈ alkenyl, cycloalkenyl, C₂₋₈ heterocycloalkenyl or heterocycloalkenyl, reduction (e.g. hydrogenation) of a corresponding compound of formula II in which X² represents optionally substituted C₂₋₈ alkenyl, cycloalkenyl, C₂₋₈ heterocycloalkenyl, heterocycloalkenyl, C₂₋₈ alkynyl, cycloalkynyl, C₂₋₈ heterocycloalkynyl or heterocycloalkynyl (as appropriate);
(h) for compounds of formula II in which D represents a single bond, —C(O)—, —C(R⁷)(R⁸)—, C₂₋₈ alkylene or —S(O)₂—, reaction of a compound of formula XXVI,

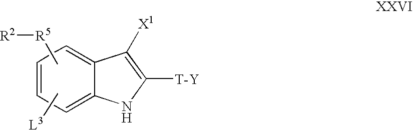

wherein X¹, L³, R²—R⁵, T and Y are as hereinbefore defined with a compound of formula XI as hereinbefore defined, for example under reaction conditions similar to those described hereinbefore in respect of preparation of compounds of formula I (process (x)) above;
(i) for compounds of formula II in which D represents —S—, —O— or C₂₋₄ alkynylene in which the triple bond is adjacent to E, reaction of a compound of formula XXVI as hereinbefore defined in which L³ represents L² as hereinbefore defined (for example —B(OH)₂) with a compound of formula XII as hereinbefore defined, for example under reaction conditions similar to those described hereinbefore in respect of preparation of compounds of formula I (process (xi)) above;
(j) for compounds of formula II in which D represents —S(O)— or —S(O)₂—, oxidation of a corresponding compound of formula II in which D represents —S—;
(k) for compounds of formula II in which D represents —O— or —S—, reaction of a compound of formula XXVII,

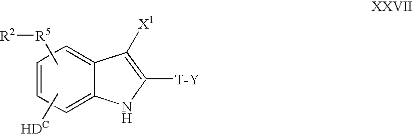

wherein D_{c}, X¹, R²—R⁵, T and Y are as hereinbefore defined, with a compound of formula XIV as hereinbefore defined;
(I) for compounds of formula II in which X¹ represents —N(R^{9k})-J-R^{10k}, reaction of a compound of formula XXVIII,

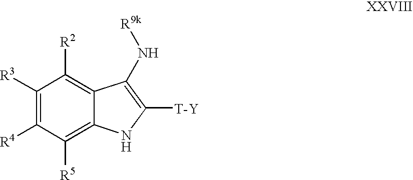

wherein R², R³, R⁴, R⁵, R^{9k}, T and Y are as hereinbefore defined with a compound of formula XVI as hereinbefore defined, for example under reaction conditions similar to those described hereinbefore in respect of preparation of compounds of formula I (process (xiv)) above;
(m) for compounds of formula II in which X¹ represents —N(R^{9k})-J-R^{10k}, J represents a single bond and R^{10k} represents a C₁₋₈ alkyl group, reduction of a corresponding compound of formula II, in which J represents —C(O)— and R^{10k} represents H or a C₁₋₇ alkyl group, for example under reaction conditions similar to those described hereinbefore in respect of preparation of compounds of formula I (process (xv)) above;
(n) for compounds of formula II in which X¹ represents halo, reaction of a compound of formula II wherein X¹ represents H, with a reagent or mixture of reagents known to be a source of halide atoms, for example under reaction conditions similar to those described hereinbefore in respect of preparation of compounds of formula I (process (xvi)) above;
(o) for compounds of formula II in which T represents optionally substituted, saturated C₂₋₈ alkylene, saturated cycloalkylene, saturated C₂₋₈ heteroalkylene, saturated heterocycloalkylene, C₂₋₈ alkenylene, cycloalkenylene, C₂₋₈ heteroalkenylene or heterocycloalkenylene, reduction (e.g. hydrogenation) of a corresponding compound of formula II in which T represents optionally substituted C₂₋₈ alkenylene, cycloalkenylene, C₂₋₈ heteroalkenylene, heterocycloalkenylene, C₂₋₈ alkynylene, cycloalkynylene, C₂₋₈ heteroalkynylene or heterocycloalkynylene (as appropriate);
(p) for compounds of formula II in which X¹ represents -Q-X² and Q represents —O—, reaction of a compound of formula XXIX,

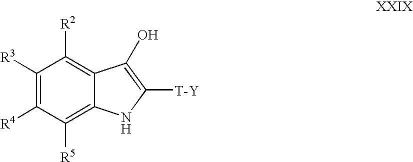

wherein R², R³, R⁴, R⁵, T and Y are as hereinbefore defined, with a compound of formula XVIII as hereinbefore defined, for example under reaction conditions described hereinbefore in respect of preparation of compounds of formula I (process (xviii)) above;
(q) reaction of a compound of formula XXX,

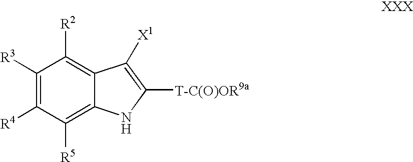

wherein R², R³, R⁴, R⁵, T, X¹ and R^{9a} are as hereinbefore defined, with a compound of formula XX as hereinbefore defined, for example under reaction conditions described in respect of preparation of compounds of formula I (process (xix)) above);
(r) for compounds of formula II in which X¹ is as hereinbefore defined, provided that when X¹ represents —C(O)OR^{9a}, —C(O)N(R^{10b})R^{9b}, —S(O)₂N(R^{10b})R^{9b}, —C(O)N(H)C(═NR^{9c})N(R^{10d})R^{9d}, S(O)₃R^{9e}, —P(O)(OR^{9f})₂, —P(O)(OR^{9g})N(R^{10h})R^{9h}, —P(O)((R¹⁰ⁱ)R⁹ⁱ)₂ or —B(OR^{9j})₂, R^{9a} to R^{9g}, R⁹ⁱ, R^{9j}, R^{10b}, R^{10d}, R^{10h} and R¹⁰ⁱ are other than H, reaction of a compound of formula XXXI,

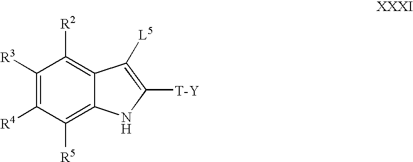

wherein L⁵, T, Y, R², R³, R⁴ and R⁵ are as hereinbefore defined, with a compound of formula XXII as hereinbefore defined, or a protected derivative thereof, for example under similar reaction conditions to those described hereinbefore in respect of process (xx) above, followed by (if necessary) deprotection;
(s) for compounds of formula II in which X¹ represents —C(O)OR^{9a} and R^{9a} represents H, reaction of a compound of formula XXXI in which L⁵ represents either an alkali metal or —Mg-halide with carbon dioxide, followed by acidification;
(t) for compounds of formula II in which X¹ represents, —C(O)OR^{9a}, reaction of a compound of formula XXXI in which L⁵ is a suitable leaving group with CO in the presence of a compound of formula XXIIA as hereinbefore defined, for example under conditions such as those described in respect of process (xxii) above;
(u) for compounds of formula II in which X¹ represents —B(OR^{9j})₂ and R^{9j} represents H, reaction of a compound of formula XXXI as hereinbefore defined with boronic acid or a protected derivative thereof (e.g. bis(pinacolato)diboron or triethyl borate), followed by (if necessary) deprotection;
(v) for compounds of formula II in which X¹ represents —S(O)₃R^{9e} or —S(O)₂N(R^{10b})R^{9b}, reaction of a compound of formula. XXXI as hereinbefore defined with:
   (A) for compounds of formula II in which X¹ represents —S(O)₃R^{9e}, and R^{9e} represents H, either SO₃ or with SO₂ followed by treatment with N-chlorosuccinimide and then hydrolysis,
   (B) for compounds of formula II in which X¹ represents —S(O)₃R^{9e}, and R^{9e} is other than H, chlorosulfonic acid followed by reaction with a compound of formula XXIII as defined hereinbefore in which R^{9za} represents R^{9e};
   (C) for compounds of formula II in which X¹ represents —S(O)₂N(R^{10b})R^{9b}, chlorosulfonic acid followed by reaction with a compound of formula XX as defined hereinbefore,
   all under standard conditions such as those described in respect of process (xxiv) above;
(w) for compounds of formula II in which Q represents optionally substituted C₂₋₈ alkenylene or C₂₋₈ heteroalkylene (in which a point of unsaturation is between the carbon atoms that are É and é to the indole ring), reaction of a compound of formula XXV, in which Q represents a single bond and X^{2a} represents —CHO with a compound of formula XXIIB as hereinbefore defined;
(x) for compounds of formula II in which Q represents an optionally substituted, saturated C₂₋₈ alkylene, saturated cycloalkylene, saturated C₂₋₈ heteroalkylene, saturated heterocycloalkylene, C₂₋₈ alkenylene, cycloalkenylene, C₂₋₈ heteroalkenylene or heterocycloalkenylene, reduction (e.g. hydrogenation) of a corresponding compound of formula II in which Q represents optionally substituted C₂₋₈ alkenylene, cycloalkenylene, C₂₋₈ heteroalkenylene, heterocycloalkenylene, C₂₋₈ alkynylene, cycloalkynylene, C₂₋₈ heteroalkynylene or heterocycloalkynylene (as appropriate);
(y) for compounds of formula II in which X¹ represents —C(O)OR^{9a}, —S(O)₃R^{9e}, —P(O)(OR^{9f})₂ or —B(OR^{9j})₂, in which R^{9a}, R^{9e}, R^{9f} and R^{9j} represent H, hydrolysis of a corresponding compound of formula II in which R^{9a}, R^{9e}, R^{9f} and R^{9j} do not represent H, or, for compounds of formula II in which X¹ represents —C(O)OR^{9a} or —P(O)(OR^{9f})₂, in which R^{9a} and R^{9f} represent H, a corresponding compound of formula II in which X¹ represents —C(O)N(H)S(O)₂R¹¹, —P(O)(OR^{9g})N(R^{10h})R^{9h} or —P(O)(N(R¹⁰ⁱ)R⁹ⁱ)₂ (as appropriate);
(z) for compounds of formula II in which X¹ represents —C(O)OR^{9a}—S(O)₃R^{9e}, —P(O)(OR^{9f})₂, —P(O)(OR^{9g})N(R^{10h})R^{9h} or —B(OR^{9j})₂ and R^{9a}, R^{9e}, R^{9f}, R^{9g} and R^{9j} (i.e. those R⁹ groups attached to an oxygen atom) do not represent H:
   (A) esterification of a corresponding compound of formula II in which R^{9a}, R^{9e}, R^{9f}, R^{9g} and R^{9j} represent H; or
   (B) trans-esterification of a corresponding compound of formula II in which R^{9a}, R^{9e}, R^{9f}, R^{9g} and R^{9j} do not represent H (and do not represent the same value of the corresponding R^{9a}, R^{9e}, R^{9f}, R^{9g} and R^{9j} group in the compound of formula II to be prepared),
   in the presence of the appropriate alcohol of formula XXIII as hereinbefore defined;
(aa) for compounds of formula II in which Q represents a C₁ alkylene group substituted with G¹, in which G¹ represents -A1-R^{12a}, A¹ represents —C(O)A²-, A² represents a single bond and R^{12a} represents H, and X² represents —C(O)OR^{9a}, in which R^{9a} is other than H, reaction of a corresponding compound of formula II in which the C₁ alkylene group that Q represents is unsubstituted with C₁₋₆ alkyl formate in the presence of a suitable base;
(ab) for compounds of formula II in which X¹ represents —C(O)N(R^{10b})R^{9b}, —C(O)N(H)C(═NR^{9c})N(R^{10d})R^{9d}, —C(O)N(H)CN or —C(O)N(H)S(O)₂R¹¹ reaction of a corresponding compound of formula II in which X¹ represents —C(O)OR^{9a} with a compound of formula XX as hereinbefore defined, for example under reaction conditions such as those described in respect of preparation of compounds of formula I (process (xix)) above;
(ac) for compounds of formula II in which X¹ represents -Q-X², Q represents a single bond and X² represents C₁₋₈ alkyl or heterocycloalkyl substituted ax to the indole ring by a G¹ substituent in which G¹ represents -A¹-R^{2a}, A¹ represents —OA⁵-, A⁵ represents a single bond and R^{12a} represents H, reaction of a corresponding compound of formula II in which X¹ represents H with a compound corresponding to a compound of formula VI, but in which X^{1b} represents -Q-X², Q represents a single bond and X² represents C₁₋₈ alkyl or heterocycloalkyl, both of which groups are substituted by a Z¹ group in which Z¹ represents ═O, for example under reaction conditions similar to those described hereinbefore in respect of preparation of compounds of formula I (process (xxxi)) above;
(ad) for compounds of formula II in which X¹ represents -Q-X², Q represents a single bond and X² represents C₂₋₈ alkyl substituted (e.g. α to the indole ring) by a G¹ substituent in which G¹ represents -A¹-R^{12a}, A¹ represents —OA⁵-, A⁵ represents a single bond and R^{12a} represents H, reaction of a corresponding compound of formula II in which X² represents C₁₋₇ alkyl substituted (e.g. α to the indole ring) by a Z group in which Z¹ represents ═O, with the corresponding Grignard reagent derivative of a compound of formula V in which L² represents chloro, bromo or iodo, X^{1a} represents -Q-X², Q is a single bond and X² represents C₁₋₇ alkyl, under conditions known to those skilled in the art;
(ae) for compounds of formula II in which X¹ represents -Q-X², Q represents a single bond, and X² represents C₁₋₈ alkyl or heterocycloalkyl, both of which are unsubstituted in the position a to the indole ring, reduction of a corresponding compound of formula II in which X² represents C₁₋₈ alkyl substituted α to the indole ring by a G¹ substituent in which G¹ represents -A¹-R^{2a}, A¹ represents —OA⁵-, A⁵ represents a single bond and R^{12a} represents H, for example under reaction conditions similar to those described hereinbefore in respect of preparation of compounds of formula I (process (xxxiii)) above;
(af) for compounds of formula II in which X¹ represents -Q-X², Q represents a single bond and X² represents C₁₋₈ alkyl or heterocycloalkyl, neither of which are substituted by Z¹ in which Z¹ represents ═O, reduction of a corresponding compound of formula II in which X² represents C₁₋₈ alkyl or heterocycloalkyl, which groups are substituted by one or more Z¹ groups in which Z¹ represents ═O, for example under reaction conditions similar to those described hereinbefore in respect of preparation of compounds of formula I (process (xxxiv)) above; or
(ag) for compounds of formula II in which X¹ represents —N(R^{9k})-J-R^{10k}, reaction of a compound of formula XXIX as hereinbefore defined, with a compound of formula VI in which X^{1b} represents —N(R^{9k})-J-R^{10k} and R^{9k}, R^{10k} and J are as hereinbefore defined, for example under conditions similar to those described hereinbefore in respect of preparation of compounds of formula I (process (xxxv)) above.

Compounds of formula IV may be prepared as follows:
(a) Reaction of a compound of formula XXIV as hereinbefore defined with a compound of formula XXXII,

R¹L² XXXII
wherein R¹ and L² are as hereinbefore defined or a compound of formula III as hereinbefore defined, for example under reaction conditions similar to those described hereinbefore in respect of preparation of compounds of formula I (processes (ii) and (i), respectively) above; or
(b) for compounds of formula IV wherein L¹ represents a sulfonate group, reaction of a compound of formula XVII as hereinbefore defined, with an appropriate reagent for the conversion of the hydroxyl group to the sulfonate group (e.g. tosyl chloride, mesyl chloride, triflic anhydride and the like) under conditions known to those skilled in the art.

Compounds of formula VII may be prepared by:
(a) For compounds of formula VII in which D represents a single bond, —C(O)—, —C(R⁷)(R⁸)—, C₂₋₄ alkylene or —S(O)₂—, reaction of a compound of formula XXXIII,

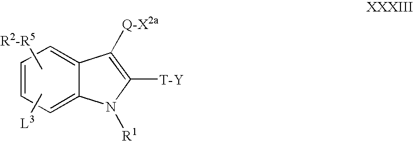

wherein Q, X^{2a}, L³, R¹, R²-R⁵, T and Y are as hereinbefore defined (L³ in particular may represent halo, such as bromo) with a compound of formula XI as hereinbefore defined (in which L⁴ may in particular represent —B(OH₂)), for example under reaction conditions similar to those described hereinbefore in respect of preparation of compounds of formula I (process (x)) above;
(b) reaction of a compound of formula XXV as hereinbefore defined with a compound of formula III as hereinbefore defined, for example under reaction conditions similar to those described hereinbefore in respect of preparation of compounds of formula I (process (i)) above); or
(c) for compounds of formula VII in which Q represents a single bond and X^{2a} represents —CHO, reaction of a corresponding compound of formula I in which X¹ represents H with a mixture of DMF and, for example, oxalyl chloride, phosgene or P(O)Cl₃ (or the like) in an appropriate solvent system (e.g. DMF or dichloromethane).

Compounds of formula X may be prepared by reaction of a compound of formula XXVI as hereinbefore defined, with a compound of formula III as hereinbefore defined, for example under reaction conditions similar to those described hereinbefore in respect of preparation of compounds of formula I (process (i)) above.

Compounds of formula X in which L³ represents L² may be prepared by reaction of a compound of formula X in which L³ represents L¹, with an appropriate reagent for the conversion of the L¹ group to the L² group. This conversion may be performed by methods known to those skilled in the art, for example, compounds of formula X, in which L³ is 4,4,5,5-tetramethyl-1,3,2-dioxaborolan-2-yl may be prepared by reaction of the reagent bis(pinacolato)diboron with a compound of formula X in which L³ represents L¹, for example under reaction conditions similar to those described hereinbefore in respect of preparation of compounds of formula I (process (ii)) above).

Compounds of formulae XV and XXVIII may be prepared by reaction of a corresponding compound of formula IV, or XXIV, respectively, with a compound of formula XXXIV,

R^{9a}NH₂ XXXIV

wherein R^{9a} is as hereinbefore defined, for example under reaction conditions similar to those described hereinbefore in respect of preparation of compounds of formula I (process (ii)) above).

Compounds of formula XIX may be prepared by standard techniques. For example:
(a) compounds of formula XIX in which R^{9a} is other than H may be prepared by reaction of a compound of formula XXXV,

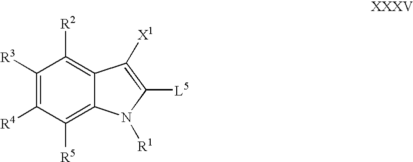

wherein L⁵, X¹, R¹, R², R³, R⁴ and R⁵ are as hereinbefore defined, with a compound of formula XXXVI,

L¹⁻T-C(O)OR^{9a1} XXXVI

wherein R^{9a1} represents R^{9a} provided that it does not represent H, and L¹ and T are as hereinbefore defined, for example under similar reaction conditions to those described hereinbefore in respect of process (xx) above, followed by (if necessary) deprotection under standard conditions;
(b) compounds of formula XIX in which T is a single bond, may alternatively be prepared by reaction of a corresponding compound of formula XXXV in which L⁵ is a sulfonate group (e.g. a triflate) or, preferably, a halo group (e.g. bromo or iodo) with CO (or a reagent that is a suitable source of CO (e.g. Mo(CO)₆ or Co₂(CO)₈)), in the presence of a compound of formula XXIIA as hereinbefore defined and an appropriate catalyst system (e.g. a palladium catalyst such as one described hereinbefore in respect of process step (ii)) under conditions known to those skilled in the art; and
(c) compounds of formula XIX in which T represents a single bond and R^{9a} represents H may be prepared by reaction of a compound of formula XXXV in which L⁵ represents either an alkali metal or —Mg-halide with carbon dioxide, followed by acidification.

Compounds of formula XXIV may be prepared by standard techniques. For example compounds of formula XXIV in which D represents a single bond, —C(O)—, —C(R⁷)(R⁸)—, C₂₋₄ alkylene or —S(O)₂, may be prepared by reaction of a compound of formula XXXVII,

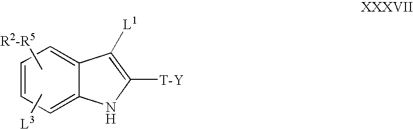

wherein L¹, L³, R²—R⁵ T and Y are as hereinbefore defined with a compound of formula XI as hereinbefore defined, for example under reaction conditions similar to those described hereinbefore in respect of preparation of compounds of formula I (process (x)) above.

Compounds of formulae XXV and XXXIII, in which Q represents a single bond and X^{2a} represents —CHO, may be prepared from compounds of formulae II, or X, respectively, in which X¹ represents H, by reaction with a mixture of DMF and, for example, oxalyl chloride, phosgene or P(O)Cl₃ (or the like) in an appropriate solvent system (e.g. DMF or dichloromethane) for example as described hereinbefore.

Compounds of formula XXX in which R^{9a} is other than H may be prepared by reaction of a compound of formula XXXVIII,

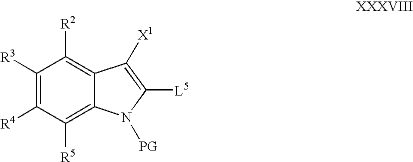

wherein PG represents a suitable protecting group, such as —S(O)₂Ph, —C(O)O—, —C(O)O/Bu or —C(O)N(Et)₂) and L⁵, X¹, R², R³, R⁴ and R⁵ are as hereinbefore defined, with a compound of formula XXXVIIIA,

L⁶T-C(O)OR^{9a1} XXXVIIIA

wherein L⁶, T and R^{9a1} are as hereinbefore defined, or a protected derivative thereof, for example under similar coupling conditions to those described hereinbefore, followed by deprotection of the resultant compound under standard conditions.

Compounds of formulae XXI, XXXI, XXXV and XXXVIII, in which L⁵ represents an appropriate alkali metal, such as lithium may be prepared by reaction of, in the case of a compound of formula XXI, or XXXI, a compound of formula XXXIX,

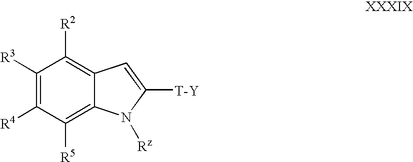

or, in the case of a compound of formula XXXV, or XXXVIII, a compound of formula XL,

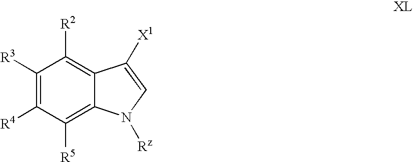

wherein, in both cases, R^{z} represents R¹ (in the case of compounds of formulae XXI and XXXV) or PG (in the case of compounds of formulae XXXI and XXXVIII), and PG, X¹, T, Y, R¹, R², R³, R⁴ and R⁵ are as hereinbefore defined, with an appropriate base, such as lithium diisopropylamide or BuLi under standard conditions. Compounds of formulae XXI, XXXI, XXXV and XXXVIII in which L⁵ represents another group (such as a zinc-based group or halo) may be prepared by an appropriate exchange reaction that will be well known to those skilled in the art. For example, compounds of formulae XXI, XXXI, XXXV and XXXVIII in which L⁵ represents —Mg-halide may be prepared from a corresponding compound of formula XXI, XXXI, XXXV or XXXVIII (as appropriate) in which L⁵ represents halo, for example under conditions such as those described hereinbefore in respect of process step (x). Compounds of formulae XXI, XXXI, XXXV and XXXVIII in which L⁵ represents, for example, a zinc-based group, halo or a boronic acid group may be prepared by reacting a corresponding compound of formula XXI, XXXI, XXXV or XXXVIII in which L⁵ represents an alkali metal with an appropriate reagent for introduction of the relevant group, for example by a metal exchange reaction (e.g. a Zn transmetallation), by reaction with a suitable reagent for the introduction of a halo group (for example, a reagent described hereinbefore in respect of preparation of compounds of formula I (process (xvi)), for the introduction of a boronic acid group, reaction with, for example, boronic acid or a protected derivative thereof (e.g. bis(pinacolato)diboron or triethyl borate). All of these reactions may be followed by (if necessary) deprotection under standard conditions.

Compounds of formulae III, V, VA, VI, VIII, IXA, IXB, IXC, XI, XII, XIII, XIV, XVI, XVII, XVIII, XX, XXII, XXIIA, XXIIB, XXIII, XXVI, XXVII, XXIX, XXXII, XXXIV, XXXVI, XXXVII, XXXVIIIA, XXXIX and XL are either commercially available, are known in the literature, or may be obtained either by analogy with the processes described herein, or by conventional synthetic procedures, in accordance with standard techniques, from available starting materials using appropriate reagents and reaction conditions. In this respect, the skilled person may refer to inter alia “Comprehensive Organic Synthesis” by B. M. Trost and I. Fleming, Pergamon Press, 1991.

Indoles of formulae II, IV, VII, X, XIII, XV, XVII, XIX, XXI, XXIV, XXV, XXVI, XXVII, XXVIII, XXIX, XXX, XXXI, XXXIII, XXXV, XXXVII, XXXVIII, XXXIX and XL may also be prepared with reference to a standard heterocyclic chemistry textbook (e.g. “Heterocyclic Chemistry” by J. A. Joule, K. Mills and G. F. Smith, 3^{rd} edition, published by Chapman & Hall or “Comprehensive Heterocyclic Chemistry II” by A. R. Katritzky, C. W. Rees and E. F. V. Scriven, Pergamon Press, 1996) and/or made according to the following general procedures.

For example, compounds of formulae II, XXVI, XXVII and XXX in which X¹ is as hereinbefore defined but not halo may be prepared by reaction of a compound of formula XLI,

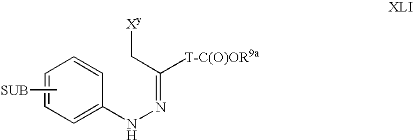

wherein SUB represents the substitution pattern that is present in the relevant compound to be formed (in this case, the compound of formula II, XXVI, XXVII or XXX, respectively), X^{y} represents X¹ but not halo, and R^{9a}, X¹ and T are as hereinbefore defined, under Fischer indole synthesis conditions known to the person skilled in the art, followed by, in the case of compounds of formulae II, XXVI and XXVII, conversion of the carboxylic acid or ester moiety to the appropriate amide using techniques such as those described hereinbefore.

Compounds of formulae II, XXVI, XXVII and XXX in which X¹ represents H may be prepared by reaction of a compound of formula XLII,

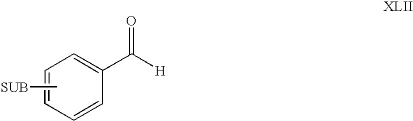

wherein SUB is as hereinbefore defined with a compound of formula XLIII,

N₃CH₂-T-Y XLIII

wherein T is as hereinbefore defined and preferably a single bond or optionally substituted arylene or heteroarylene, and Y is as hereinbefore defined and in the case of preparation of compounds of formula XXX, is —C(O)OR^{9a}, under conditions known to the person skilled in the art (i.e. conditions to induce a condensation reaction, followed by a thermally induced cyclisation), followed by, in the case of compounds of formulae II, XXVI and XXVII, conversion of the carboxylic acid or ester moiety to the appropriate amide using techniques such as those described hereinbefore.

Compounds of formulae XVII and XXIX may be prepared by reaction of a compound of formula XLIV,

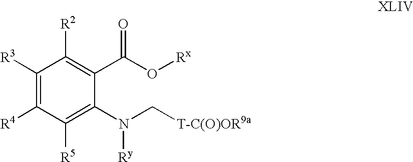

wherein R^{x} represents a C₁₋₆ alkyl group, R^{y} represents either R¹ (as required for the formation of compounds of formula XVII), hydrogen (as required for the formation of compounds of formula XXIX) or a nitrogen-protected derivative thereof, and R¹, R², R³, R⁴, R⁵, R^{9a} and T are as hereinbefore defined for example under cyclisation conditions known to those skilled in the art, followed by conversion of the carboxylic acid or ester moiety to the appropriate amide using techniques such as those described hereinbefore.

Compounds of formulae II and XXVI in which X¹ represents —NH₂ and T represents a single bond may be prepared by reaction of a compound of formula XLIVA,

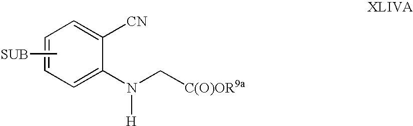

wherein SUB and R^{9a} are as hereinbefore defined, for example under intramolecular cyclisation conditions known to those skilled in the art, followed by conversion of the carboxylic acid or ester moiety to the appropriate amide using techniques such as those described hereinbefore.

Compounds of formulae II and XXVI in which X¹ represents H, —N(R^{9k})-J-R^{10k} or -Q-X² in which Q represents a single bond or —C(O)— may alternatively be prepared by reaction of a compound of formula XLV,

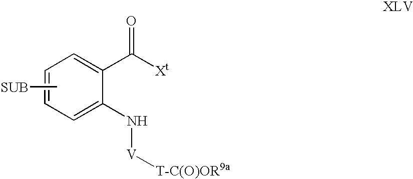

wherein V represents either —C(O)— or —CH₂—, X^{t} represents H, —N(R^{9k})-J-R^{10k} or -Q-X² in which Q represents a single bond or —C(O)— and SUB, R^{9a}, R^{9k}, R^{10k}, J, T and Y are as hereinbefore defined. When V represents —C(O)—, the intramolecular cyclisation may be induced by a reducing agent such as TiCl₃/C₈K, TiCl₄/Zn or SmI₂ under conditions known to the skilled person, for example, at room temperature in the presence of a polar aprotic solvent (such as THF). When V represents —CH₂—, the reaction may be performed in the presence of base under intramolecular condensation reaction conditions known to the skilled person, followed by conversion of the carboxylic acid or ester moiety to the appropriate amide using techniques such as those described hereinbefore.

Compounds of formula XXVII in which the -D^{c}H group is at the 5-position and D^{c} represents —O— (i.e. R³ is —OH), R², R⁴ and R⁵ all represent H, may be prepared by way of Nenitzescu indole synthesis by reaction of a compound of formula XLVI,

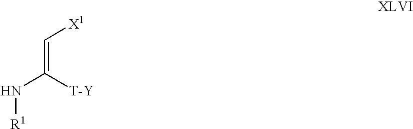

or a tautomer thereof, wherein X¹ is as hereinbefore defined and preferably —C(O)OR^{9a} and T, Y, R¹ and R^{9a} are as hereinbefore defined with benzoquinone under conditions that are known to those skilled in the art.

Compounds of formula XLI may be prepared by:
(a) reaction of a compound of formula XLVII,

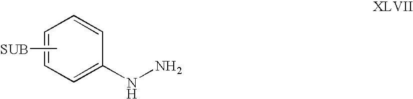

wherein SUB is as hereinbefore defined with a compound of formula XLVIII,

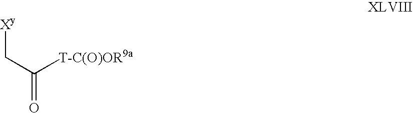

wherein X^{y}, T and R^{9a} are as hereinbefore defined under condensation conditions known to the skilled person;
(b) reaction of a compound of formula XLIX,

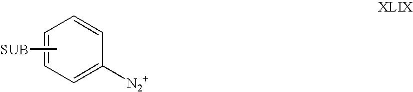

wherein SUB is as hereinbefore defined with a compound of formula L,

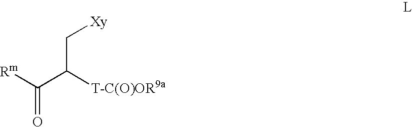

wherein R^{m} represents OH, O—C₁₋₆ alkyl or C₁₋₆ alkyl and X^{y}, T and R^{9a} are as hereinbefore defined, for example under Japp-Klingemann conditions known to the skilled person.

Compounds of formula XLV may be prepared by reaction of a compound of formula LI,

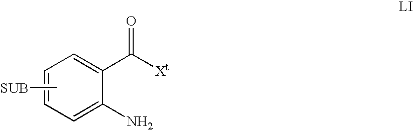

wherein SUB and X^{t} are as hereinbefore defined with a compound of formula LII,

R^{9a}OC(O)-T-V—Cl LII

wherein T, R^{9a} and V are as hereinbefore defined, under standard coupling conditions.

Compounds of formulae XLII, XLIII, XLIV, XLIVA, XLVI, XLVII, XLVIII, XLIX, L, LI and LII are either commercially available, are known in the literature, or may be obtained either by analogy with the processes described herein, or by conventional synthetic procedures, in accordance with standard techniques, from available starting materials using appropriate reagents and reaction conditions. In this respect, the skilled person may refer to inter alia “Comprehensive Organic Synthesis” by B. M. Trost and 1. Fleming, Pergamon Press, 1991.

The substituents X¹, R¹, R², R³, R⁴, R⁵ and Y in final compounds of the invention or relevant intermediates may be modified one or more times, after or during the processes described above by way of methods that are well known to those skilled in the art. Examples of such methods include substitutions, reductions, oxidations, alkylations, acylations, hydrolyses, esterifications, and etherifications. The precursor groups can be changed to a different such group, or to the groups defined in formula I, at any time during the reaction sequence. In this respect, the skilled person may also refer to “Comprehensive Organic Functional Group Transformations” by A. R. Katritzky, O. Meth-Cohn and C. W. Rees, Pergamon Press, 1995.

Compounds of the invention may be isolated from their reaction mixtures using conventional techniques.

It will be appreciated by those skilled in the art that, in the processes described above and hereinafter, the functional groups of intermediate compounds may need to be protected by protecting groups.

The protection and deprotection of functional groups may take place before or after a reaction in the above-mentioned schemes.

Protecting groups may be removed in accordance with techniques that are well known to those skilled in the art and as described hereinafter. For example, protected compounds/intermediates described herein may be converted chemically to unprotected compounds using standard deprotection techniques.

The type of chemistry involved will dictate the need, and type, of protecting groups as well as the sequence for accomplishing the synthesis.

The use of protecting groups is fully described in “Protective Groups in Organic Chemistry”, edited by J W F McOmie, Plenum Press (1973), and “Protective Groups in Organic Synthesis”, 3^{rd} edition, T. W. Greene & P. G. M. Wutz, Wiley-Interscience (1999).

### Medical and Pharmaceutical Uses

Compounds of the invention are indicated as pharmaceuticals. According to a further aspect of the invention there is provided a compound of the invention, as hereinbefore defined but without the proviso, for use as a pharmaceutical.

Although compounds of the invention may possess pharmacological activity as such, certain pharmaceutically-acceptable (e.g. “protected”) derivatives of compounds of the invention may exist or be prepared which may not possess such activity, but may be administered parenterally or orally and thereafter be metabolised in the body to form compounds of the invention. Such compounds (which may possess some pharmacological activity, provided that such activity is appreciably lower than that of the “active” compounds to which they are metabolised) may therefore be described as “prodrugs” of compounds of the invention.

By “prodrug of a compound of the invention”, we include compounds that form a compound of the invention, in an experimentally-detectable amount, within a predetermined time (e.g. about 1 hour), following oral or parenteral administration. All prodrugs of the compounds of the invention are included within the scope of the invention.

Furthermore, certain compounds of the invention may possess no or minimal pharmacological activity as such, but may be administered parenterally or orally, and thereafter be metabolised in the body to form compounds of the invention that possess pharmacological activity as such. Such compounds (which also includes compounds that may possess some pharmacological activity, but that activity is appreciably lower than that of the “active” compounds of the invention to which they are metabolised), may also be described as “prodrugs”.

Thus, the compounds of the invention are useful because they possess pharmacological activity, and/or are metabolised in the body following oral or parenteral administration to form compounds which possess pharmacological activity.

Compounds of the invention are particularly useful because they may inhibit the activity of a member of the MAPEG family.

Compounds of the invention are particularly useful because they may inhibit (for example selectively) the activity of prostaglandin E synthases (and particularly microsomal prostaglandin E synthase-1 (mPGES-1)), i.e. they prevent the action of mPGES-1 or a complex of which the mPGES-1 enzyme forms a part, and/or may elicit a mPGES-1 modulating effect, for example as may be demonstrated in the test described below. Compounds of the invention may thus be useful in the treatment of those conditions in which inhibition of a PGES, and particularly mPGES-1, is required.

Compounds of the invention may inhibit the activity of leukotriene C₄ (LTC₄), for example as may be shown in a test such as that described in Eur. J. Biochem., 208, 725-734 (1992), and may thus be useful in the treatment of those conditions in which inhibition of LTC₄ is required. Compounds of the invention may also inhibit the activity of 5-lipoxygenase-activating protein (FLAP), for example as may be shown in a test such as that described in Mol. Pharmacol., 41, 873-879 (1992).

Compounds of the invention are thus expected to be useful in the treatment of inflammation.

The term “inflammation” will be understood by those skilled in the art to include any condition characterised by a localised or a systemic protective response, which may be elicited by physical trauma, infection, chronic diseases, such as those mentioned hereinbefore, and/or chemical and/or physiological reactions to external stimuli (e.g. as part of an allergic response). Any such response, which may serve to destroy, dilute or sequester both the injurious agent and the injured tissue, may be manifest by, for example, heat, swelling, pain, redness, dilation of blood vessels and/or increased blood flow, invasion of the affected area by white blood cells, loss of function and/or any other symptoms known to be associated with inflammatory conditions.

The term “inflammation” will thus also be understood to include any inflammatory disease, disorder or condition per se, any condition that has an inflammatory component associated with it, and/or any condition characterised by inflammation as a symptom, including inter alia acute, chronic, ulcerative, specific, allergic and necrotic inflammation, and other forms of inflammation known to those skilled in the art. The term thus also includes, for the purposes of this invention, inflammatory pain, pain generally and/or fever.

Accordingly, compounds of the invention may be useful in the treatment of asthma, chronic obstructive pulmonary disease, pulmonary fibrosis, inflammatory bowel disease, irritable bowel syndrome, inflammatory pain, fever, migraine, headache, low back pain, fibromyalgia, myofascial disorders, viral infections (e.g. influenza, common cold, herpes zoster, hepatitis C and AIDS), bacterial infections, fungal infections, dysmenorrhea, burns, surgical or dental procedures, malignancies (e.g. breast cancer, colon cancer, and prostate cancer), hyperprostaglandin E syndrome, classic Bartter syndrome, atherosclerosis, gout, arthritis, osteoarthritis, juvenile arthritis, rheumatoid arthritis, rheumatic fever, ankylosing spondylitis, Hodgkin's disease, systemic lupus erythematosus, vasculitis, pancreatitis, nephritis, bursitis, conjunctivitis, iritis, scleritis, uveitis, wound healing, dermatitis, eczema, psoriasis, stroke, diabetes mellitus, neurodegenerative disorders such as Alzheimer's disease and multiple sclerosis, autoimmune diseases, allergic disorders, rhinitis, ulcers, coronary heart disease, sarcoidosis and any other disease with an inflammatory component.

Compounds of the invention may also have effects that are not linked to inflammatory mechanisms, such as in the reduction of bone loss in a subject. Conditions that may be mentioned in this regard include osteoporosis, osteoarthritis, Paget's disease and/or periodontal diseases. Compounds the invention may thus also be useful in increasing bone mineral density, as well as the reduction in incidence and/or healing of fractures, in subjects.

Compounds of the invention are indicated both in the therapeutic and/or prophylactic treatment of the above-mentioned conditions.

According to a further aspect of the present invention, there is provided a method of treatment of a disease which is associated with, and/or which can be modulated by inhibition of, a member of the MAPEG family such as a PGES (such as mPGES-1), LTC₄ and/or FLAP and/or a method of treatment of a disease in which inhibition of the activity of a member of the MAPEG family such as a PGES (and particularly mPGES-1), LTC₄ and/or FLAP is desired and/or required (e.g. inflammation), which method comprises administration of a therapeutically effective amount of a compound of the invention, as hereinbefore defined but without the proviso, to a patient suffering from, or susceptible to, such a condition.

“Patients” include mammalian (including human) patients.

The term “effective amount” refers to an amount of a compound, which confers a therapeutic effect on the treated patient. The effect may be objective (i.e. measurable by some test or marker) or subjective (i.e. the subject gives an indication of or feels an effect).

Compounds of the invention will normally be administered orally, intravenously, subcutaneously, buccally, rectally, dermally, nasally, tracheally, bronchially, sublingually, by any other parenteral route or via inhalation, in a pharmaceutically acceptable dosage form.

Compounds of the invention may be administered alone, but are preferably administered by way of known pharmaceutical formulations, including tablets, capsules or elixirs for oral administration, suppositories for rectal administration, sterile solutions or suspensions for parenteral or intramuscular administration, and the like.

Such formulations may be prepared in accordance with standard and/or accepted pharmaceutical practice.

According to a further aspect of the invention there is thus provided a pharmaceutical formulation including a compound of the invention, as hereinbefore defined but without the proviso, in admixture with a pharmaceutically acceptable adjuvant, diluent or carrier.

Compounds of the invention may also be combined with other therapeutic agents that are useful in the treatment of inflammation (e.g. NSAIDs and coxibs).

According to a further aspect of the invention, there is provided a combination product comprising:
(A) a compound of the invention, as hereinbefore defined but without the proviso; and
(B) another therapeutic agent that is useful in the treatment of inflammation,
   wherein each of components (A) and (B) is formulated in admixture with a pharmaceutically-acceptable adjuvant, diluent or carrier.

Such combination products provide for the administration of a compound of the invention in conjunction with the other therapeutic agent, and may thus be presented either as separate formulations, wherein at least one of those formulations comprises a compound of the invention, and at least one comprises the other therapeutic agent, or may be presented (i.e. formulated) as a combined preparation (i.e. presented as a single formulation including a compound of the invention and the other therapeutic agent).

Thus, there is further provided:

(1) a pharmaceutical formulation including a compound of the invention, as hereinbefore defined but without the proviso, another therapeutic agent that is useful in the treatment of inflammation, and a pharmaceutically-acceptable adjuvant, diluent or carrier; and
(2) a kit of parts comprising components:
(a) a pharmaceutical formulation including a compound of the invention, as hereinbefore defined but without the proviso, in admixture with a pharmaceutically-acceptable adjuvant, diluent or carrier; and
(b) a pharmaceutical formulation including another therapeutic agent that is useful in the treatment of inflammation in admixture with a pharmaceutically-acceptable adjuvant, diluent or carrier,
   which components (a) and (b) are each provided in a form that is suitable for administration in conjunction with the other.

Compounds of the invention may be administered at varying doses. Oral, pulmonary and topical dosages may range from between about 0.01 mg/kg of body weight per day (mg/kg/day) to about 100 mg/kg/day, preferably about 0.01 to about 10 mg/kg/day, and more preferably about 0.1 to about 5.0 mg/kg/day. For e.g. oral administration, the compositions typically contain between about 0.01 mg to about 500 mg, and preferably between about 1 mg to about 100 mg, of the active ingredient. Intravenously, the most preferred doses will range from about 0.001 to about 10 mg/kg/hour during constant rate infusion. Advantageously, compounds may be administered in a single daily dose, or the total daily dosage may be administered in divided doses of two, three or four times daily.

In any event, the physician, or the skilled person, will be able to determine the actual dosage which will be most suitable for an individual patient, which is likely to vary with the route of administration, the type and severity of the condition that is to be treated, as well as the species, age, weight, sex, renal function, hepatic function and response of the particular patient to be treated. The above-mentioned dosages are exemplary of the average case; there can, of course, be individual instances where higher or lower dosage ranges are merited, and such are within the scope of this invention.

Compounds of the invention may have the advantage that they are effective, and preferably selective, inhibitors of a member of MAPEG family, e.g. inhibitors of prostaglandin E synthases (PGES) and particularly microsomal prostaglandin E synthase-1 (mPGES-1). Compounds of the invention may reduce the formation of the specific arachidonic acid metabolite PGE₂ without reducing the formation of other COX generated arachidonic acid metabolites, and thus may not give rise to the associated side-effects mentioned hereinbefore.

Compounds of the invention may also have the advantage that they may be more efficacious than, be less toxic than, be longer acting than, be more potent than, produce fewer side effects than, be more easily absorbed than, and/or have a better pharmacokinetic profile (e.g. higher oral bioavailability and/or lower clearance) than, and/or have other useful pharmacological, physical, or chemical properties over, compounds known in the prior art, whether for use in the above-stated indications or otherwise.

### Biological Test

In the assay mPGES-1 catalyses the reaction where the substrate PGH₂ is converted to PGE₂. mPGES-1 is expressed in E. coli and the membrane fraction is dissolved in 20 mM NaPi-buffer pH 8.0 and stored at −80° C. In the assay mPGES-1 is dissolved in 0.1 M KPi-buffer pH 7.35 with 2.5 mM glutathione. The stop solution consists of H₂O/MeCN (7/3), containing FeCl₂ (25 mM) and HCl (0.15 M). The assay is performed at room temperature in 96-well plates. Analysis of the amount of PGE₂ is performed with reversed phase HPLC (Waters 2795 equipped with a 3.9×150 mm C18 column). The mobile phase consists of H₂O/MeCN (7/3), containing TFA (0.056%), and absorbance is measured at 195 nm with a Waters 2487 UV-detector.

The following is added chronologically to each well:
1. 100 μL mPGES-1 in KPi-buffer with glutathione. Total protein concentration: 0.02 mg/mL.
2. 1 μL inhibitor in DMSO. Incubation of the plate at room temperature for 25 minutes.
3. 4 μL of a 0.25 mM PGH₂ solution. Incubation of the plate at room temperature for 60 seconds.
4. 100 μL stop solution.
   180 μL per sample is analyzed with HPLC.

### EXAMPLES

The invention is illustrated by way of the following examples, in which the following abbreviations may be employed:
DMAP 4,4-dimethylaminopyridine
DMF dimethylformamide
DMSO dimethylsulfoxide
EtOAc ethyl acetateHBTU O-(1H-benzotriazol-1-yl)-1,1,3,3-tetramethyluronium hexafluorophosphate
MeCN acetonitrile
MS mass spectrum
NMR nuclear magnetic resonance
rt room temperature
TFA trifluoroacetic acid
THF tetrahydrofuran

Starting materials and chemical reagents specified in the syntheses described below are commercially available from, e.g. Sigma-Aldrich Fine Chemicals.

### Example 1

### N-[5-(4-tert-Butylphenyl)-1-(4-cyclopentyloxyphenyl)indole-2-carbonyl]-methanesulfonamide

### (a) 5-(4-tert-Butylphenyl)indole-2-carboxylic acid ethyl ester

A mixture of 5-bromoindole-2-carboxylic acid ethyl ester (3.48 g, 13 mmol), 4-tert-butylphenylboronic acid (4.63 g, 26 mmol), K₃PO₄ (9.93 g, 45 mmol), Pd(OAc)₂ (146 mg, 0.65 mmol), tri-o-tolylphosphine (396 mg, 1.3 mmol), EtOH (20 mL) and toluene (10 mL) was stirred under argon for 20 min at rt, and heated at 100° C. for 24 h. The mixture was allowed to cool to rt, poured into NaHCO₃ (aq, sat) and extracted with EtOAc. The combined extracts were washed with water and brine, dried (Na₂SO₄), concentrated and purified by chromatography to give sub-title compound (3.27 g, 78%).

### (b) 5-(4-tert-Butylphenyl)-1-(4-cyclopentyloxyphenyl)indole-2-carboxylic acid ethyl ester

### Method A

5-(4-tert-Butylphenyl)indole-2-carboxylic acid ethyl ester (0.95 g, 2.96 mmol; see step (a) above), CuI (56 mg, 0.30 mmol), K₃PO₄ (1.25 g, 5.90 mmol), N,N′-dimethyl-1,2-diaminoethane (91 μL, 0.89 mmol) and 1-bromo-4-cyclopentyl-oxybenzene (1.42 g, 5.9 mmol) in toluene (10 mL) was heated at 110° C. for 24 h. The mixture was diluted with EtOAc and washed with NaHCO₃ (aq, sat), HCl (aq, 0.1 M) and brine, dried (Na₂SO₄), concentrated and purified by chromatography to give the sub-title compound (1.96 g, 69%).

### Method B

Anhydrous CH₂Cl₂ (80 mL), followed by Et₃N (3.10 mL, 22.0 mmol) and pyridine (1.80 mL, 22.0 mmol) were added to 5-(4-tert-butylphenyl)indole-2-carboxylic acid ethyl ester (3.54 g, 11.0 mmol; see step (a) above), Cu(OAc)₂ (4.00 g, 22.0 mmol), 3 Å molecular sieves (ca. 7 g) and 4-cyclopentyloxy-phenylboronic acid (4.54 g, 22.0 mmol). The mixture was stirred vigorously at rt for 48 h, and additional Et₃N (1.6 mL, 11.0 mmol), pyridine (0.90 mL, 11.0 mmol), Cu(OAc)₂ (2.00 g, 11.0 mmol) and 4-cyclopentyloxyphenylboronic acid (2.27 g, 11.0 mmol) were added and the mixture was stirred at rt for another 48 h. The mixture was filtered through Celite® and the solids washed with EtOAc. The filtrates were concentrated and purified by chromatography to afford the sub-title compound (3.7 g, 70%).

### (c) 5-(4-tert-Butylphenyl)-1-(4-cyclopentyloxyphenyl)indole-2-carboxylic acid

A mixture of 5-(4-tert-butylphenyl)-1-(4-cyclopentyloxyphenyl)indole-2-carboxylic acid ethyl ester (1.96 g, 4.07 mmol; see step (b)), NaOH (1.63 g, 40.7 mmol), water (5.0 mL) and EtOH (25 mL) was heated at reflux for 0.5 h. After cooling, the mixture was acidified with HCl (aq, 1 M) to pH 2 and extracted with EtOAc. The combined extracts were washed with water and brine, dried (Na₂SO₄) and concentrated to give the sub-title compound (1.84 g, 100%) which was employed in the next step without further purification.

### (d) 5-(4-tert-Butylphenyl)-1-(4-cyclopentyloxyphenyl)indole-2-carbonyl chloride

### Method A

SOCl₂ (48 μL, 0.66 mmol) and a few drops of DMF were added to a solution of 5-(4-tert-butylphenyl)-1-(4-cyclopentyloxyphenyl)indole-2-carboxylic acid (150 mg, 0.33 mmol, see step (c)) in Et₂O (14 mL). After 3.5 h at rt, the mixture was concentrated and the residue used in the next step without further purification.

### Method B

Oxalyl chloride (260 μL, 3.0 mmol) and a few drops of DMF were added to a solution of 5-(4-tert-butylphenyl)-1-(4-cyclopentyloxyphenyl)indole-2-carboxylic acid (450 mg, 1.0 mmol, see step (c)) in CH₂Cl₂ (20 mL). After 1 h at reflux, the mixture was concentrated and the residue used in the next step without further purification.

### (e) N-[5-(4-tert-Butylphenyl)-1-(4-cyclopentyloxyphenyl)indole-2-carbonyl]methanesulfonamide

A mixture of 5-(4-tert-butylphenyl)-1-(4-cyclopentyloxyphenyl)indole-2-carbonyl chloride (125 mg, 0.26 mmol, see step (d) above), methanesulfonamide (76 mg, 0.79 mmol), DMAP (32 mg, 0.26 mmol) and pyridine (2 mL) was heated at 80° C. for 12 h. The mixture was concentrated and the residue dissolved in EtOAc. The solution was washed with HCl (aq, 0.05 M), water and brine, dried (Na₂SO₄), concentrated and purified by chromatography to give the title compound (87 mg, 62%).

200 MHz ¹H-NMR (DMSO-d₆, ppm) δ 12.2 (1H, br s) 7.99 (1H, d, J=1.3 Hz) 7.67 (1H, s) 7.64-7.53 (3H, m) 7.51-7.41 (2H, m) 7.26-7.16 (21H, m) 7.08 (1H, d, J=8.8 Hz) 7.06-6.97 (2H, m) 4.93-4.81 (1H, m) 3.26 (3H, s) 2.04-1.50 (8H, m) 1.30 (9H, m)

### Example 2

### 2-{[5-(4-tert-Butylphenyl)-1-(4-cyclopentyloxyphenyl)indole-2-carbonyl]-amino}ethanesulfonic acid

A mixture of 5-(4-tert-butylphenyl)-1-(4-cyclopentyloxyphenyl)indole-2-carbonyl chloride (155 mg, 0.33 mmol, see step (d) Example 1), 2-amino-ethanesulfonic acid (45 mg, 0.36 mmol) and pyridine (4 mL) was stirred at rt for 20 h. The mixture was concentrated and the residue dissolved in dioxane and NaOH (aq, 0.5 M) and acidified to pH 1 with HCl (aq, 1 M). The dioxane was partially concentrated and the formed precipitate was collected and purified by chromatography to give the title compound (69 mg, 37%).

200 MHz ¹H-NMR (DMSO-d₆, ppm) δ 8.52-8.40 (1H, m) 7.95-7.89 (1H, m) 7.64-7.53 (2H, m) 7.52-7.40 (3H, m) 7.27-7.16 (2H, m) 7.15-7.05 (2H, m) 7.04-6.94 (2H, m) 4.92-4.80 (1H, m) 3.48-3.33 (2H, m) 2.62 (2H, t, J=6.9 Hz) 2.08-1.49 (8H, m) 1.30 (9H, m).

### Example 3

### N-[5-(4-tert-Butylphenyl)-1-(4-cyclopentyloxyphenyl)indole-2-carbonyl]-guanidine

A mixture of 5-(4-tert-butylphenyl)-1-(4-cyclopentyloxyphenyl)indole-2-carboxylic acid ethyl ester (96 mg, 0.2 mmol; see step (b) Example 1), guanine hydrochloride (191 mg, 2.0 mmol), sodium methoxide (108 mg, 0.2 mmol) and DMF (2.5 mL) was stirred at rt for 24 h. The mixture was diluted with EtOAc and washed several times with water and brine, dried (Na₂SO₄), concentrated and crystallised from Et₂O to give the title compound (25 mg, 25%).

200 MHz ¹H-NMR (DMSO-d₆, ppm) δ 7.90-7.85 (1H, m) 7.61-7.52 (2H, m) 7.50-7.38 (3H, m) 7.24-7.13 (3H, m) 7.04-6.93 (3H, m) 4.91-4.80 (1H, m) 2.10-1.50 (8H, m) 1.30 (9H, s)

### Example 4

### {[5-(4-tert-Butylphenyl)-1-(4-cyclopentyloxyphenyl)indole-2-carbonyl]-amino}acetic acid

### (a) {[5-(4-tert-Butylphenyl)-1-(4-cyclopentyloxyphenyl)indole-2-carbonyl]-amino}acetic acid ethyl ester

A solution of 5-(4-tert-butylphenyl)-1-(4-cyclopentyloxyphenyl)indole-2-carbonyl chloride (472 mg, 1.0 mmol, see step (d) Example I) in CH₂Cl₂ (10 mL) was added to glycine ethyl ester hydrochloride (209 mg, 1.5 mmol) and Et₃N (420 μL, 3.0 mmol) in CH₂Cl₂ (30 mL) at 0° C. After 12 h the mixture was poured into NaHCO₃ (aq, sat). The phases were separated and the organic layer was washed with NaHCO₃ (aq, sat), water and brine, dried (Na₂SO₄), concentrated and crystallised from petroleum ether/EtOAc to afford the sub-title compound (362 mg, 67%).

### (b) {[5-(4-tert-Butylphenyl)-1-(4-cyclopentyloxyphenyl)indole-2-carbonyl]-amino}acetic acid

A mixture of {[5-(4-tert-butylphenyl)-1-(4-cyclopentyloxyphenyl)indole-2-carbonyl]amino}acetic acid ethyl ester (100 mg, 0.19 mmol; see step (a) Example 4), dioxane (2 mL) and NaOH (aq, 2M, 1.0 mL, 2.0 mmol) was heated at 60° C. for 10 min. After cooling, a few drops of water was added, followed by HCl (aq, 2 M) until the pH was ca 2. The white precipitate was filtered off and dried to give the title compound (88 mg, 91%).

200 MHz ¹H-NMR (DMSO-d₆, ppm) δ 13.0-12.0 (1H, br s) 8.82 (1H, t, J=5.8 Hz) 7.95-7.94 (1H, m) 7.62-7.56 (2H, m) 7.52-7.42 (3H, m) 7.27 (1H, s) 7.22-7.16 (2H, m) 7.09 (1H, d, J=8.8 Hz) 7.01-6.93 (2H, m) 4.88-4.81 (1H, m) 3.81 (2H, d, J=5.8 Hz) 2.00-1.85 (2H, m) 1.81-1.51 (6H, m) 1.30 (9H, s)

### Example 5

### {[5-(4-tert-Butylphenyl)-1-(4-cyclopentyloxyphenyl)indole-2-carbonyl]-methylamino}acetic acid

The title compound was prepared in accordance with Example 4 from 5-(4-tert-butylphenyl)-1-(4-cyclopentyloxyphenyl)indole-2-carbonyl chloride and N-methyl glycine ethyl ester hydrochloride, followed by hydrolysis.

200 MHz ¹H-NMR (DMSO-d₆, ppm) δ 13.1-12.6 (1H, s) 7.93-7.91 (1H, m) 7.64-7.58 (2H, m) 7.55-7.45 (3H, m) 7.36-7.21 (3H, m) 7.04-6.99 (2H, m) 6.93 (0.6H, s, major amide rotamer) (0.4H, s, minor amide rotamer) 4.90-4.83 (1H, m) 4.27 (0.9H, s, minor rotamer) 4.05 (1:1H, s, major rotamer) 3.11 (1.8H, s, major amide rotamer) 2.91 (1.2H, s, minor amide rotamer) 1.99-1.60 (8H, m) 1.32 (9H, s)

### Example 6

### 2-Benzyloxycarbonylamino-3-{[5-(4-tert-butylphenyl)-1-(4-cyclopentyloxyphenyl)indole-2-carbonyl]amino}propionic acid

The title compound was prepared in accordance with Example 4 from 5-(4-tert-butylphenyl)-1-(4-cyclopentyloxyphenyl)indole-2-carbonyl chloride and N^{α}-carbobenzyloxy-2,3-diaminopropionic acid methyl ester hydrochloride, followed by hydrolysis.

200 MHz ¹H-NMR (DMSO-d₆, ppm) δ 12.8-12.6 (1H, br s) 8.59 (1H, t, J=5.2 Hz) 7.93-7.92 (1H, m) 7.61-7.43 (6H, m) 7.35-7.10 (9H, m) 6.99-6.93 (2H, m) 5.03 (2H, s) 4.87-4.79 (1H, m) 4.24-4.14 (1H, m) 3.57-3.45 (2H, m) 2.00-1.55 (8H, m) 1.30 (91H, s).

### Example 7

### 3-{[5-(4-tert-Butylphenyl)-1-(4-cyclopentyloxyphenyl)indole-2-carbonyl]-amino}propionic acid

### (a) 3-{[5-(4-tert-Butylphenyl)-1-(4-cyclopentyloxyphenyl)indole-2-carbonyl]-amino}propionic acid tert-butyl ester

The sub-title compound was prepared in accordance with Example 4 from 5-(4-tert-butylphenyl)-1-(4-cyclopentyloxyphenyl)indole-2-carbonyl chloride and β-alanine tert-butyl ester hydrochloride (55% yield) and was used in the next step without further purification.

### (b) 3-{[5-(4-tert-Butylphenyl)-1-(4-cyclopentyloxyphenyl)indole-2-carbonyl]-amino}propionic acid

TFA (1.0 mL, 13.5 mmol) was added to a solution of 3-{[5-(4-tert-butylphenyl)-1-(4-cyclopentyloxyphenyl)indole-2-carbonyl]amino}propionic acid tert-butyl ester (100 mg, 0.17 mmol; see step (a) Example 7) in CH₂Cl₂ (2 mL). The mixture was stirred at rt for 4 h and concentrated to afford the title compound as a white foam (80 mg, 90%).

200 MHz ¹H-NMR (DMSO-d₆, ppm) δ 13-11 (1H, br s) 8.54 (1H, t, J=5.4 Hz) 7.92-7.91 (1H, m) 7.61-7.55 (2H, m) 7.51-7.42 (3H, m) 7.22-7.15 (3H, m) 7.09 (1H, d, J=8.8 Hz) 7.02-6.95 (2H, m) 4.90-4.82 (1H, m) 3.38-3.29 (2H, m) 2.46-2.39 (2H, m) 2.01-1.55 (8H, m) 1.30 (9H, s).

### Example 8

### {[5-(3-Chlorophenoxy)-1-(4-isopropoxyphenyl)indole-2-carbonyl]amino}-acetic acid

### (a) 5-Benzyloxy-1-(4-isopropoxyphenyl)indole-2-carboxylic acid ethyl ester

A solution of 4-isopropoxyphenylbromide (150 mg, 0.7 mmol) in toluene (1.0 mL), followed by a solution of CuI (22.9 mg, 0.12 mmol) and N,N′-dimethyl-1,2-diaminoethane (25.5 μL, 0.24 mmol) in toluene (1.2 mL) were added to a mixture of K₃PO₄ (220 mg, 1.05 mmol) and 5-benzyloxyindole-2-carboxylic acid ethyl ester (150 mg, 0.5 mmol). The mixture was heated at 110° C. for 20 h, cooled and filtered. The precipitate was washed with acetone and the combined filtrates were concentrated and purified by chromatography to afford the sub-title compound (163 mg, 75%).

### (b) 5-Hydroxy-1-(4-isopropoxyphenyl)indole-2-carboxylic acid ethyl ester

A mixture of 5-benzyloxy-1-(4-isopropoxyphenyl)indole-2-carboxylic acid ethyl ester (1.00 g, 2.3 mmol; see step (a) above), HCl (aq, conc, 0.42 mL) and EtOAc (15 mL) was hydrogenated at ambient temperature and pressure over Pd—C (10%, 0.45 g, 0.043 mmol) for 1.5 h. The mixture was filtered and the filtrate concentrated and purified by chromatography to give the sub-title compound (0.70 g, 88%).

### (c) 5-(3-Chlorophenoxy)-1-(4-isopropoxyphenyl)indole-2-carboxylic acid ethyl ester

Anhydrous CH₂Cl₂ (15 mL), Et₃N (0.40 mL, 2.94 mmol) and pyridine (0.23 g, 2.94 mmol) were added to 5-hydroxy-1-(4-isopropoxyphenyl)indole-2-carboxylic acid ethyl ester (0.50 g, 1.47 mmol; see step (b) above), Cu(OAc)₂ (0.27 g, 1.47 mmol) and 3-chlorophenylboronic acid (0.46 g, 2.94 mmol). The mixture was stirred vigorously at rt for 72 h, filtered through Celite®, concentrated and purified by chromatography to afford the sub-title compound (0.32 g, 55%).

### (d) 5-(3-Chlorophenoxy)-1-(4-isopropoxyphenyl)indole-2-carboxylic acid

NaOH (266 mg, 6.6 mmol) in water (10 mL) was added to 5-(3-chlorophenoxy)-1-(4-isopropoxyphenyl)indole-2-carboxylic acid ethyl ester (300 mg, 0.66 mmol; see step (c) above) and acetone (20 mL). The mixture was stirred at rt for 24 h and concentrated to give the crude sub-title product (260 mg, 93%) which was used in the next step without further purification.

### (e) {[5-(3-Chlorophenoxy)-1-(4-isopropoxyphenyl)indole-2-carbonyl]amino}-acetic acid methyl ester

Pentafluorophenol (65 mg, 0.35 mmol) followed by dicyclohexylcarbodiimide (73 mg, 0.35 mmol) were added to 5-(3-chlorophenoxy)-1-(4-isopropoxyphenyl)-indole-2-carboxylic acid (150 mg, 0.35 mmol; see step (d) above) in EtOAc (25 mL) at 0° C. The mixture was stirred vigorously at 0° C. for 1 h and at rt for 3 h. Glycine methyl ester hydrochloride (40 mg, 0.35 mmol) and Et₃N (0.10 mL, 0.70 mmol) were added and the stirring was continued for 24 h. Filtration, concentration and purification by chromatography gave the sub-title product (150 mg, 86%).

### (f) {[5-(3-Chlorophenoxy)-1-(4-isopropoxyphenyl)indole-2-carbonyl]amino}-acetic acid

NaOH (120 mg, 3.00 mmol) in water (10 mL) was added to a solution of {[5-(3-chlorophenoxy)-1-(4-isopropoxyphenyl)indole-2-carbonyl]amino}acetic acid methyl ester (150 mg, 0.30 mmol; see step (e) above) in EtOH (10 mL). The mixture was stirred at rt for 20 h, neutralised with HCl (aq, conc), concentrated and purified by chromatography to afford the title compound (105 mg, 72%).

200 MHz ¹H-NMR (CDCl₃, ppm) δ 7.34-7.23 (3H, m, overlapped with CHCl₃) 7.18 (2H, d, J=8.4 Hz) 7.09 (1H, d, J=9.0 Hz) 7.04-6.94 (4H, m) 6.94-6.89 (1H, m) 6.85 (1H, dd, J=8.2, 2.0 Hz) 6.42 (1H, t, J=4.4 Hz) 6.6-6.0 (1H, br s) 4.60 (1H, septet, J=6.0 Hz) 4.12 (2H, d, J=4.4 Hz) 1.37 (6H, d, J=6.0 Hz).

### Example 9

### 5-(4-tert-Butylphenyl)-1-(4-cyclopentyloxyphenyl)indole-2-carboxylic acid (5-methylisoxazol-3-yl)amide

A mixture of 5-(4-tert-butylphenyl)-1-(4-cyclopentyloxyphenyl)indole-2-carbonyl chloride (189 mg, 0.4 mmol, see Example 1, step (d)), 3-amino-5-methylisoxazole (67 mg, 0.8 mmol) and pyridine (5 mL) was stirred at rt for 16 h. The mixture was concentrated and the residue dissolved in EtOAc. The solution was washed with HCl (aq, 0.05 M.), water and brine, dried (Na₂SO₄), concentrated and crystallised from Et₂O to give the title compound (114 mg, 55%).

200 MHz H-NMR (DMSO-d₆, ppm) δ 11.42-11.38 (1H, br s) 7.97 (1H, d, J=1.4 Hz) 7.65-7.50 (4H, m) 7.49-7.40 (2H, m) 7.27-7.18 (2H, m) 7.10 (1H, d, J=8.8 Hz) 7.04-6.94 (2H, m) 6.58-6.56 (1H, m) 4.91-4.80 (1H, m) 2.36-2.30 (3H, m) 2.00-1.51 (8H, m) 1.29 (9H, m).

### Example 10

### 5-(4-tert-Butylphenyl)-1-(4-cyclopentyloxyphenyl)indole-2-carboxylic acid (tetrazol-5-yl)amide

The title compound was prepared in accordance with Example 9 from 5-(4-tert-butylphenyl)-1-(4-cyclopentyloxyphenyl)indole-2-carbonyl chloride (see Example 1, step (d)) and 5-aminotetrazole.

200 MHz ¹H-NMR (DMSO-d₆, ppm) δ 12.6-12.5 (1H, br s) 8.02 (1H, s) 7.82 (1H, s) 7.67-7.53 (3H, m) 7.51-7.40 (2H, m) 7.35-7.24 (2H, m) 7.10 (1H, d, J=8.8 Hz) 7.07-6.95 (2H, m) 4.91-4.79 (1H, m) 2.05-1.49 (8H, m) 1.29 (9H, m).

### Example 11

### 5-(4-tert-Butylphenyl)-1-(4-cyclopentyloxyphenyl)indole-2-carboxylic acid(5-trifluoromethyl[1,3,4]thiadiazol-2-yl)amide

The title compound was prepared in accordance with Example 9 from 5-(4-tert-butylphenyl)-1-(4-cyclopentyloxyphenyl)indole-2-carbonyl chloride (see Example 1, step (d)) and 2-amino-5-trifluoromethyl[1,3,4]thiadiazole.

200 MHz ¹H-NMR (DMSO-d₆, ppm) δ 13.9-13.7 (1H, br s) 8.04 (1H, s) 7.97 (1H, s) 7.68-7.55 (3H, m) 7.51-7.41 (2H, m) 7.33-7.24 (2H, m) 7.10 (1H, d, J=8.8 Hz) 7.06-6.96 (2H, m) 4.94-4.81 (1H, m) 2.07-1.50 (8H, m) 1.30 (9H, m).

### Example 12

### 5-(4-tert-Butylphenyl)-1-(4-cyclopentyloxyphenyl)indole-2-carboxylic acid[1,2,4]triazol-4-ylamide

The title compound was prepared in accordance with Example 9 from 5-(4-tert-butylphenyl)-1-(4-cyclopentyloxyphenyl)indole-2-carbonyl chloride (see Example 1, step (d)) and 4-amino[1,2,4]triazole.

200 MHz ¹H-NMR (DMSO-d₆, ppm) δ 12.2-12.1 (1H, br s) 8.74 (2H, s) 8.05 (1H, s) 7.86-7.71 (6H, m) 7.32-7.21 (2H, m) 7.12 (1H, d, J=8.8 Hz) 7.05-6.95 (2H, m) 4.92-4.80 (1H, m) 2.06-1.48 (8H, m) 1.30 (9H, m).

### Example 13

### N-[3-Chloro-1-(4-isopropoxyphenyl)-5-(5-trifluoromethylpyridin-2-yl)indole-2-carbonyl]methanesulfonamide

### (a) 5-Bromo-3-chloroindole-2-carboxylic acid ethyl ester

A mixture of 5-bromoindole-2-carboxylic acid ethyl ester (4.00 g, 14.9 mmol), SO₂Cl₂ (1.8 mL, 22.4 mmol) and benzene (125 mL) was stirred at 90° C. for 2.5 h and cooled to rt. NaHCO₃ (aq, sat) was added and the mixture was extracted with EtOAc. The combined extracts were washed with water and brine, dried (Na₂SO₄), concentrated and crystallised from toluene to give the sub-title compound (3.87 g 85%).

### (b) 5-Bromo-3-chloro-1-(4-isopropoxyphenyl)indole-2-carboxylic acid ethyl ester

The sub-title compound was prepared in accordance with Example 1, step (b), Method B, using 5-bromo-3-chloroindole-2-carboxylic acid ethyl ester (see step (a) above) and 4-isopropoxyphenylboronic acid.

### (c) 3-Chloro-5-iodo-1-(4-isopropoxyphenyl)indole-2-carboxylic acid ethyl ester

A mixture of 5-bromo-3-chloro-1-(4-isopropoxyphenyl)indole-2-carboxylic acid ethyl ester (2.80 g, 6.44 mmol) (step (b) above), CuI (122 mg, 0.64 mmol), NaI (1.94 g, 12.9 mmol), N,N′-dimethyl-1,2-diaminoethane (142 μL, 1.28 mmol) and dioxane (10 mL) was stirred at 120° C. for 24 h. The mixture was cooled to rt, diluted with EtOAc (200 mL), washed with NH₄OH (aq), HCl (aq, 0.1 M) and brine, dried (Na₂SO₄) and concentrated to give the sub-title compound (3.02 g 97%).

### (d) 3-Chloro-5-(dihydroxyboryl)-1-(4-isopropoxyphenyl)indole-2-carboxylic acid ethyl ester

i-PrMgCl.LiCl (0.95 M in THF, 3.26 mL, 3.1 mmol) was added over 5 min to 3-chloro-5-iodo-1-(4-isopropoxyphenyl)indole-2-carboxylic acid ethyl ester (1.45 g, 3.0 mmol, see step (c) above) in THF (9 mL) at −40° C. After 15 min at −40° C., B(OEt)₃ (1.56 mL, 9.0 mmol) was added. The temperature was allowed to reach 0° C. over 2 h and HCl (aq, 2.5 M, 14.4 mL, 36 mmol) was added. After 1 h at 0° C., the mixture was diluted with brine (70 mL) and extracted with t-BuOMe (4×70 mL). The combined extracts were washed with brine (100 mL), dried (Na₂SO₄) and concentrated. The solid residue was treated several times with petroleum ether and filtered to give the sub-title compound (1.04 g, 86%)

### (e) 3-Chloro-1-(4-isopropoxyphenyl)-5-(5-trifluoromethylpyridin-2-yl)indole-2-carboxylic acid ethyl ester

A mixture of 3-chloro-5-(dihydroxyboryl)-1-(4-isopropoxyphenyl)indole-2-carboxylic acid ethyl ester (200 mg, 0.50 mmol; see step (d) above), 2-bromo-5-trifluoromethylpyridine (170 mg, 0.75 mmol), Na₂CO₃ (aq, 2 M, 0.75 mL, 1.5 mmol), Pd(PPh₃)₄ (29 mg, 0.025 mmol), EtOH (0.4 mL) and toluene (1.6 mL) was heated at 85° C. for 3 h. The mixture was diluted with EtOAc, washed with brine, dried (MgSO₄), concentrated and purified by chromatography to give the sub-title compound (239 mg, 95%).

### (e) 3-Chloro-1-(4-isopropoxyphenyl)-5-(5-trifluoromethylpyridin-2-yl)indole-2-carboxylic acid

The sub-title compound was prepared by hydrolysis of 3-chloro-1-(4-isopropoxyphenyl)-5-(5-trifluoromethylpyridin-2-yl)indole-2-carboxylic acid ethyl ester (see step (e) above) in accordance with the procedure in Example 1 (c).

### (g) 3-Chloro-1-(4-isopropoxyphenyl)-5-(5-trifluoromethylpyridin-2-yl)indole-2-carbonyl chloride

The sub-title compound was prepared in accordance with Example 1, step (d) from 3-chloro-1-(4-isopropoxyphenyl)-5-(5-trifluoromethylpyridin-2-yl)indole-2-carboxylic acid (see step (f) above).

### (h) N-[3-Chloro-1-(4-isopropoxyphenyl)-5-(5-trifluoromethylpyridin-2-yl)indole-2-carbonyl]methanesulfonamide

The title compound was prepared in accordance with Example 1, step (e) from 3-chloro-1-(4-isopropoxyphenyl)-5-(5-trifluoromethylpyridin-2-yl)indole-2-carbonyl chloride (see step (g) above) and methanesulfonamide.

200 MHz ¹H-NMR (DMSO-d₆, ppm) δ 12.5 (1H, br s) 9.05 (1H, s) 8.53 (1H, d, J=1.3 Hz) 8.38-8.18 (3H, m) 7.40 (1H, d, J=8.8 Hz) 7.39-7.29 (2H, m) 7.16-7.06 (2H, m) 4.69 (1H, septet, J=6.0 Hz) 3.21 (3H, s) 1.31 (6H, d, J=6.0 Hz).

### Example 14

### 3-Chloro-1-(4-isopropoxyphenyl)-5-(5-trifluoromethylpyridin-2-yl)indole-2-carboxylic acid (tetrazol-5-yl)amide

The title compound was prepared in accordance with Example 9 from 3-chloro-1-(4-isopropoxyphenyl)-5-(5-trifluoromethylpyridin-2-yl)indole-2-carbonyl chloride (see Example 13, step (g)) and 5-aminotetrazole.

200 MHz ¹H-NMR (DMSO-d₆, ppm) δ 16.4-16.0 (1H, br s) 13.0-12.7 (1H, br s) 9.07 (1H, s) 8.57 (1H, s) 8.42-8.21 (3H, m) 7.45-7.34 (3H, m) 7.14-7.04 (2H, m) 4.67 (1H, septet, J=6.0 Hz) 1.30 (6H, d, J=6.0 Hz).

### Example 15

### {[3-Chloro-1-(4-isopropoxyphenyl)-5-(5-trifluoromethylpyridin-2-yl)indole-2-carbonyl]amino}acetic acid

### (a) {[3-Chloro-1-(4-isopropoxyphenyl)-5-(5-trifluoromethylpyridin-2-yl)indole-2-carbonyl]amino}acetic acid methyl ester

The sub-title compound was prepared in accordance with Example 9 from 3-chloro-1-(4-isopropoxyphenyl)-5-(5-trifluoromethylpyridin-2-yl)indole-2-carbonyl chloride (see Example 13, step (g)) (see Example I, step (d)) and aminoacetic acid methyl ester hydrochloride.

### (b) {[3-Chloro-1-(4-isopropoxyphenyl)-5-(5-trifluoromethylpyridin-2-yl)indole-2-carbonyl]amino}acetic acid

The title compound was prepared by hydrolysis in accordance with Example 1, step (c) and purification by chromatography from {[3-chloro-1-(4-isopropoxyphenyl)-5-(5-trifluoromethylpyridin-2-yl)indole-2-carbonyl]-amino}acetic acid methyl ester (see step (a) above).

200 MHz ¹H-NMR (DMSO-d₆, ppm) δ 13.0-12.5 (1H, br s) 9.02 (1H, s) 8.83 (1H, t, J=5.7 Hz) 8.47 (1H, s) 8.34-8.19 (2H, m) 8.15 (1H, dd, J=9.0, 1.4 Hz) 7.40-7.28 (3H, m) 7.07-6.96 (2H, m) 4.65 (1H, septet, J=6.0 Hz) 3.81 (2H, d, J=5.7 Hz) 1.30 (6H, d, J=6.0 Hz).

### Example 16

### 1-(4-Isopropoxyphenyl)-5-[(5-trifluoromethylpyrid-2-yl)oxy]indole-2-carboxylic acid 2-aminoethylamide dihydrochloride

### (a) 5-Benzyloxy-1-(4-isopropoxyphenyl)indole-2-carboxylic acid ethyl ester

A stock solution was prepared from CuI (76.2 mg, 0.4 mmol), N,N′-dimethylethylene diamine (85 μL, 0.8 mmol) and anhydrous toluene (4 mL). 4-Isopropoxyphenylbromide (0.15 g, 0.7 mmol) in toluene (1 mL) followed by 1.2 mL of the stock solution was added to K₃PO₄ (0.22 g, 1.05 mmol) and 5-hydroxyindole-2-carboxylic acid ethyl ester (0.15 g, 0.5 mmol) under argon. The mixture was stirred at 110-120° C. for 20, allowed to cool and filtered. The precipitate was washed with acetone and the filtrates were concentrated and purified by chromatography to give the sub-title compound (163 mg, 75%).

### (b) 5-Hydroxy-4-(1-isopropoxyphenyl)indole-2-carboxylic acid ethyl ester

Hydrogen was bubbled through a mixture of 5-benzyloxy-1-(4-isopropoxyphenyl)indole-2-carboxylic acid ethyl ester (1.00 g, 2.3 mL, see step (a) above), Pd—C (10%, 0.45 g), HCl (aq, conc, 0.21 mL), H₂O (0.21 mL) and EtOAc (15 mL). After completion of the reaction, as judged by TLC, the mixture was filtered, concentrated and purified by chromatography. The material was treated with petroleum ether to give a solid which was collected to give the sub-title compound (0.70 g, 88%), m.p. 140-141° C.

### (c) 1-(4-Isopropoxyphenyl)-5-[(5-trifluoromethylpyrid-2-yl)oxy]indole-2-carboxylic acid ethyl ester

A mixture of 5-hydroxy-1-(4-isopropoxyphenyl)indole-2-carboxylic acid ethyl ester (0.40 g, 1.18 mmol, see step (b) above), K₂CO₃ (1.30 g, 9.44 mmol), 2-chloro-5-trifluoromethylpyridine (0.43 g, 2.36 mmol), 18-crown-6 (27 mg) and DMF (3 mL) was heated at 55° C. for 15 h. CH₂Cl₂ and H₂O were added and the organic layer was collected, concentrated and purified by chromatography to give the sub-title compound (0.50 g, 87%).

### (d) 1-(4-Isopropoxyphenyl)-5-[(5-trifluoromethylpyrid-2-yl)oxy]indole-2-carboxylic acid

NaOH (1 M in MeOH, 4 mL) was added to 1-(4-isopropoxyphenyl)-5-[(5-tri-fluoromethylpyrid-2-yl)oxy]-indole-2-carboxylic acid ethyl ester (0.35 g, 0.72 mmol, see step (c) above) in CH₂Cl₂ (3 mL) at rt. The mixture was heated at 50° C. for 6 h, stirred overnight at rt, heated at reflux for 3 h, cooled and acidified to pH 2-3 with HCl (aq, conc). CH₂Cl₂ and H₂O were added and the organic layer was washed twice with H₂O, dried (Na₂SO₄), concentrated and crystallised from acetone/H₂O to give the sub-title compound (0.28 g, 85%) as a slightly yellowish solid.

### (e) 1-(4-Isopropoxyphenyl)-5-[(5-trifluoromethylpyrid-2-yl)oxy]-indole-2-carboxylic acid 2-tert-butyloxycarbonylaminoethylamide

A mixture of 1-(4-isopropoxyphenyl)-5-[(5-trifluoromethylpyrid-2-yl)oxy]indole-2-carboxylic acid (0.27 g, 0.6 mmol, see step (d) above), N-tert-butyloxycarbonyl-ethylendiamine (0.12 g, 0.6 mmol), 1-(3-dimethylaminopropyl)-3-ethylcarbodiimide hydrochloride (0.12 g, 0.6 mmol), 1-hydroxybenzotriazol (0.08 g, 0.6 mmol), Et₃N (0.17 mL, 1.2 mmol) and MeCN (6 mL) was stirred at 30-35° C. for 24 h. CH₂Cl₂ and H₂O were added and the organic layer was washed with H₂O, dried (Na₂SO₄), concentrated and crystallised from acetone/water. The material was purified by chromatography and crystallised from CH₂Cl₂/hexane to give the sub-title compound (0.18 g, 51%) as a white solid. Mp 188-190° C.

### (f) 1-(4-Isopropoxyphenyl)-5-[(5-trifluoromethylpyrid-2-yl)oxy]indole-2-carboxylic acid 2-aminoethylamide dihydrochloride

HCl (1 M in MeOH, 3 mL) was added to 1-(4-isopropoxyphenyl)-5-[(5-trifluoromethylpyrid-2-yl)oxy]-indole-2-carboxylic acid 2-tert-butyloxycarbonylaminoethylamide (68 mg, 0.11 mmol) in CH₂Cl₂. The mixture was stirred at 35-40° C. for 6 h, at rt for 18 h and again at 35-40° C. for 6 h. The mixture was concentrated and purified by chromatography to give a material which was crystallised from EtOAc/hexane to give the title compound (30 mg, 49%) as a yellowish powder. Mp 114-116° C.

200 MHz ¹H-NMR (CDCl₃, ppm) δ 8.75 (1H, d) 8.54 (1H, d) 8.22 (1H, dd, J 8.8, 2.2 Hz) 8.05-7.85 (3H, br s) 7.57 (1H, s) 7.34-7.17 (4H, m) 7.11-6.97 (4H, m) 4.68 (1H, septet, J=6.0 Hz) 3.50-3.30 (2H, m) 2.93 (2H, t) 1.33 (6H, d, J=6.0 Hz).

### Example 17

### 3-Chloro-1-(4-isopropoxyphenyl)-5-[(5-trifluoromethylpyrid-2-yl)oxy]indole-2-carboxylic acid 2-aminoethylamide dihydrochloride

### (a) 3-Chloro-1-(4-isopropoxyphenyl)-5-[(5-trifluoromethylpyrid-2-yl)oxy]indole-2-carboxylic acid 2-tert-butyloxycarbonylaminoethylamide

SO₂Cl₂ (27 mg, 0.20 mmol) in CH₂Cl₂ (1 mL) was added to 1-(4-isopropoxyphenyl)-5-[(5-trifluoromethylpyrid-2-yl)oxy]indole-2-carboxylic acid 2-butyloxycarbonylaminoethylamide (0.11 g, 0.18 mmol, see step (e) Example 16) in anhydrous CH₂Cl₂ (5 mL). After 3.5 h at rt additional SO₂Cl₂ (5 mg, 0.04 mmol) was added and the mixture was stirred for 30 min at rt. The mixture was poured into NaHCO₃ (aq, sat). CH₂Cl₂ was added and the organic layer was washed with H₂O, dried (Na₂SO₄) and concentrated. The residue was treated with petroleum ether to give the sub-title compound as a white solid (0.10 g, 83%). Mp 184-185° C.

### (b) 3-Chloro-1-(4-isopropoxyphenyl)-5-[(5-trifluoromethylpyrid-2-yl)oxy]indole-2-carboxylic acid 2-aminoethylamide dihydrochloride

HCl (1 M in MeOH, 3 mL) was added to 3-chloro-1-(4-isopropoxyphenyl)-5-[(5-trifluoromethylpyrid-2-yl)oxy]indole-2-carboxylic acid 2-tert-butyloxycarbonylaminoethylamide (0.10 g, 0.16 mmol) in CH₂Cl₂ (2 mL). The mixture was stirred at rt for 45 h, concentrated and purified by chromatography. Crystallisation from acetone/hexane gave the title compound (58 mg, 64%) as a yellowish powder. Mp 131-134° C.

200 MHz ¹H-NMR (DMSO-d₆, ppm) δ 8.92 (1H, t) 8.55 (1H, s) 1.32 (6H, d, J=5.8 Hz); 8.23 (1H, dd, J=8.5 Hz) 8.2-8.0 (3H, br s) 7.46 (1H, d) 7.36 (2H, d, J=8.3 Hz) 7.31-7.21 (2H, m) 7.07 (2H, d, J=8.3 Hz) 4.69 (1H, septet, J=5.8 Hz) 3.53-3.35 (2H, m, overlapped with DMSO and water) 2.88-2.72 (2H, m).

### Example 18

### 2-[(1-Carboxycyclopropylcarbamoyl)methyl]-1-(4-isopropoxyphenyl)-5-(4-trifluoromethylphenoxy)indole-3-carboxylic acid

### (a) 3-(4-Isopropoxyphenylamino)pent-2-enedioic acid diethyl ester

A mixture of 4-isopropoxyaniline (8.5 g, 56.2 mmol), diethyl-1,3-acetonedicarboxylate (10.2 mL, 56.2 mmol), a catalytic amount of p-toluenesulfonic acid and CHCl₃ (50 mL) was heated at refluxed for 6 h while the water that formed was removed with a Dean-Stark trap. The mixture was concentrated and the sub-title compound was used in the subsequent step without further purification.

### (b) 2-Ethoxycarbonylmethyl-5-hydroxy-1-(4-isopropoxyphenyl)indole-3-carboxylic acid ethyl ester

A mixture of 3-(4-isopropoxyphenylamino)pent-2-enedioic acid diethyl ester (9.0 g, 26.8 mmol, see step (a) above), 1,4-benzoquinone (3.6 g, 33.5 mmol) and anhydrous MeCN (50 mL) was stirred at 70° C. for 2 d and left at 4° C. for 1 d. The precipitate was filtered off and recrystallised from MeCN to give the sub-title compound (3.8 g, 33%).

### (c) 2-Ethoxycarbonylmethyl-1-(4-isopropoxyphenyl)-5-(4-trifluoromethylphenoxy)indole-3-carboxylic acid ethyl ester

A mixture of 2-ethoxycarbonylmethyl-5-hydroxy-1-(4-isopropoxyphenyl)indole-3-carboxylic acid ethyl ester (250 mg, 0.59 mmol, see step (b) above), Et₃N (106 mg, 1.05 mmol), pyridine (84 mg, 1.05 mmol), Cu(OAc)₂ (107 mg, 0.59 mmol), 4-trifluoromethylphenylboronic acid (167 mg, 0.88 mmol) and CH₂Cl₂ (10 mL) was stirred at rt for 2 d, filtered through Celite®, concentrated and purified by chromatography to give the sub-title compound, which was used in the subsequent step without further purification.

### (d) 2-Carboxymethyl-1-(4-isopropoxyphenyl)-5-(4-trifluoromethylphenoxy)indole-3-carboxylic acid ethyl ester

NaOH (aq, 2 M, 2.5 mL) was added to 2-ethoxycarbonylmethyl-1-(4-isopropoxyphenyl)-5-(4-trifluoromethylphenoxy)indole-3-carboxylic acid ethyl ester (265 mg, 0.465 mmol, see step (c) above) in EtOH (15 mL). The mixture was stirred at rt for 16 h, acidified to pH 2-3 with HCl (aq, conc), concentrated ands purified by chromatography to give the sub-title compound (176 mg, 70%).

### (e) 2-[(1-Ethoxycarbonylcyclopropylcarbamoyl)methyl]-1-(4-isopropoxyphenyl)-5-(4-trifluoromethylphenoxy)indole-3-carboxylic acid ethyl ester

Et₃N (54 mg, 0.532 mmol) was added to a mixture of 2-carboxymethyl-1-(4-iso*propoxyphenyl)-5-(4-trifluoromethylphenoxy)indole-3-carboxylic acid ethyl ester (144 mg, 0.266 mmol, see step (d) above), 1-amino-1-cyclopropanecarboxylic acid ethyl ester hydrochloride (44 mg, 0.266 mmol), HBTU (101 mg, 0.266 mmol) and anhydrous MeCN (10 mL). The mixture was stirred at rt for 24 h. NaHCO₃ (aq, sat, 10 mL) was added and the mixture was extracted with EtOAc. The organic layer was washed with saturated NaHCO₃ (aq, sat, 10 mL) and brine (15 mL), dried (Na₂SO₄) and concentrated to give the sub-title compound which was used in the next step without further purification.

### (f) 2-[(1-Carboxycyclopropylcarbamoyl)methyl]-1-(4-isopropoxyphenyl)-5-(4-trifluoromethylphenoxy)indole-3-carboxylic acid dihydrate

Dioxane (4 mL) was added to a suspension of 2-[(1-ethoxycarbonylcyclopropylcarbamoyl)methyl]-1-(4-isopropoxyphenyl)-5-(4-trifluoromethylphenoxy)indole-3-carboxylic acid ethyl ester (170 mg, 0.266 mmol, see step (e) above) in NaOH (aq, 2 M, 4 mL). The mixture was heated at reflux for 3 h, cooled and acidified to pH 2-3 with HCl (aq, sat). H₂O was added and the precipitate was collected, washed with H₂O and recrystallised from EtOH to give the title compound (110 mg, 70%) as a white powder. Mp 220° C.

200 MHz ¹H-NMR (DMSO-d₆, ppm) δ 12.5-12.1 (2H, br s) 8.41 (1H, s) 7.76 (1H, dd, J=2.1 Hz) 7.74-7.65 (2H, m) 7.39-7.29 (2H, m) 7.18-7.02 (5H, m) 6.98 (1H, dd, J=8.9 Hz and 2.1 Hz) 4.70 (1H, septet, J=6.0 Hz) 3.93 (2H, s) 1.33 (6H, d, J=6.0 Hz) 1.25 (2H, dd, J=7.3 and 4.0 Hz) 0.79 (2H, dd, J=7.3 and 4.0 Hz).

### Example 19

### 5-(4-tert-Butylphenoxy)-2-[(1-carboxycyclopropylcarbamoyl)methyl]-1-(4-isopropoxyphenyl)-indole-3-carboxylic acid

### (a) 5-(4-tert-Butylphenoxy)-2-ethoxycarbonylmethyl-1-(4-isopropoxyphenyl)indole-3-carboxylic acid ethyl ester

The sub-title compound was prepared in accordance with Example 18, step (c) from 2-ethoxycarbonylmethyl-5-hydroxy-1-(4-isopropoxyphenyl)indole-3-carboxylic acid ethyl ester (250 mg, 0.59 mmol, see step (b) Example 18), and 4-tert-butylphenylboronic acid (157 mg, 0.88 mmol).

### (b) 5-(4-tert-Butylphenoxy)-2-carboxymethyl-1-(4-isopropoxyphenyl)indole-3-carboxylic acid ethyl ester

The sub-title compound was prepared in accordance with Example 18, step (d) from 5-(4-tert-butylphenoxy)-2-ethoxycarbonylmethyl-1-(4-isopropoxyphenyl)indole-3-carboxylic acid ethyl ester (290 mg, 0.524 mmol, see step (a) above).

### (c) 5-(4-tert-Butylphenoxy)-2-[(1-ethoxycarbonylcyclopropylaminocarbonyl)methyl]-1-(4-isopropoxyphenyl)-indole-3-carboxylic acid ethyl ester

The sub-title compound was prepared in accordance with Example 18, step (e) from 5-(4-tert-butylphenoxy)-2-carboxymethyl-1-(4-isopropoxyphenyl)indole-3-carboxylic acid ethyl ester (100 mg, 0.189 mmol, see step (b) above).

### (d) 5-(4-tert-Butylphenoxy)-2-[(1-carboxycyclopropylcarbamoyl)methyl]-1-(4-isopropoxyphenyl)-indole-3-carboxylic acid

The sub-title compound was prepared in accordance with Example 18, step (f) from 5-(4-tert-butylphenoxy)-2-[(1-ethoxycarbonylcyclopropylaminocarbonyl)methyl]-1-(4-isopropoxyphenyl)indole-3-carboxylic acid ethyl ester (130 mg, 0.203 mmol, see step (c) above) Yield 55 mg (46%) as a grey foam.

200 MHz ¹H-NMR (DMSO-d₆, ppm) δ 12.5-12.1 (2H, br s) 8.38 (1H, s) 7.68 (1H, d, J=2.1 Hz) 7.40-7.27 (4H, m) 7.14-7.03 (2H, m) 6.98 (1H, d, J=9.0 Hz) 6.93-6.83 (3H, m) 4.70 (1H, septet, J=6.0 Hz) 3.91 (2H, s) 1.33 (6H, d, J=6.0 Hz) 1.26 (9H, s) 1.27-1.19 (2H, m) 0.84-0.72 (2H, m).

### Example 20

### {[3-Chloro-1-(4-isopropoxyphenyl)-5-(4-trifluoromethylphenoxy)indole-2-carbonyl]amino}acetic acid

### (a) 1-(4-Isopropoxyphenyl)-5-(4-trifluoromethylphenoxy)indole-2-carboxylic acid ethyl ester

The sub-title compound was prepared in accordance with Example 18, step (c) from 2-ethoxycarbonylmethyl-5-hydroxy-1-(4-isopropoxyphenyl)indole-2-carboxylic acid ethyl ester (98 mg, 0.29 mmol, see step (b) Example 16) and 4-trifluoromethylphenylboronic acid (110 mg, 0.58 mmol). Yield 51 mg (51%).

### (b) 3-Chloro-1-(4-isopropoxyphenyl)-5-(4-trifluoromethylphenoxy]-indole-2-carboxylic acid ethyl ester

SO₂Cl₂ (0.23 g, 1.74 mmol) in anhydrous CH₂Cl₂ (6 mL) was added to 1-(4-isopropoxyphenyl)-5-(4-trifluoromethylphenoxy)-indole-2-carboxylic acid ethyl ester (0.65 g, 1.34 mmol, see step (a) above) in anhydrous CH₂Cl₂ (20 mL). The mixture was stirred at rt for 1.5 h and poured into NaHCO₃ (aq, sat). CH₂Cl₂ was added and the organic layer was washed twice with H₂O, dried (Na₂SO₄), concentrated and purified by crystallisation from CH₂Cl₂/hexane to give the sub-title compound (0.58 g, 83%) as a white solid. Mp 136-138° C.

### (c) 3-chloro-1-(4-isopropoxyphenyl)-5-(4-trifluoromethylphenoxy]indole-2-carboxylic acid

NaOH (1 M in MeOH, 4 mL, 4 mmol) and H₂O (0.6 mL) were added to 3-chloro-1-(4-isopropoxyphenyl)-5-(4-trifluoromethylphenoxy]-indole-2-carboxylic acid ethyl ester (0.56 g, 1.08 mmol, see step (b) above) in CH₂Cl₂ (11 mL)d at rt. The mixture was stirred at rt for 24 h and acidified to pH 2-3 with conc. HCl (aq, conc).CH₂Cl₂ and water were added and the organic layer was washed twice with H₂O, dried (Na₂SO₄), filtered, concentrated and treated with hexane. The precipitate was collected to give the sub-title compound (0.46 g, 87%) as a white solid with. Mp 148-150° C.

### (d) {[3-Chloro-1-(4-isopropoxyphenyl)-5-(4-trifluoromethylphenoxy)indole-2-carbonyl]amino}acetic acid ethyl ester

Glycine ethyl ester hydrochloride (28 mg, 0.20 mmol), Et₃N (61 μL, 0.60 mmol), and HBTU (72 mg, 0.20 mmol) were added to a mixture of 3-chloro-1-(4-isopropoxyphenyl)-5-(4-trifluoromethyl-phenoxy]indole-2-carboxylic acid (100 mg, 0.20 mmol, see step (c) above) in anhydrous MeCN (4 mL). The mixture was stirred at 40° C. for 24 h and concentrated. CH₂Cl₂ and H₂O were added and the organic layer was washed with H₂O, dried (Na₂SO₄), concentrated and purified by chromatography. Crystallisation from acetone/hexane gave the sub-title compound (71 mg, 61%) as a white solid with. Mp 80-81° C.

### (e) {[3-Chloro-1-(4-isopropoxyphenyl)-5-(4-trifluoromethylphenoxy)indole-2-carbonyl]amino}acetic acid

HCl (1 M in MeOH, 1 mL, 1 mmol) and H₂O n(3 drops) were added to {[3-chloro-1-(4-isopropoxyphenyl)-5-(4-trifluoromethylphenoxy)indole-2-carbonyl]amino}acetic acid ethyl ester (70 mg, 0.12 mmol, see step (d) above) in CH₂Cl₂ (1 mL). The mixture was stirred at rt for 2 h and acidified with HCl (aq, conc). CH₂Cl₂ and H₂O were added and the organic layer was washed twice with H₂O, dried (Na₂SO₄), and concentrated. The residue was treated with hexane which gave the title compound (59 mg, 88%) as a white solid with. Mp 94-96° C.

200 MHz ¹H-NMR (CDCl₃, ppm) δ 14.0-11.6 (1H, br s) 8.93 (1H, t, J=5.6 Hz) 7.72 (2H, d, J=8.2 Hz) 7.44-7.24 (4H, m) 7.20-6.95 (5H, m) 4.67 (1H, septet, J=6.0 Hz) 3.87 (2H, d, J=5.6 Hz) 1.31 (6H, d, J=6.0 Hz).

### Example 21

### {[3-Chloro-1-(4-isopropoxyphenyl)-5-(4-trifluoromethylphenoxy)indole-2-carbonyl]amino}-2-methylpropionic acid

### (a) {[3-Chloro-1-(4-isopropoxyphenyl)-5-(4-trifluoromethylphenoxy)indole-2-carbonyl]amino}-2-methylpropionic acid ethyl ester

2,2-Dimethylglycine ethyl ester hydrochloride (34 mg, 20 mmol), Et₃N (61 μL, 0.60 mmol) and HBTU (72 mg, 0.20 mmol) were added to a mixture of 3-chloro-1-(4-isopropoxyphenyl)-5-(4-trifluoromethylphenoxy]-indole-2-carboxylic acid (100 mg, 0.20 mmol, see step (c) Example 20) in anhydrous MeCN (4 mL). The mixture was stirred at rt for 63 h. CH₂Cl₂ and H₂O were added and the organic layer was washed with H₂O, dried (Na₂SO₄), concentrated and purified by chromatography. Crystallisation from CH₂Cl₂/hexane gave the sub-title compound (80 mg, 65%) as a slightly yellowish solid with. Mp 135-137° C.

### (b) {[3-Chloro-1-(4-isopropoxyphenyl)-5-(4-trifluoromethylphenoxy)indole-2-carbonyl]amino}-2-methylpropionic acid

The title compound was prepared in accordance with Example 20, step (e) by treating {[3-chloro-1-(4-isopropoxyphenyl)-5-(4-trifluoromethylphenoxy)indole-2-carbonyl]amino}-2-methylpropionic ethyl ester (80 mg, 0.13 mmol, see step (a) above) with HCl (1 M in MeOH, 1 mL, 1 mmol) for 4 h. Yield 44 mg (58%) from acetone/hexane as a yellowish powder. Mp 187-188° C.

200 MHz ¹H-NMR (CDCl₃, ppm) δ 12.5-12.1 (1H, br s) 8.85 (1H, s) 7.72 (2H, d, J=8.2 Hz) 7.40-7.29 (4H, m) 7.18-6.97 (5H, m) 4.68 (1H, heptet, J=6.0 Hz) 1.38-1.25 (12H, m).

### Example 22

Title compounds of the examples were tested in the biological test described above and were found to exhibit 50% inhibition of mPGES-1 at a concentration of 10 μmol or below. For example, the following representative compounds of the examples exhibited the following IC₅₀ values:

| | | |
|---|---|---|
| Example 2: | 660 | nM |
| Example 4: | 1900 | nM |
| Example 5: | 740 | nM |
| Example 12: | 550 | nM |
| Example 13: | 2700 | nM |

1. A compound of formula I, 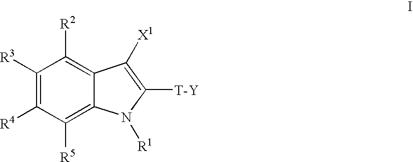
wherein
one of the groups R², R³, R⁴ and R⁵ represents -D-E and:
a) the other groups are independently selected from hydrogen, G¹, an aryl group, a heteroaryl group (which latter two groups are optionally substituted by one or more substituents selected from A), C₁₋₈ alkyl and a heterocycloalkyl group (which latter two groups are optionally substituted by one or more substituents selected from G¹ and/or Z¹); and/or
b) any two other groups which are adjacent to each other are optionally linked to form, along with two atoms of the essential benzene ring in the compound of formula I, a 3- to 8-membered ring, optionally containing 1 to 3 heteroatoms, which ring is itself optionally substituted by one or more substituents selected from halo, —R⁶, —OR⁶ and ═O;
D represents a single bond, —O—, —C(R⁷)(R⁸)—, C₂₋₄ alkylene, —C(O)— or —S(O)ₘ—;
R¹ and E independently represent an aryl group or a heteroaryl group, both of which groups are optionally substituted by one or more substituents selected from A;
R⁷ and R⁸ independently represent H, halo or C₁₋₆ alkyl, which latter group is optionally substituted by halo, or R⁷ and R⁸ may together form, along with the carbon atom to which they are attached, a 3- to 6-membered ring, which ring optionally contains a heteroatom and is optionally substituted by one or more substituents selected from halo and C₁₋₃ alkyl, which latter group is optionally substituted by one or more halo substituents;
X¹ represents H, halo, —N(R^{9k})-J-R^{10k}, —C(O)OR^{9a}, —C(O)N(R^{10b})R^{9b}, —S(O)₂N(R^{10b})R^{9b}, —C(O)N(H)C(═NR^{9c})N(R^{10d})R^{9d}—C(O)N(H)CN S(O)₃R^{9e}, —P(O)(OR^{9f})₂, —P(O)(OR^{9g})N(R^{10h})R^{9h}—P(O)(N(R¹⁰ⁱ)R⁹ⁱ)₂, —B(OR^{9j})₂, —C(O)N(H)S(O)₂R¹¹ or -Q-X²;
J represents a single bond, —C(O)— or —S(O)ₘ—;
Q represents a single bond, —O—, —C(O)—, —S(O)ₘ— or a C₁₋₈ alkylene or C₂₋₈ heteroalkylene chain, both of which latter two groups optionally contain one or more unsaturations and are optionally substituted by one or more substituents selected from G¹, Z¹ and/or X³;
X² represents:
(a) an aryl group or a heteroaryl group, both of which are optionally substituted by one or more substituents selected from A and/or X³; or
(b) C₁₋₈ alkyl, C₂₋₈ heteroalkyl or a heterocycloalkyl group, all of which are optionally substituted by one or more substituents selected from G¹, Z¹ and/or X³;
X³ represents —C(O)OR^{9a}, —C(O)N(R^{10b})R^{9b}, —C(O)N(H)C(═NR^{9c})N(R^{10d})R^{9d}, —C(O)N(H)CN, —S(O)₃R^{9e}, —P(O)(OR^{9f})₂, —P(O)(OR^{9g})N(R^{10h})R^{9h}, —P(O)(N(R¹⁰ⁱ)R⁹ⁱ)₂, —B(OR^{9j})₂ or —C(O)N(H)S(O)₂R¹¹;
T represents:
(a) a single bond;
(b) a C₁₋₈ alkylene or a C₂₋₈ heteroalkylene chain, both of which latter two groups:
(i) optionally contain one or more unsaturations;
(ii) are optionally substituted by one or more substituents selected from G¹ and/or Z¹; and/or
(iii) may comprise an additional 3- to 8-membered ring formed between any one or more members of the C₁₋₈ alkylene or C₂₋₈ heteroalkylene chain, which ring optionally contains 1 to 3 heteroatoms and/or 1 to 3 unsaturations and which ring is itself optionally substituted by one or more substituents selected from G¹ and/or Z¹;
(c) an arylene group or a heteroarylene group, both of which groups are optionally substituted by one or more substituents selected from A; or
(d) -T¹-W¹-T²-;
one of T¹ and T² represents a C₁₋₈ alkylene or a C₂₋₈ heteroalkylene chain, both of which latter two groups:
(i) optionally contain one or more unsaturations;
(ii) are optionally substituted by one or more substituents selected from G¹ and/or Z¹; and/or
(iii) may comprise an additional 3- to 8-membered ring formed between any one or more members of the C₁₋₈ alkylene or C₂₋₈ heteroalkylene chain, which ring optionally contains 1 to 3 heteroatoms and/or 1 to 3 unsaturations and which ring is itself optionally substituted by one or more substituents selected from G¹ and/or Z;
and the other represents an arylene group or a heteroarylene group, both of which groups are optionally substituted by one or more substituents selected from A;
W¹ represents —O— or —S(O)ₘ—;
m represents 0, 1 or 2;
Y represents —C(O)N(R^{10b})R^{9b}, —C(O)N(H)C(═NR^{9c})N(R^{10d})R^{9d}—C(O)N(H)CN or —C(O)N(H)S(O)₂R¹¹;
R⁶, R^{9a} to R^{9k}, R^{10b}, R^{10d}, R^{10h}, R¹⁰ⁱ and R^{10k} independently represent:
I) hydrogen;
II) an aryl group or a heteroaryl group, both of which are optionally substituted by one or more substituents selected from B; or
III) C₁₋₈ alkyl or a heterocycloalkyl group, both of which are optionally substituted by one or more substituents selected from G¹ and/or Z¹; or
any pair of R^{9a} to R^{9k} and R^{10b}, R^{10d}, R^{10h}, R¹⁰ⁱ or R^{10k}, may be linked together to form, along with the atom(s) and/or group(s) to which they are attached, a 3- to 8-membered ring, optionally containing 1 to 3 heteroatoms and/or 1 to 3 double bonds, which ring is optionally substituted by one or more substituents selected from G¹ and/or Z¹;
R¹¹ represents:
I) an aryl group or a heteroaryl group, both of which are optionally substituted by one or more substituents selected from B; or
II) C₁₋₈ alkyl or a heterocycloalkyl group, both of which are optionally substituted by one or more substituents selected from G¹ and/or Z¹;
A represents:
I) an aryl group or a heteroaryl group, both of which are optionally substituted by one or more substituents selected from B;
II) C₁₋₈ alkyl or a heterocycloalkyl group, both of which are optionally substituted by one or more substituents selected from G¹ and/or Z¹; or
III) a G¹ group;
G¹ represents halo, cyano, —N₃, —NO₂, —ONO₂ or -A¹-R^{12a}.
wherein A¹ represents a single bond or a spacer group selected from —C(O)A²-, —S(O)₂A³-, —N(R^{13a})A⁴ or -OA⁵-, in which:
A represents a single bond, —O—, —N(R^{3b})— or —C(O)—;
A³ represents a single bond, —O— or —N(R^{13c})—;
A⁴ and A⁵ independently represent a single bond, —C(O)—, —C(O)N(R^{13d})—, —C(O)O—, —S(O)₂— or —S(O)₂N(R^{13d})—;
Z¹ represents ═O, ═S, ═NOR^{12b}, ═NS(O)₂N(R^{13f})R^{12c}, ═NCN or ═C(H)NO₂;
B represents:
I) an aryl group or a heteroaryl group, both of which are optionally substituted by one or more substituents selected from G²;
II) C₁₋₈ alkyl or a heterocycloalkyl group, both of which are optionally substituted by one or more substituents selected from G² and/or Z²; or
III) a G² group;
G² represents halo, cyano, —N₃, —NO₂, —ONO₂ or -A⁶-R^{14a};
wherein A⁶ represents a single bond or a spacer group selected from —C(O)A⁷-, —S(O)₂A⁸-, —N(R^{15a})A⁹- or —OA¹⁰-, in which:
A⁷ represents a single bond, —O—, —N(R^{15b})— or —C(O)—;
A⁸ represents a single bond, —O— or —N(R^{15c})—;
A⁹ and A¹⁰ independently represent a single bond, —C(O)—, —C(O)N(R^{15d})—, —C(O)O—, —S(O)₂— or —S(O)₂N(R^{15e})—;
Z² represents ═O, ═S, ═NOR^{14b}, ═NS(O)₂N(R^{15f})R^{14c}, ═NCN or ═C(H)NO₂;
R^{12a}, R^{12b}, R^{12c}, R^{13a}, R^{13b}, R^{13c}, R^{13d}, R^{13e}, R^{13f}, R^{14a}, R^{14b}, R^{14c}, R^{15a}, R^{15b}, R^{15c}, R^{15d}, R^{15e} and R^{15f} are independently selected from:
i) hydrogen;
ii) an aryl group or a heteroaryl group, both of which are optionally substituted by one or more substituents selected from G³;
iii) C₁₋₈ alkyl or a heterocycloalkyl group, both of which are optionally substituted by G³ and/or Z³; or
any pair of R^{12a} to R^{12c} and R^{3a} to R^{13f} and/or R^{14a} to R^{14c} and R^{15a} to R^{15f}, may be linked together to form with those, or other relevant, atoms a further 3- to 8-membered ring, optionally containing 1 to 3 heteroatoms and/or 1 to 3 double bonds, which ring is optionally substituted by one or more substituents selected from G³ and/or Z³;
G³ represents halo, cyano, —N₃, —NO₂, —ONO₂ or -A¹¹-R^{16a};
wherein A¹¹ represents a single bond or a spacer group selected from —C(O)A¹²-, —S(O)₂A¹³-, —N(R^{17a})A¹⁴ or —OA¹⁵-, in which:
A¹² represents a single bond, —O—, —N(R^{17b}) or —C(O)—;
A¹³ represents a single bond, —O— or —N(R^{7c})—;
A 14 and A¹⁵ independently represent a single bond, —C(O)—, —C(O)N(R^{7d})—, —C(O)O—, —S(O)₂— or —S(O)₂N(R^{17e})—;
Z³ represents ═O, ═S, ═NOR^{16b}, ═NS(O)₂N(R^{17f})R^{16c}, ═NCN or ═C(H)NO₂;
R^{16a}, R^{16b}, R^{16c}, R^{17a}, R^{17b}, R^{17c}, R^{17d}, R^{17e}, R^{17f} are independently selected from:
i) hydrogen;
ii) C₁₋₆ alkyl or a heterocycloalkyl group, both of which groups are optionally substituted by one or more substituents selected from halo, C₁₋₄ alkyl, —N(R^{18a})R^{19a}, —OR^{18b} and ═O; and
iii) an aryl or heteroaryl group, both of which are optionally substituted by one or more substituents selected from halo, C₁₋₄ alkyl, —N(R^{8c})R^{19b} and —OR^{18d}; or
any pair of R¹⁶ to R^{16c} and R^{17a} to R^{17f} may be linked together to form with those, or other relevant, atoms a further 3- to 8-membered ring, optionally containing 1 to 3 heteroatoms and/or 1 to 3 double bonds, which ring is optionally substituted by one or more substituents selected from halo, C₁₋₄ alkyl, —N(R^{18e})R^{19c}, —OR^{18f} and ═O;
R^{18a}, R^{18b}, R^{18c}, R^{18d}, R^{18e}, R^{18f}, R^{19a}, R^{19b} and R^{19c} are independently selected from hydrogen and C₁₋₄ alkyl, which latter group is optionally substituted by one or more halo groups;
or a pharmaceutically-acceptable salt thereof,
provided that, when R¹ represents 3,4-dimethoxyphenyl, T both represent single bonds, X¹, R², R⁴ and R⁵ all represent H, R³ represents -D-E, in which D represents a single bond and E represents phenyl, or D represents —O— and E represents 4-chlorophenyl, and Y represents —C(O)N(R^{10b})R^{9b}, then R^{9b} and R^{10b} are not linked together to form, along with the N atom to which they are attached, a 4-morpholin-1-yl ring.

2. A compound as claimed in claim 1, wherein X¹ represents H, halo, —N(R^{9k})-J-R^{10k}, —C(O)OR^{9a}, —C(O)N(R^{10b})R^{9b}, —C(O)N(H)C(═NR^{9c})N(R^{10d})R^{9d}, —C(O)N(H)CN, —S(O)₃R^{9e}, —P(O)(OR^{9f})₂, —P(O)(OR^{9g})N(R^{10h})R^{9h}, —P(O)(N(R¹⁰ⁱ)R⁹ⁱ)₂, —B(OR^{9j})₂, —C(O)N(H)S(O)₂R¹¹ or -QX²

3. A compound as claimed in claim 1 or claim 2, wherein A represents G¹ or C₁₋₆ alkyl optionally substituted by one or more G¹ groups.

4. A compound as claimed in claim 1, wherein G¹ represents fluoro, chloro or -A¹-R^{12a}.

5. A compound as claimed in claim 1, wherein A¹ represents —C(O)A²-, —S(O)₂A³-, —N(R^{13a})A⁴- or —OA⁵-.

6. A compound as claimed in claim 1, wherein A and A³ independently represent —O—.

7. A compound as claimed in claim 1, wherein A⁴ represents a single bond, —C(O)— or —C(O)O—.

8. A compound as claimed in claim 7, wherein A⁴ represents —C(O)— or —C(O)O—.

9. A compound as claimed in claim 1, wherein A⁵ represents a single bond.

10. A compound as claimed in claim 1, wherein T represents C₁₋₃ alkylene, phenylene or a single bond.

11. A compound as claimed in claim 1, wherein Y represents —C(O)N(R^{10b})R^{9b}, —C(O)N(H)C(═NH)NH₂ or —C(O)N(H)S(O)₂R¹¹.

12. A compound as claimed in claim 1, wherein D represents a single bond or —O—.

13. A compound as claimed in claim 1, wherein R^{9a} to R^{9k} independently represent H or C₁₋₂ alkyl.

14. A compound as claimed in claim 1, wherein R^{10b}, R^{10d}, R^{10h}, R¹⁰ⁱ and R^{10k} independently represent heteroaryl optionally substituted by one or more C₁₋₃ alkyl groups, or, H or C₁₋₃ alkyl optionally substituted by one or more G¹ groups.

15. A compound as claimed in claim 14, wherein R^{10b}, R^{10d}, R^{10b}, R¹⁰ⁱ and R^{10k} independently represent H or C₁₋₃ alkyl optionally substituted by one or more G¹ groups.

16. A compound as claimed in claim 1, wherein R¹¹ represents C₁₋₂ alkyl.

17. A compound as claimed in claim 1, wherein X¹ represents —C(O)OR^{9a}, halo, Q-X² or H.

18. A compound as claimed in claim 17, wherein X¹ represents halo, Q-X² or H.

19. A compound as claimed in claim 1, wherein R¹, X² (when X² represents an aryl or heteroaryl group) and/or E represent optionally substituted phenyl, naphthyl, pyrrolyl, furanyl, thienyl, pyrazolyl, imidazolyl, oxazolyl, isoxazolyl, thiazolyl, pyridyl, indazolyl, indolyl, indolinyl, isoindolinyl, quinolinyl, 1,2,3,4-tetrahydroquinolinyl, isoquinolinyl, 1,2,3,4-tetrahydroisoquinolinyl, quinolizinyl, benzofuranyl, isobenzofuranyl, chromanyl, benzothienyl, pyridazinyl, pyrimidinyl, pyrazinyl, indazolyl, benzimidazolyl, quinazolinyl, quinoxalinyl, 1,3-benzodioxolyl, tetrazolyl, benzothiazolyl, and/or benzodioxanyl, groups.

20. A compound as claimed in claim 19, wherein R¹ and E independently represent optionally substituted pyridyl, phenyl or imidazolyl.

21. A compound as claimed in claim 19 or claim 20, wherein the optional substituents are selected from halo, cyano, —NO₂, C₁₋₆ alkyl (which alkyl group may be linear or branched, cyclic, part-cyclic, unsaturated and/or optionally substituted with one or more halo group), heterocycloalkyl (which heterocycloalkyl group is optionally substituted by one or more substituents selected from C₁₋₃ alkyl and ═O), —OR²¹ and —N(R²¹)R²², wherein R²¹ and R²² independently represent H or C₁₋₆ alkyl (which alkyl group is optionally substituted by one or more halo groups).

22. A compound as claimed in claim 1, wherein X² represents C₁₋₃ alkyl or heterocycloalkyl, both of which are optionally substituted by one or more G¹ and/or X³ groups.

23. A compound as claimed in claim 1, wherein R^{12a} to R^{12c} independently represent an imidazolyl, pyridyl, tetrazolyl group, pyrrolidinyl, piperidinyl, piperazinyl or morpholinyl group, or H or C₁₋₅ alkyl, which alkyl group is optionally substituted with one or more G³ groups.

24. A compound as claimed in claim 1, wherein R^{13a} to R^{13f} independently represent H or C₁₋₂ alkyl.

25. A compound as claimed in claim 1, wherein G³ represents halo or phenyl.

26. A compound as claimed in claim 1, wherein one of R⁴ and R³ represents -D-E and the other represents H.

27. A compound as claimed in claim 26, wherein R³ represents -D-E.

28. A compound as claimed in claim 1, wherein R² and/or R⁵ represent H.

29. A compound as defined in claim 1, but without the proviso, or a pharmaceutically-acceptable salt thereof, for use as a pharmaceutical.

30. A pharmaceutical formulation including a compound as defined in claim 1, but without the proviso, or a pharmaceutically-acceptable salt thereof, in admixture with a pharmaceutically acceptable adjuvant, diluent or carrier.

31. (canceled)

32. (canceled)

33. (canceled)

34. (canceled)

35. A method according to claim 36, wherein the disease is asthma, chronic obstructive pulmonary disease, pulmonary fibrosis, inflammatory bowel disease, irritable bowel syndrome, inflammatory pain, fever, migraine, headache, low back pain, fibromyalgia, a myofascial disorder, a viral infection, a bacterial infection, a fungal infection, dysmenorrhea, a burn, a surgical or dental procedure, a malignancy, hyperprostaglandin E syndrome, classic Bartter syndrome, atherosclerosis, gout, arthritis, osteoarthritis, juvenile arthritis, rheumatoid arthritis, rheumatic fever, ankylosing spondylitis, Hodgkin's disease, systemic lupus erythematosus, vasculitis, pancreatitis, nephritis, bursitis, conjunctivitis, iritis, scleritis, uveitis, wound healing, dermatitis, eczema, psoriasis, stroke, diabetes mellitus, a neurodegenerative disorder, an autoimmune disease, an allergic disorder, rhinitis, an ulcer, coronary heart disease, sarcoidosis, any other disease with an inflammatory component, osteoporosis, osteoarthritis, Paget's disease or a periodontal disease.

36. A method of treatment of a disease in which inhibition of the activity of a member of the MAPEG family is desired and/or required, which method comprises administration of a therapeutically effective amount of a compound as defined in claim 1, but without the proviso, or a pharmaceutically-acceptable salt thereof, to a patient suffering from, or susceptible to, such a condition.

37. A method as claimed in claim 36, wherein the member of the MAPEG family is microsomal prostaglandin E synthase-1, leukotriene C₄ and/or 5-lipoxygenase-activating protein.

38. A method as claimed in claim 37, wherein the member of the MAPEG family is microsomal prostaglandin E synthase-1.

39. A combination-product comprising:
(A) a compound as defined in any one of claim 1, but without the proviso, or a pharmaceutically-acceptable salt thereof; and
(B) another therapeutic agent that is useful in the treatment of inflammation,
wherein each of components (A) and (B) is formulated in admixture with a pharmaceutically-acceptable adjuvant, diluent or carrier.

40. A combination product as claimed in claim 39 which comprises a pharmaceutical formulation including a compound as defined in any one of claims 1 to 28, but without the proviso, or a pharmaceutically-acceptable salt thereof, another therapeutic agent that is useful in the treatment of inflammation, and a pharmaceutically-acceptable adjuvant, diluent or carrier.

41. A combination product as claimed in claim 39 which comprises a kit of parts comprising components:
(a) a pharmaceutical formulation including a compound as defined in any one of claims 1 to 28, but without the proviso, or a pharmaceutically-acceptable salt thereof, in admixture with a pharmaceutically-acceptable adjuvant, diluent or carrier; and
(b) a pharmaceutical formulation including another therapeutic agent that is useful in the treatment of inflammation in admixture with a pharmaceutically-acceptable adjuvant, diluent or carrier,
which components (a) and (b) are each provided in a form that is suitable for administration in conjunction with the other.

42. A process for the preparation of a compound as defined in claim 1, which comprises:
(i) reaction of a compound of formula II, 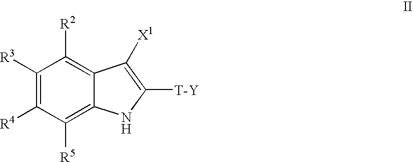
wherein X¹, R², R³, R⁴, R⁵, T and Y are as defined in claim 1, with a compound of formula III,
R¹L¹ III
wherein L¹ represents a suitable leaving group and R¹ is as defined in claim 1;
(ii) for compounds of formula I in which X¹ represents —C(O)OR^{9a}, —C(O)N(R^{10b})R^{9b}, —S(O)₂N(R^{10b})R^{9b}, —C(O)N(H)C(═NR^{9c})N(R^{10d})R^{9d}, —C(O)N(F)CN, —S(O)₃R^{9c}, —P(O)(OR^{9f})₂, —P(O)(OR^{9g})N(R^{10h})R^{9h}, —P(O)(N(R¹⁰ⁱ)R⁹ⁱ)₂, —B(OR^{9j})₂, —C(O)N(H)S(O)₂R¹¹ or -Q-X², in which Q is a single bond, —C(O)—, C₁₋₈ alkylene or C₂₋₈ heteroalkylene, reaction of a compound of formula IV, 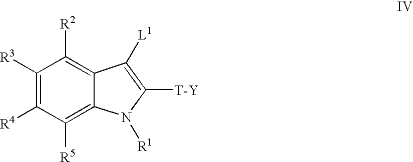
wherein R¹, R², R³, R⁴, R⁵, T and Y are as defined in claim 1 and L¹ is as defined above, with a compound of formula V,
X^{1a}-L² V
wherein X^{1a} represents —C(O)OR^{9a}, —C(O)N(R^{10b})R^{9b}, —S(O)₂N(R^{10b})R^{9b}, —C(O)N(H)C(═NR^{9c})N(R^{10d})R^{9d}, —C(O)N(H)CN, —S(O)₃R^{9e}, —P(O)(OR^{9f})₂, —P(O)(OR^{9g})N(R^{10h})R^{9h}, —P(O)(N(R¹⁰ⁱ)R⁹ⁱ)₂, —B(OR^{9j})₂, —C(O)N(H)S(O)₂R¹¹ or -Q-X², in which latter case Q is a single bond, —C(O)—, C₁₋₈ alkylene or
C₂₋₈ heteroalkylene, L² represents a suitable leaving group and R^{9a} to R^{9k}, R^{10b}, R^{10d}, R^{10h}, R¹⁰ⁱ, R^{10k} are as defined in claim 1;
(iia) for compounds of formula I in which X¹ represents —C(O)N(H)C(═NR^{9c})N(R^{10d})R^{9d}, —C(O)N(H)CN or —C(O)N(H)S(O)₂R′, reaction of either a compound of formula I, as defined in claim 1, in which X¹ represents H, or a compound of formula IV, as defined above, in which the L¹ group is activated, with a compound of formula VA,
R^{za}—N═C═O VA
wherein R^{za} represents —C(═NR^{9c})N(R^{10d})R^{9d}, —CN or —S(O)₂R¹¹, followed by quenching with a suitable proton source;
(iii) for compounds of formula I in which X¹ represents Q-X² and Q represents —C(O)—, reaction of a compound of formula I in which X¹ represents H with a compound of formula V in which Xia represents -Q-X², Q represents —C(O)— and L² represents a suitable leaving group;
(iv) for compounds of formula I in which X¹ represents —N(R^{9k})-J-R^{10k} or -Q-X² in which Q represents —O—, —S—, C₂₋₈ alkynylene or C₂₋₄ heteroalkylene in which latter two groups, the triple bond is adjacent to the indole ring of formula I, reaction of a compound of formula IV as defined above with a compound of formula VI,
X^{1b}H VI
in which X^{1b} represents —N(R^{9k})-J-R^{10k} or Q-X² in which Q represents —O—, —S—, C₂₋₈ alkynylene or C₂₋₈ heteroalkynylene, and R^{9k}, J, R^{10k} and X² are as defined in claim 1;
(v) for compounds of formula I in which X¹ represents -Q-X and Q represents —S—, reaction of a compound of formula I in which X¹ represents H, with a compound of formula VI in which X^{1b} represents -Q-X², Q represents —S— and X² is as defined in claim 1;
(vi) for compounds of formula I in which X¹ represents -Q-X² and Q represents —S(O)— or —S(O)₂—, oxidation of a corresponding compound of formula I in which Q represents —S—;
(vii) for compounds of formula I in which X¹ represents -Q-X², X² represents C₁₋₈ alkyl substituted by G¹, G¹ represents -A¹-R^{12a}, A¹ represents —N(R^{13a})A⁴- and A⁴ is a single bond (provided that Q represents a single bond when X² represents substituted C₁ alkyl), reaction of a compound of formula VII, 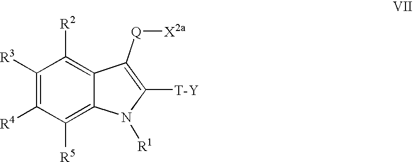
wherein X^{2a} represents a C₁₋₈ alkyl group substituted by a -Z¹ group in which Z¹ represents ═O, Q is as defined in claim 1, provided that it represents a single bond when X^{2a} represents C₁ alkyl substituted by ═O, and R¹, R², R³, R⁴, R⁵, T and Y are as defined in claim 1, under reductive amination conditions in the presence of a compound of formula VIII,
R^{12a}(R^{13a})NH VIII
wherein R^{12a} and R^{13a} are as defined in claim 1;
(viia) for compounds of formula I in which X¹ represents -Q-X², Q represents a single bond, X² represents methyl substituted by G¹, G¹ represents -A¹-R^{12a}, A¹ represents —N(R^{13a})A⁴- and A⁴ is a single bond, reaction of a corresponding compound of formula I in which X¹ represents H, with a mixture of formaldehyde (or equivalent reagent) and a compound of formula VIII as defined above;
(viii) for compounds of formula I in which X¹ represents -Q-X², Q represents a single bond and X² represents optionally substituted C₂₋₈ alkenyl (in which a point of unsaturation is between the carbon atoms that are t and e to the indole ring), reaction of a corresponding compound of formula I in which X¹ represents halo with a compound of formula IXA,
H₂C═C(H)X² IXA
or, reaction of a compound of formula VII in which Q represents a single bond and X^{2a} represents —CHO with either a compound of formula IXB,
(EtO)₂P(O)CH₂X^{2b} IXB
or the like, or a compound of formula IXC,
(Ph)₃P═C(H)X^{2b} IXC
or the like, wherein, in each case, X^{2b} represents H, X³, G¹ or C₁₋₆ alkyl optionally substituted with one of more substituents selected from X³, G¹ and/or Z¹ and X³, G¹ and Z¹ are as defined in claim 1;
(ix) for compounds of formula I in which X¹ represents -Q-X² and X² represents optionally substituted, saturated C₂₋₈ alkyl, saturated cycloalkyl, saturated C₂₋₈ heterocycloalkyl, saturated heterocycloalkyl, C₂₋₈ alkenyl, cycloalkenyl, C₂₋₈ heterocycloalkenyl or heterocycloalkenyl, reduction of a corresponding compound of formula I in which X² represents optionally substituted C₂₋₈ alkenyl, cycloalkenyl, C₂₋₈ heterocycloalkenyl, heterocycloalkenyl, C₂₋₈ alkynyl, cycloalkynyl, C₂₋₈ heterocycloalkynyl or heterocycloalkynyl (as appropriate);
(x) for compounds of formula I in which D represents a single bond, —C(O)—, —C(R⁷)(R⁸)—, C₂₋₄ alkylene or —S(O)₂—, reaction of a compound of formula X, 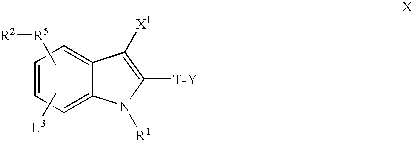
wherein L³ represents L¹ or L² as defined above, which group is attached to one or more of the carbon atoms of the benzenoid ring of the indole, R²—R⁵ represents whichever of the three other substituents on the benzenoid ring are already present in that ring, and X¹, R¹, R², R³, R⁴, R⁵, T and Y are as defined in claim 1, with a compound of formula XI,
E-D^{a}-L⁴ XI
wherein D^{a} represents a single bond, —C(O)—, —C(R⁷)(R⁸)—, C₂₋₄ alkylene or —S(O)₂—, L⁴ represents L¹ (when L³ is L²) or L² (when L³ is L¹), E, R⁷ and R⁸ are as defined in claim 1 and L¹ and L² are as defined above;
(xi) for compounds of formula I in which D represents —S—, —O— or C₂₋₄ alkynylene in which the triple bond is adjacent to E, reaction of a compound of formula X as defined above in which L³ represents L² as defined above with a compound of formula XII,
E-D^{b}-H XII
wherein D^{b} represents —S—, —O— or C₂₋₄ alkynylene in which the triple bond is adjacent to E and E is as defined in claim 1;
(xii) for compounds of formula I in which D represents —S(O)— or —S(O)₂—, oxidation of a corresponding compound of formula I in which D represents —S—;
(xiii) for compounds of formula I in which D represents —O— or —S—, reaction of a compound of formula XIII, 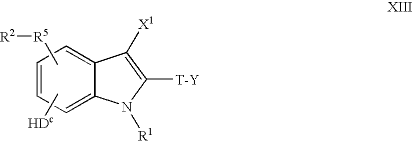
wherein the -DC-H group is attached to one or more of the carbon atoms of the benzenoid ring of the indole, D^{c} represents —O— or —S— and X¹, R¹, T and Y are as defined in claim 1, and R²—R⁵ is as defined above, with a compound of formula XIV,
E-L² XIV
wherein L² is as defined above and E is as defined in claim 1;
(xiv) for compounds of formula I in which X¹ represents —N(R^{9k})-J-R^{10k}, reaction of a compound of formula XV, 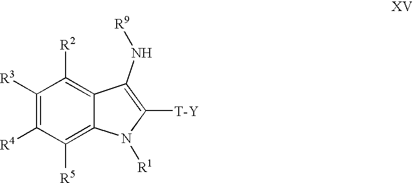
wherein R^{x}, R², R³, R⁴, R⁵, T, Y and R⁹ are as defined in claim 1, with a compound of formula XV¹,
R^{10k}-J-L¹ XVI
wherein J and R^{10k} are as defined in claim 1 and L¹ is as defined above;
(xv) for compounds of formula I in which X¹ represents —N(R^{9k})-J-R^{10k}, J represents a single bond and R^{10k} represents a C₁₋₈ alkyl group, reduction of a corresponding compound of formula I, in which J represents —C(O)— and R^{10k} represents H or a C₁₋₇ alkyl group, in the presence of a suitable reducing agent;
(xvi) for compounds of formula I in which X¹ represents halo, reaction of a compound of formula I wherein X¹ represents H, with a reagent or mixture of reagents known to be a source of halide atoms;
(xvii) for compounds of formula I in which T represents optionally substituted, saturated C₂₋₈ alkylene, saturated cycloalkylene, saturated C₂₋₈ heteroalkylene, saturated heterocycloalkylene, C₂₋₈ alkenylene, cycloalkenylene, C₂₋₈ heteroalkenylene or heterocycloalkenylene, reduction of a corresponding compound of formula I in which T represents optionally substituted C₂₋₈ alkenylene, cycloalkenylene, C₂₋₈ heteroalkenylene, heterocycloalkenylene, C₂₋₈ alkynylene, cycloalkynylene, C₂₋₈ heteroalkynylene or heterocycloalkynylene (as appropriate);
(xviii) for compounds of formula I in which X¹ represents -Q-X² and Q represents —O—, reaction of a compound of formula XVII, 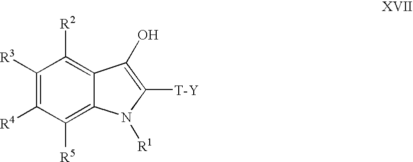
wherein R¹, R², R³, R⁴, R⁵, T and Y are as defined in claim 1, with a compound of formula XVIII,
X²L⁷ XVIII
wherein L⁷ represents a suitable leaving group and X² is as defined in claim 1;
(xix) reaction of a compound of formula XIX, 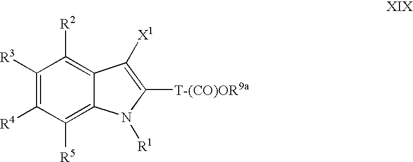
wherein R¹, R², R³, R⁴, R⁵, T, X¹ and R^{9a} are as defined in claim 1, with a compound of formula XX,
R²⁵(R²⁶)NH XX
wherein R²⁵ and R²⁶ represent, in the case of a compound of formula I in which Y represents:
(1) —C(O)N(R^{10b})R^{9b}, R^{9b} and R^{10b};
(2) —C(O)N(H)C(═NR^{9c})N(R^{10d})R^{9d}, —C(═NR^{9c})N(R^{10d})R^{9d} and H
(3) —C(O)N(H)CN, —CN and H; or
(4) —C(O)N(H)S(O)₂R¹¹, —S(O)₂R¹¹ and H,
respectively, and R^{9a} to R^{9d}, R^{10b}, R^{10d} and R¹¹ are as defined in claim 1;
(xx) for compounds of formula I in which X¹ is as defined in claim 1, provided that, when X¹ represents —C(O)OR^{9a}, —C(O)N(R^{10b})R^{9b}, —S(O)₂N(R^{10b})R^{9b}, —C(O)N(H)C(═NR^{9c})N(R^{10d})R^{9d}, —S(O)₃R^{9c}, —P(O)(OR^{9f})₂, —P(O)(OR^{9g})N(R^{10h})R^{9h}, —P(O)(N(R¹⁰ⁱ)R⁹ⁱ)₂, or —B(OR^{9j})₂, R^{9a} to R^{9g}, R⁹ⁱ, R^{9j}, R^{10b}, R^{10d} and R¹⁰ⁱ are other than H, reaction of a compound of formula XXI, 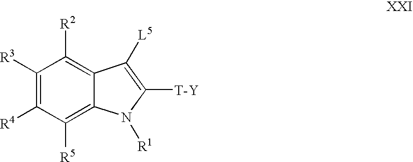
wherein L⁵ represents an appropriate alkali metal, a —Mg-halide, a zinc-based group or a suitable leaving group, and T, Y, R¹, R², R³, R⁴ and R⁵ are as defined in claim 1, with a compound of formula XXII,
L⁶-X^{1b} XXII
wherein X^{1b} represents X¹, provided that when X¹ represents —C(O)OR^{9a}, —C(O)N(R^{10b})R^{9b}, —S(O)₂N(R^{10b})R^{9b}, —C(O)N(H)C(═NR^{9c})N(R^{10d})R^{9d}, —S(O)₃R^{9e}, —P(O)(OR^{9f})₂, —P(O)(OR^{9g})N(R^{10h})R^{9h}, —P(O)(N(R¹⁰ⁱ)R⁹ⁱ)₂, or —B(OR^{9j})₂, R^{9a} to R^{9g}, R⁹ⁱ, R^{9j}, R^{10b}, R^{10d}, R^{10h} and R¹⁰ⁱ are other than H, or a protected derivative thereof, and L⁶ represents a suitable leaving group;
(xxi) for compounds of formula I in which X¹ represents —C(O)OR^{9a} and R^{9a} represents H, reaction of a compound of formula XXI in which L⁵ represents either:
(I) an alkali metal; or
(II) —Mg-halide,
with carbon dioxide, followed by acidification;
(xxii) for compounds of formula I in which X¹ represents —C(O)OR^{9a} or —C(O)N(R^{10b})R^{9b}, reaction of a corresponding compound of formula XXI in which Ls is a suitable leaving group with CO (or a reagent that is a suitable source of CO), in the presence of a compound corresponding to a compound of formula XXIIA,
R^{9a}OH XXIIA
wherein R^{9a} is as defined in claim 1, or a compound of formula XX as defined above in which R²⁵ and R²⁶ represent R^{9b} and R¹⁰ b respectively, and an appropriate catalyst system;
(xxiii) for compounds of formula I in which X¹ represents —B(OR^{9j})₂ and R^{9j} represents H, reaction of a compound of formula XXI as defined above with boronic acid or a protected derivative thereof and an appropriate catalyst system, followed by (if necessary) deprotection;
(xxiv) for compounds of formula I in which X¹ represents —S(O)₃R^{9e} or —S(O)₂N(R^{10b})R^{9b}, reaction of a compound of formula XXI as defined above with:
(A) for compounds of formula I in which X¹ represents —S(O)₃R^{9e}, and R^{9e} represents H, either SO₃ or with SO₂ followed by treatment with N-chlorosuccinimide and then hydrolysis;
(B) for compounds of formula I in which X¹ represents —S(O)₃R^{9e}, and R^{9e} is other than H, chlorosulfonic acid followed by reaction with a compound of formula XXIII as defined below in which R^{9za} represents R^{9e};
(C) for compounds of formula I in which X¹ represents —S(O)₂N(R^{10b})R^{9b}, chlorosulfonic acid followed by reaction with a compound of formula XX as defined above;
(xxv) for compounds of formula I in which Q represents optionally substituted C₂₋₈ alkenylene or C₂₋₈ heteroalkenylene (in which a point of unsaturation is between the carbon atoms that are É and é to the indole ring), reaction of a compound of formula VII in which Q represents a single bond and X^{2a} represents —CHO with a compound of formula XXIIB,
(Ph)₃P═C(H)-Q^{c}-X¹ XXIIB
or the like, wherein Q^{c} represents a single bond or optionally substituted C₁₋₆ alkylene or C₂₋₆ heteroalkylene and X¹ is as defined in claim 1;
(xxvi) for compounds of formula I in which Q represents optionally substituted, saturated C₂₋₈ alkylene, saturated cycloalkylene, saturated C₂₋₈ heteroalkylene, saturated heterocycloalkylene, C₂₋₈ alkenylene, cycloalkenylene, C₂₋₈ heteroalkenylene or heterocycloalkenylene, reduction of a corresponding compound of formula I in which Q represents optionally substituted C₂₋₈ alkenylene, cycloalkenylene, C₂₋₈ heteroalkenylene, heterocycloalkenylene, C₂₋₈ alkynylene, cycloalkynylene, C₂₋₈ heteroalkynylene or heterocycloalkynylene (as appropriate);
(xxvii) for compounds of formula I in which X¹ represents —C(O)OR^{9a}, —S(O)₃R^{9e}, —P(O)(OR^{9f})₂ or —B(OR^{9j})₂, in which R^{9a}, R^{9e}, R^{9f} and R^{9j} represent H, hydrolysis of a corresponding compound of formula I in which R^{9a}, R^{9e}, R^{9f} and R^{9j} do not represent H, or, for compounds of formula I in which X¹ represents —C(O)OR^{9a} or
—P(O)(OR^{9f})₂, in which R^{9a} and R^{9f} represent H, a corresponding compound of formula I in which X¹ represents —C(O)N(H)S(O)₂R¹¹, —P(O)(OR^{9g})N(R^{10h})R^{9h} or —P(O)(N(R₁₀ᵢ)R⁹ⁱ)₂ (as appropriate);
(xxviii) for compounds of formula I in which X¹ represents —C(O)OR^{9a}, —S(O)₃R^{9e}, —P(O)(OR^{9f})₂, —P(O)(OR^{9g})N(R^{10h})R^{9h} or —B(OR^{9j})₂ and R^{9a}, R^{9e}, R^{9f}, R^{9g} and R^{9j} do not represent H:
(A) esterification of a corresponding compound of formula I in which R^{9a}, R^{9c}, R^{9f}, R^{9g} and R^{9j} represent H; or
(B) trans-esterification of a corresponding compound of formula I in which R^{9a}, R^{9e}, R^{9f}, R^{9g} and R^{9j} do not represent H (and do not represent the same value of the corresponding R^{9a}, R^{9e}, R^{9f}, R^{9g} and R^{9j} group in the compound of formula I to be prepared),
in the presence of the appropriate alcohol of formula XXIII,
R^{9za}OH XXIII
in which R^{9za} represents R^{9a}, R^{9e}, R^{9f}, R^{9g} or R^{9j} provided that none of those R⁹ groups represent H;
(xxix) for compounds of formula I in which Q represents a C₁ alkylene group substituted with G¹, in which G¹ represents -A¹-R^{2a}, A¹ represents —C(O)A²-, A² represents a single bond and R^{12a} represents H, and X² represents —C(O)OR^{9a}, in which R^{9a} is other than H, reaction of a corresponding compound of formula I in which the C₁ alkylene group that Q represents is unsubstituted with C₁₋₆ alkyl formate in the presence of a suitable base;
(xxx) for compounds of formula I in which X¹ represents —C(O)N(R^{10b})R^{9b}, —C(O)N(H)C(═NR^{9c})N(R^{10d})R^{9d}, —C(O)N(H)CN or —C(O)N(H)S(O)₂R′ 1 reaction of a corresponding compound of formula I in which X¹ represents —C(O)OR^{9a} with a compound of formula XX as defined above;
(xxxi) for compounds of formula I in which X¹ represents -Q-X², Q represents a single bond and X² represents C₁₋₈ alkyl or heterocycloalkyl substituted α to the indole ring by a G¹ substituent in which G¹ represents -A¹-R^{12a}, A¹ represents
—OA⁵-, A⁵ represents a single bond and R^{12a} represents H, reaction of a corresponding compound of formula I in which X¹ represents H with a compound corresponding to a compound of formula VI, but in which X^{1b} represents -Q-X², Q represents a single bond and X² represents C₁₋₈ alkyl or heterocycloalkyl, both of which groups are substituted by a Z¹ group in which Z¹ represents ═O;
(xxxii) for compounds of formula I in which X¹ represents -Q-X², Q represents a single bond and X² represents C₂₋₈ alkyl substituted by a G¹ substituent in which G¹ represents -A¹-R^{12a}, A¹ represents —OA⁵-, A⁵ represents a single bond and R^{12a} represents H, reaction of a corresponding compound of formula I in which X² represents C₁₋₇ alkyl substituted by a Z¹ group in which Z¹ represents ═O, with the corresponding Grignard reagent derivative of a compound of formula V in which L² represents chloro, bromo or iodo, X^{1a} represents -Q-X², Q is a single bond and X² represents C₁₋₇ alkyl;
(xxxiii) for compounds of formula I in which X¹ represents -Q-X², Q represents a single bond, and X² represents C₁₋₈ alkyl or heterocycloalkyl, both of which are unsubstituted in the position (to the indole ring, reduction of a corresponding compound of formula I in which X² represents C₁₋₈ alkyl substituted α to the indole ring by a G¹ substituent in which G¹ represents -A¹-R^{12a}, A¹ represents
-OA⁵-, A⁵ represents a single bond and R^{12a} represents H;
(xxxiv) for compounds of formula I in which X¹ represents -Q-X², Q represents a single bond and X² represents C₁₋₈ alkyl or heterocycloalkyl, neither of which are substituted by Z¹ in which Z¹ represents ═O, reduction of a corresponding compound of formula I in which X² represents C₁₋₈ alkyl or heterocycloalkyl, which groups are substituted by one or more Z¹ groups in which Z¹ represents ═O; or
(xxxv) for compounds of formula I in which X¹ represents —N(R^{9k})-J-R^{10k}, reaction of a compound of formula XVII as defined above, with a compound of formula VI in which X^{1b} represents —N(R^{9k})-J-R^{10k} and R^{9k}, R^{10k} and J are as defined in claim 1.
